(19) United States
(12) Patent Application Publication
(10) Pub. No.: US 2026/0193303 A1
(43) Pub. Date: Jul. 9, 2026

(54) SYNTHETIC MANIPULATION OF MAMMALIAN SECRETORY PHENOTYPES FOR AUGMENTED BIOPRODUCTION

(71) Applicant: William Marsh Rice University, Houston, TX (US)

(72) Inventors: Caleb J. BASHOR, Houston, TX (US); John HER, Houston, TX (US); Xiaoyu YANG, Houston, TX (US); Zheng DIAO, Houston, TX (US); Andrew J. WALTERS, Houston, TX (US)

(21) Appl. No.: 19/376,386

(22) Filed: Oct. 31, 2025

Related U.S. Application Data
(60) Provisional application No. 63/714,521, filed on Oct. 31, 2024.

(51) Int. Cl. C07K 14/47; C07K 14/575; C07K 16/00; C12N 15/86

(52) U.S. Cl. CPC C07K 14/47; C07K 14/575; C07K 16/00; C12N 15/86; C12N 2740/15043; C12N 2740/15051

(57) ABSTRACT

There is a growing need for enhancements to human workhorse cell lines (e.g., HEK293 cells) critical for synthesis of specific therapeutic products such as viral vectors for gene therapies. To that end, aspects of the disclosure are drawn to genetically engineered cells and Methods of boosting bioproduction capacity in mammalian cells.

This application is an United States non-provisional application which claims priority from U.S. patent application No. 63/714,521 filed on Oct. 31, 2024, the entire contents of each which are incorporated herein by reference.

### GOVERNMENT INTERESTS

This invention was made with government support under Grant No. N00014-21-1-4006 awarded by the Office of Naval Research and Grant No. EB029483 awarded by the National Institutes of Health. The government has certain rights in the invention.

For countries that permit incorporation by reference, all patents, patent applications and publications cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Documents incorporated by reference into this text are not admitted being prior art.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### REFERENCE TO A SEQUENCE LISTING XML

This application contains a Sequence Listing which has been submitted electronically in XML format. The Sequence Listing XML is incorporated herein by reference. Said XML file, created on Feb. 4, 2026, is named 2967398-000040-US2_SL.xml and is 94,883 bytes in size.

### BACKGROUND

In recent decades, mammalian cell lines have become vital to the production of an ever-growing list of advanced biologic medicines, including immunoglobulin-based therapeutics, soluble immunoregulatory ligands, and catalytic biologics. To date, strategies for improving production have primarily focused on maximizing cell health, culture density, and productivity by optimizing media formulations and bioreactor control strategies. Efforts to improve the secretory output are constrained by the specific secreted product and cell line contexts, with limited generalizability. There is a growing need for enhancements to human workhorse cell lines (e.g., HEK293 cells) critical for synthesis of specific therapeutic products such as viral vectors for gene therapies.

### SUMMARY OF THE INVENTION

An aspect of the disclosure is drawn towards a genetically engineered cell comprising an expression vector, wherein the expression vector encodes for two or more recombinant proteins selected from the group consisting of STXBP1, SNAP23, SNAP25, SNAP91, VAMP2, VAMP7, BNIP1, GOSR1, BET1, SEX22B, YKT6, SCFD1, SAR1B, SEC24A, and STXBP1. For example, the two or more recombinant protects comprises STXBP1, SEC24A, and SNAP23.

In some embodiments, the two or more recombinant proteins are expressed from one or more plasmids or from a multi-gene plasmid.

In some embodiments, the cell comprises a mammalian cell. For example, the mammalian cell comprises a HEK293T cell, a retinal pigment epithelial cell, or a mesenchymal stem cell.

In some embodiments, the cell comprises an encapsulated cell.

In some embodiments, the cell is engineered to express and/or secrete a biomolecule of interest. For example, the biomolecule of interest comprises a recombinant protein or polypeptide or a nucleic acid. For example, the recombinant protein or polypeptide comprises a prohormone, a hormone, a blood clotting protein, an antibody or antigen binding fragment thereof, a protein receptor, a fusion protein, a vaccine, a cytokine, a chemokine, a growth factor, or a blood factor protein. For example, the nucleic acid comprises a recombinant virus or viral vector.

Further, aspects of the disclosure are drawn towards a vector encoding two or more recombinant proteins selected from the group consisting of STXBP1, SNAP23, SNAP25, SNAP91, VAMP2, VAMP7, BNIP1, GOSR1, BET1, SEX22B, YKT6, SCFD1, SAR1B, SEC24A, and STXBP1. For example, the two or more recombinant protects comprises STXBP1, SEC24A, and SNAP23.

In some embodiments, the vector comprises one or more additional regulatory elements.

Further, the disclosure is drawn towards a cell comprising the vector. For example, the cell comprises a mammalian cell. For example, the mammalian cell comprises a HEK293T cell, a retinal pigment epithelial cell, or a mesenchymal stem cell.

In some embodiments, the cell comprises an encapsulated cell.

Aspects of the disclosure are further drawn towards a method for producing a biomolecule of interest.

In some embodiments, the method comprises culturing a population of mammalian cells, wherein the population of mammalian cells are genetically engineered to express two or more recombinant proteins selected from the group consisting of STXBP1, SNAP23, SNAP25, SNAP91, VAMP2, VAMP7, BNIP1, GOSR1, BET1, SEX22B, YKT6, SCFD1, SAR1B, SEC24A, and STXBP1, wherein the population of mammalian cells are further genetically engineered to express the biomolecule; and recovering said biomolecule of interest from the medium from said culture.

In some embodiments, the two or more recombinant proteins synergize to increase production and/or secretion of the biomolecule.

Some embodiments further comprise transducing the population of mammalian cells with one or more vectors expressing the two or more recombinant proteins and/or the biomolecule.

Still further, aspects of the disclosure are drawn towards a genetically engineered cell comprising a synthetic secretory pathway.

In some embodiments, the synthetic secretory pathway comprises a recombinant membrane receptor configured to sense a signaling compound, a recombinant calcium ion channel, and a recombinant secretion component, wherein the membrane receptor and the recombinant secretion component are operably linked by the recombinant calcium ion channel.

In some embodiments, the cell comprises a mammalian cell. For example, the mammalian cell comprises a HEK293T cell, a retinal pigment epithelial cell, or a mesenchymal stem cell.

In some embodiments, the cell comprises an encapsulated cell.

In some embodiments, the cell comprises an expression vector encoding for two or more recombinant proteins selected from the group consisting of SYT7, STX4, RAB27A, and RAB27B.

An aspect of the disclosure is directed to constitutive secretion boosters, for example, STXBP1, SEC24A, and SNAP23, that can improve yields from common ex vivo biomanufacturing platforms for products such as recombinant protein and virus.

Aspects of the disclosure are also drawn towards regulated secretion, such as “sense-and-secrete” programs, that allow for the development of implanted cell factories that pre-manufacture a biomolecule (e.g., a therapeutic such as insulin), then release it all at once on demand or when a “trigger” is sensed.

Still further, aspects of the disclosure are drawn towards a cell line optimized for secretion of a biomolecule, improving downstream processing and cost of goods

Further, aspects of the disclosure are drawn towards an encapsulated cell product programmed with “sense-and-secrete” that can be used as an implantable cell factory with rapid secretion capabilities, vs. slow constitutive secretion from other platforms.

Other objects and advantages of this invention will become readily apparent from the ensuing description.

### BRIEF DESCRIPTION OF THE FIGURES

FIG. 1A shows bioinformatic analysis of constitutive secretion factors, normalized to expression levels in professional secretory cell types. HEK cells are markedly deficient in expression of these factors compared to specialists. FIG. 1B shows binary secretory factor screens show pairs of factors produce certain synergistic behaviors. FIG. 1C shows screened higher order (tertiary) combinations with respect to an identified factor STXBP1 to formulate the constitutive module.

FIG. 2A shows quantification of intracellular and secreted products in our constitutive secretion module. Identified tertiary combination has the best secretory output, with cargos leaving the intracellular pool. FIG. 2B shows quantification of intracellular compartment localization via microscopy.

FIG. 3A shows demonstration of monoclonal antibody secretion with identified constitutive secretion module. mAb production increases with the addition of the module. FIG. 3B shows demonstration of virus production using constitutive secretion module, showing marked improvement as well. FIG. 3C shows IL-10 secretion in MSCs demonstration shows the module increases passive secretion in a primary therapeutic cell type. FIG. 3D shows demonstration of protein secretion in ARPE-19 cells, clinically compatible cell type for encapsulation.

FIG. 4A shows bioinformatic analysis of regulated secretion factors, categorized by related intracellular functions. HEKs are expression deficient. FIG. 4B shows binary screening of regulated factors, by measuring secretion when ionophore activated. FIG. 4C shows higher order regulated SF combinations, we identified a 4-protein module that had optimal secretory induction upon ionophore addition.

FIG. 5A shows quantification of engineered secretory reporter's intracellular localization. FIG. 5B shows imaging of granule formation and release over time. FIG. 5C shows time course of secretion of engineered cargos.

FIG. 6A and FIG. 6B shows construction of calcium activation channel pathway and reporter of calcium influx. FIG. 6C shows connecting the small molecule binding receptor to calcium channel activation and the secretion modules to build full sense and secrete functionality.

FIG. 7 shows bioinformatic comparison of secretory protein expression in professional secretory cell types and HEK-293T cells. Relative expression levels of HEK293T cells for genes we screened to develop constitutive secretory programs, in comparison to an STXBP-enriched professional secretory baseline. Relative expression levels of several cell and tissue types compared to the professional secretory baseline. A ratio below 0.0001 is flagged as “low.” Index of STXBP enrichment among cell types from a Protein Atlas single-cell type RNA-seq dataset. These data were used to construct high-STXBP and low-STXBP secretory archetypes used for bioinformatic analyses.

FIG. 8 shows diagram of hierarchical golden gate cloning cloning Workflow. A multi-level modular hierarchical cloning pipeline, facilitated by use of Type IIS restriction enzymes, allows us to efficiently construct complex multigene cellular programs. The alternation between antibiotic resistances encoded by each plasmid construct layer, coupled with ccdB selection against undigested vector, ensures high assembly efficiency. SP-cargo-mCh2 pictured here denotes an example of this strategy being employed to construct a fluorescent secretory reporter, which in this case is paired with a chromatin insulator, promoter, Kozak sequence (K100 for default-efficiency canonical sequence), and transcriptional terminator.

FIG. 9 shows payload generalizability of secretory reporter design. mCherry2 is an oxidation-resistant fluorophore protein, making it suitable for measuring secretory output and function via fluorescence as secretory organelle machinery are oxidative compartments; the secrecon tag functions as a signal peptide so that the cell will present and release the protein at the membrane-interface. Various protein payloads of interest such as erythropoietin (EPO) and Factor IX can be tagged with these two components. This design was generalizable to payloads secreted via regulated secretion pathways (those requiring CHGA-mediated granule formation) as well. FIG. 9 discloses SEQ ID NOS 53, 84, 54, 55, 84, 85, 22, 54, 55, 84, 86, 22, 54, 55, 87, 22, 54, 55, 59, 60, 88-92, 54 and 55, respectively, in order of appearance.

FIG. 10 shows retention and secretion validation of secretory payloads tagged with mCherry2. (a) Bar plots show secretory output w.r.t. mCherry fluorescence in intracellular and external compartments, when transfecting reporter construct at different concentrations (with remaining transfection mass being pcDNA). (b) Bar plots show the effects co-transfecting reporter construct (sec-mCh2 payload) with secretion factor construct (100 ng of reporter and 900 ng of secretion factor construct transfected, driven by D1-CMV promoter). Red dotted line indicates reference normalization standard.

FIG. 11A shows time course activity of secretory cargo and cells. FIG. 11A shows intracellular samples were acquired via cell lysis and secreted samples were acquired via extraction of cell secretion media samples for plate reader analysis at various timepoints. FIG. 11B shows secretory proteins traffic through intracellular compartments before being deposited extracellularly via the plasma membrane. Nascent proteins with secretion tags are transported from the endoplasmic reticulum to the golgi apparatus and subsequently the plasma membrane for exocytosis, and transfer of cargo at each of these steps is guided by sets of key secretion factors. FIG. 11C shows secretion output and intracellular output of secretory payload were measured over a 48-hour period. FIG. 11D shows cells were counted in tandem with 48-hour secretory assay to quantify cell health over 48-hour period when transfected with the reporter.

FIG. 12A shows binary synergy screening of secretory factors. FIG. 12A shows heatmap of interaction coefficients calculated using the methodology described in Klumpe et al. Interaction coefficients summarize the excess secretory enhancement brought on by pairs of constructs versus predicted secretory activity from single parts. FIG. 12B shows scatter plot shows correlation between interaction coefficient and measured secretion output (mCherry fluorescence), with R²=0.87.

FIG. 13 shows screening of secretory signal peptides. HEK293T cells were transfected with 100 ng of reporter construct fused to variable signal peptide, 450 ng of STXBP1 driven by D1-CMV, 450 ng of SEC24A driven by D1-CMV. Signal peptides were selected from common secretory products of interest in pharmaceuticals or experimental demonstration. The secrecon signal peptide (HMM+38) for further screening work due to its sufficiently low reporter-only fluorescence. Red dotted line indicates reference normalization standard. FIG. 13 discloses SEQ ID NOS 14, 13, 12, 15, 16, 5, 17, 18, 20, 11, 19, 9, 8, 7, 6, 10, 4, 3, 21, 2 and 1, respectively, in order of appearance.

FIG. 14 shows stoichiometry effects of secretory factor transfection in single expression unit compared to multiexpression unit constructs. Stoichiometrically balanced single-plasmid multigene programs were transfected using either PEI or jetPRIME into HEK293T cells to boost secretion of fluorescent reporters. PEI-based transfections were 1000 ng/well (24-well format), whereas jetPRIME transfections were 500 ng/well. 100 ng of reporter (sec-mCh2 or SP_IL6-mCh2) was co-transfected with a 116-fmol program using PEI. 116 fmol is equivalent to 900 ng of the largest program evaluated. Subplots describe secreted versus intracellular contents of sec-mCh2 in HEK293T cells transfected with either PEI or jetPRIME (all plasmid quantities halved for jetPRIME). Red dotted line indicates reference normalization standard.

FIG. 15 shows distribution workflow for super-resolution microscopy and image analysis. Top left: HEK293T cells are transfected with a combination of constructs to label subcellular compartments, including membrane-tagged iRFP, sec-mCherry (sec-mCH), and multi-secretion factors expressed on multiexpression unit cassettes. Cells are replated in multi-well chamber slides and imaged using structured illumination microscopy (SIM). Middle left: Snapshot images of individual cells show fluorescence signals for the ER (blue), Golgi (green), membrane (magenta), and reporter (red). Bottom left: Custom segmentation algorithms are applied to generate masks for the ER, Golgi, and membrane compartments, enabling quantitative analysis of fluorescence distribution within each cellular region. Right: Intensity distributions for the ER, Golgi, and membrane compartments are plotted as histograms (gray bars). Segmentation thresholds (dashed lines) are applied to distinguish specific compartmental fluorescence from background signals. Pixel intensities above these thresholds are summed to quantify compartment-specific fluorescence.

FIG. 16 shows overview of MSC flow cytometry workflow. MSC flow cytometry and gating workflow. A representation of a typical transient experiment. A known number of input cells are nucleofected with a reporter construct, then measured at 48 hours. The cells are counted and the population is phenotyped using flow cytometry. Events were gated by forward (FSC-A) and side scatter area (SSC-A) to yield a population of cells. Single cells were gated by forward scatter height (FSC-H) vs area (FSC-A). The percentage of BFP+ single cells was then recorded as the nucleofection efficiency. Overall yield was calculated from the counted number of cells harvested divided by the known input number of cells. The BFP+ yield was then calculated by multiplying the overall yield by the nucleofection efficiency.

FIG. 17 shows comparison of transfection method Lipofectamine 3000 and Jetprime transfection in ARPE-19 cells. ARPE-19 cells were cultured and transfected using methods described in Example 1 with 50 ng of reporter construct, 450 ng of secretion factor multiexpression unit construct. Red dotted line indicates reference normalization standard.

FIG. 18 shows experimental demonstration of constitutive secretion of High-Value Pharmaceutical Payloads. HEK293T cells were transfected with PEI method, 100 ng of reporter construct (mCherry2 fused with variable proteins of interest), and 900 ng of multiexpression unit construct consisting of STXBP1 and SEC24A. Red dotted line indicates reference normalization standard.

FIG. 19 shows standard curve of transturamab antibody titering assay. Transturamab is a monoclonal antibody that targets and binds to the human epidermal growth factor receptor 2 (HER2) protein on cancer cells and blocks HER2 signaling. As shown herein, anti-HER2 yield was quantified using the Easy-Titer assay system, an absorbance plate-reader method using antibody-sensitized microspheres that agglutinate in the presence of their target, in this case the human IgG H+L structure. Standard curve was fitted using mAb standard measurement; experimental samples were diluted to be in dynamic range of standard curve.

FIG. 20 shows bioinformatic analysis of genes involved in regulated secretion. Comparison of identified regulated secretion proteins between professional secretory cell types and cell types of interest in this paper. Relative expression levels of HEK293T cells for genes we screened to develop regulated secretory circuits, in comparison to a SYT-enriched professional secretory baseline. Relative expression levels of several cell and tissue types compared to the professional secretory baseline. Index of SYT enrichment among cell types from a Protein Atlas single-cell type RNA-seq dataset. These data were used to construct high-SYT and low-SYT secretory archetypes used for bioinformatic analyses.

FIG. 21A shows screening of secretion factors in regulated secretion context. Secretion factor proteins involved in packaging, vesicle formation and delivery were identified as regulated factors that could be boosted. FIG. 21A shows heatmap shows measured secretion output from co-transfecting SYT family secretion factor proteins with known packaging proteins, using the mCh2 based reporter fused with ChgA, a granule forming protein factor. FIG. 21B shows heatmap shows measured secretion output from co-transfecting regulated secretion factors with constant constitutive secretion factors (SYT7 and STX4), for quantenary screening.

FIG. 22A and FIG. 22B shows comparison of PEI transfection of single expression unit regulated secretion factors against jetPrime transfection of multi-expression unit secretion Factors. Selected regulated secretion factors were screened and shown in comparison here using PEI transfection method with individual expression units and jetPrime transfection for multi-gene arrays. We identified the RAB26, RAB27, SYT7, and STX4 combinations to exhibit ideal on/off behavior when induced with ionophore. Red dotted line indicates reference normalization standard.

FIG. 23 shows screening of intrinsically disordered protein linker regions with reporter. We fused intrinsically disordered protein sequences as a substitute flexible linker between ChgA and mCh2 domains in the reporter. These 5 IDRs were variations on repeating RGG motifs. We identified the variant containing IDR1 to move forward with testing for its favorable ON/OFF secretion output (fold-change). Red dotted line indicates reference normalization standard.

FIG. 24 shows optimizing the regulated secretory circuit payload. HEK293T cells were transfected with our optimized 4-SF program (using PEI method) in various configurations to decide on a regulated secretory circuit layout for subsequent development of sense-and-secrete circuits. The same 4-SF circuit yields different performance characteristics depending on the use of single expression unit or multiexpression unit cassettes, as well as the order of expression units on multiexpresion unit cassettes. In all conditions, 1000 ng was transfected: 100 ng of the reporter, along with equal masses of each secretion factor was used (225 ng per single expression unit, 450 ng per multiexpression unit cassette). We identified our multiexpression unit construct with RAB27B, RAB26 co-transfected with single expression unit constructs of SYT7 and STX4 to be optimal for regulated secretion for further sense-and-secrete engineering. Red dotted line indicates reference normalization standard.

FIG. 25 shows inducible secretion workflow and granule segmentation. Top left: HEK293T cells are transfected with constructs to express fluorescently labeled vesicles and plated in chamber slides for imaging. Middle left: Snapshot z-stack images are acquired using structured illumination microscopy (SIM) to capture the spatial distribution of secretory granules. Top right: Granules are classified into small and large categories based on fluorescence intensity and size. The fluorescence intensity distribution for small (left) and large (right) granules is plotted as histograms (shaded red), showing distinct intensity profiles. Bottom left: Granule segmentation is performed using custom MATLAB algorithms. The initial segmented mask identifies all detected granules. A subsequent watershed algorithm is applied to separate closely spaced granules, resulting in a refined segmented mask. Bottom right: The fluorescence intensity distribution of all detected granules is plotted (gray histogram), with a segmentation threshold (dashed line) applied to distinguish individual granules from background noise. Granule features such as volume, mean intensity, and centroid coordinates are extracted, and total fluorescence intensity is quantified for each vesicle. The distribution of granule intensities is further analyzed to assess changes before and after ionophore induction over a 3-hour period.

FIG. 26 shows flow cytometry workflow for HEK transduction and transfection. Representations of flow cytometry workflow experiment. Cells were measured on a 48 hr timescale in both cases. Transfection workflow requires small molecule or ionophore induction prior to final flow cytometry measurement via Ca2+ reporter gene. Cells were gated on BFP in transfection workflow, and eGFP on transduction workflow.

FIG. 27 shows development of a Ca2+-induced fluorescent transcriptional reporter. Co-transfection of NFAT4 into HEK293T allows for use of synthetic pNFAT promoters, which upregulate intracellular sfGFP reporter expression in response to Ca2+ influx from the plasma membrane. sfGFP fluorescence can be measured as a proxy for cytosolic calcium influx mediated by ORAI channel activity. Gating noted on Supplementary F20.

FIG. 28 shows canonical interactions between ORAI1 Ca2+ Channel and SOAR domain of STIM1. Full-length STIM responds to endoplasmic reticulum (ER) intraluminal Ca2+ depletion by exposing its SOAR domain. SOAR binds and opens the Orai plasma membrane-localized Ca2+ channel. The Orai family of plasma membrane Ca2+ channels, ubiquitous across cell types and tissues, is primarily responsible for store-operated Ca2+ entry (SOCE) in response to emptied ER Ca2+ stores. The ER-bound transmembrane protein STIM1 senses emptied ER luminal Ca2+ content and responds by conformational changes that expose its STIM-Orai activating region (SOAR), which binds to and opens Orai channels to allow for restoration of ER Ca2+ reserve.

FIG. 29 shows two modes of secretion—constitutive secretion versus regulated secretion. Regulated secretion is superimposed onto post-Golgi constitutive secretion, but only specialized cells (e.g., neuroendocrine cells) perform it. The regulated secretory pathway accumulates a reserve of secretory cargo until induced to secrete

FIG. 30 shows selecting an optimized regulated secretory circuit design.

### DETAILED DESCRIPTION OF THE INVENTION

### A. Abbreviations and Definitions

Detailed descriptions of one or more embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

“Consisting essentially of” when used to define compositions and methods, can refer to the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like.

“Consisting of” shall mean excluding more than trace elements of other ingredients and substantial method steps for administering the compositions provided and/or claimed in this disclosure. Embodiments defined by each of these transition terms are within the scope of this disclosure.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal 10 to 10” is also disclosed. It is also understood that throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Optional” or “optionally” means that the subsequently described event or circumstance may or may not occur, and that the description includes instances where said event or circumstance occurs and instances where it does not.

An “increase” can refer to any change that results in a greater amount of a composition (e.g., biomolecule of interest), condition or activity (e.g., gene expression). An increase can be any individual, median, or average increase in a composition, condition, or activity, in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a lesser amount of a composition (e.g., biomolecule of interest), condition or activity (e.g., gene expression). A decrease can be any individual, median, or average increase in a composition, condition, or activity, in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease statistically significant.

A “control” can refer to an alternative subject or sample used in an experiment for comparison purposes. A control can be “positive” or “negative.”

“Encoding” can refer to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom, Thus, a gene encodes a protein if transcription and translation of mRNA occurs.

A “protein”, “polypeptide”, or “peptide” each can refer to a polymer of amino acids and does not imply a specific length of a polymer of amino acids. Thus, for example, the terms peptide, oligopeptide, protein, antibody, and enzyme are included within the definition of polypeptide. This term also includes polypeptides with post-expression modification, such as glycosylation (e.g., the addition of a saccharide), acetylation, phosphorylation, and the like.

The term “nucleic acid” can refer to a polymer composed of nucleotides, e.g., deoxyribonucleotides or ribonucleotides.

The term “nucleobase” can refer to the part of a nucleotide that bears the Watson/Crick basepairing functionality. The most common naturally-occurring nucleobases, adenine (A), guanine (G), uracil (U), cytosine (C), and thymine (T) bear the hydrogen-bonding functionality that binds one nucleic acid strand to another in a sequence specific manner.

The terms “ribonucleic acid” and “RNA” can refer to a polymer composed of ribonucleotides.

The terms “deoxyribonucleic acid” and “DNA” can refer to a polymer composed of deoxyribonucleotides.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Genetically Engineered Cells and Methods of Boosting Bioproduction Capacity in Mammalian Cells

Mammalian cell lines have become vital to the production of an ever-growing list of advanced biologic medicines, including immunoglobulin-based therapeutics, soluble immunoregulatory ligands, and catalytic biologics. Strategies for improving production have primarily focused on maximizing cell health, culture density, and productivity by optimizing media formulations and bioreactor control strategies. Further, efforts to improve the secretory output are constrained by the specific secreted product and cell line contexts, with limited generalizability.

To that end, the present inventors have surprisingly discovered that they can synergistically boost bioproduction capacity in mammalian cells through the ectopic expression of secretory pathway factors. Nascent proteins with secretion tags are transported from the endoplasmic reticulum to the golgi apparatus and subsequently the plasma membrane for exocytosis, and transfer of cargo at each of these steps is guided by sets of key secretion factors. Secretion factor proteins involved in packaging, vesicle formation and delivery were identified as regulated factors that could be harnessed to boost biomolecule production in mammalian cells. Further, this synergism is generalizable to diverse cell contexts and biomanufacturing applications.

Accordingly, in one aspect, disclosed herein are methods for producing a biomolecule of interest (such as, for example, a recombinant protein or polypeptide (including, but not limited to enzymes, prohormones (e.g., Chromogranin A), hormones (e.g., Erythropoietin (EPO)), blood clotting protein (e.g., Factor IX), antibodies (e.g., anti-HER2 transturamab) and antigen binding fragments, protein receptors, fusion proteins, vaccines, cytokines (e.g., IL6), chemokines, growth factors, or blood factor proteins)) or nucleic acid (including, but not limited to, a recombinant virus or viral vector)), said method comprising culturing a population of mammalian cells (such as, for example, HEK293T cells, retinal pigment epithelial cells, or a mesenchymal stem cells), wherein the population of mammalian cells are genetically engineered to express two or more recombinant proteins (such as, for example, two or more recombinant proteins selected from the group consisting of STXBP1, SNAP23, SNAP25, SNAP91, VAMP2, VAMP7, BNIP1, GOSR1, BET1, SEX22B, YKT6, SCFD1, SAR1B, SEC24A, and STXBP1), wherein the population of mammalian cells are further genetically engineered to express the biomolecule; and, recovering said biomolecule of interest from the medium from said culture. In some embodiments, the population of mammalian cells are genetically engineered to express STXBP1, SEC24A, or SNAP23. In some embodiments, the population of mammalian cells are genetically engineered to express STXBP1 and SEC24A. In some embodiments, the population of mammalian cells are genetically engineered to express STXBP1 and SNAP23. In some embodiments, the population of mammalian cells are genetically engineered to express SEC24A and SNAP23. In some embodiments, the population of mammalian cells are genetically engineered to express STXBP1, SEC24A, and SNAP23.

In some embodiments, the two or more recombinant proteins synergize to increase production and/or secretion of the biomolecule of interest relative to a control culture of cells (such as, for example, a population of mammalian cells not genetically engineered to express two or more recombinant proteins).

In some embodiments, the two or more recombinant proteins synergize to increase production and/or secretion of two or more biomolecules of interest (such as, for example, two or more of recombinant proteins or polypeptides (including, but not limited to enzymes, prohormones (e.g., Chromogranin A), hormones (e.g., Erythropoietin (EPO)), blood clotting protein (e.g., Factor IX), antibodies (e.g., anti-HER2 transturamab) and antigen binding fragments, protein receptors, fusion proteins, vaccines, cytokines (e.g., IL6), chemokines, growth factors, or blood factor proteins)), nucleic acids (including, but not limited to, a recombinant virus or viral vector), or recombinant proteins or polypeptides and nucleic acids),

In some embodiments, the biomolecule of interest is tagged with a signal peptide. A signal peptide is a sequence on the N-terminus of a polypeptide or protein that signals secretion of the polypeptide or protein from a cell. In some embodiments, the signal peptide is about 5 to about 40 amino acids in length (such as about 5 to about 7, about 7 to about 10, about 10 to about 15, about 15 to about 20, about 20 to about 25, or about 25 to about 30, about 30 to about 35, or about 35 to about 40 amino acids in length). Exemplar signal peptides comprise:

| Signal Peptide | Signal Peptide Sequence | SEQ ID NO: |
|---|---|---|
| Interleukin-6 (IL6) | MNSFSTSAFGPVAFSLGLLLVLPAAFPAP | 1 |
| Interleukin-2 (IL2) | MQLLSCIALILALV | 2 |
| Gaussia luciferase | MGVKVLFALICIAVAEA | 3 |
| Chromogranin A (CHGA) | MRSAAVLALLLCAGQVTA | 4 |
| Chromogranin B (CHGF) | MQPTLLLSLLGAVGLAAVNS | 5 |
| Albumin (ALB) | MKWVTFISLLFLFSSAYS | 6 |
| Anti-HER2 heavy chain | MGWSLILLFLVAVATRVHS | 7 |
| Anti-HER2 light chain | MRVPAQLLGLLLLWLPGARC | 8 |
| Erythropoietin (EPO) | MGVHECPAWLWLLLSLLSLPLGLPVLG | 9 |
| Factor VIII(F8) | MQIELSTCFFLCLLRFCFS | 10 |
| Factor IX (F9) | MQRVNMIMAESPGLITICLLGYLLSAEC | 11 |
| Interferon alpha-2 (IFNA2) | MALTFALLVALLVLSCKSSCSVG | 12 |
| HMM + 38-secrecon (sec) | MWWRLWWLLLLLLLLWPMVWA | 13 |
| Vasopressin (AVP) | MPDTMLPACFLGLLAFSSA | 14 |
| IgG heavy chain | MDWTWRVFCLLAVTPGAH | 15 |
| (GenBank: AAA52897) | | |
| HMM + 34 | MRPTWAWWLFLVLLLALWAPARG | 16 |
| Neuroendocrine | MVSRMVSTMLSGLLFWLASGWTPAFA | 17 |
| protein 7B2 (SCG5) | | |
| Secretogranin II (SCG2) | MAEAKTHWLGAALSLIPLIFLISGAEA | 18 |
| Secretogranin III (SCG3) | MGFLGTGTWILVLVLPIQA | 19 |
| VGF nerve growth | MKALRLSASALFCLLLINGLGA | 20 |
| factor inducible | | |
| Trypsin (TRY1) w/propeptide | MNPLLILTFVAAALAAPFDDDDK | 21 |

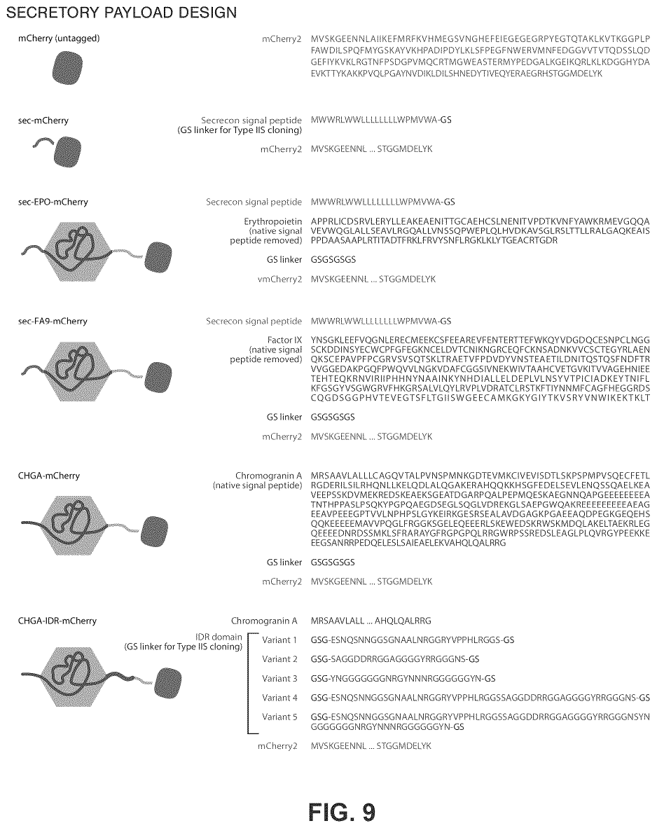

In embodiments, a linker (e.g., a flexible linker, such as GS(1-4) (SEQ ID NO: 22)) can be incorporated between the biomolecule of interest and the reporter domain. In embodiments, the can be replaced with a synthetic intrinsically disordered region (IDR) as the flexible linker between the biomolecule of interest and the reporter domain. Non-limiting examples of IDRs include Variant 1 through Variant 5; see, for example, FIG. 9) In regulated secretory granule exocytosis upon Ca2+ induction, including the IDR in the construct lowered basal secretory output compared to the design without while also showing a more dramatic granule based release from the IDR inclusive construct.

In some embodiments, the method comprises isolating or recovering said biomolecule of interest (e.g., a recombinant polypeptide and/or recombinant virus) from the medium from said culture. For example, the biomolecule of interest can be recovered from the cell culture medium directly or from spent medium that has been removed from the cell culture system. Once the medium has been removed from the cell culture system, it may be subjected to one or more processing steps to obtain the biomolecule of interest (e.g., a recombinant polypeptide and/or recombinant virus). Downstream processing steps include, without limitation, centrifugation and/or filtration to remove cells not previously withdrawn from the culture; affinity chromatography, hydrophobic interaction chromatography; ion-exchange chromatography; size exclusion chromatography; electrophoretic procedures (e.g., preparative isoelectric focusing (IEF), differential solubility (e.g., ammonium sulfate precipitation), extraction, and the like. See, generally, Scopes, Protein Purification, Springer-Verlag, New York, 1982; and Protein Purification, J.-C. Janson and Lars Ryden, editors, VCH Publishers, New York, 1989.

Any cell culture medium that supports cell growth and maintenance under the conditions of the invention may be used. Typically, the medium contains water, an osmolality regulator, a buffer, an energy source, amino acids, an inorganic or recombinant iron source, one or more synthetic or recombinant growth factors, vitamins, and cofactors.

In some embodiments, the method comprises transducing or transfecting the population of mammalian cells with one or more vectors expressing the two or more recombinant proteins (such as, for example, two or more recombinant proteins selected from the group consisting of STXBP1, SNAP23, SNAP25, SNAP91, VAMP2, VAMP7, BNIP1, GOSR1, BET1, SEX22B, YKT6, SCFD1, SAR1B, SEC24A, and STXBP1) and/or the biomolecule of interest (e.g., a recombinant polypeptide and/or recombinant virus). In some embodiments, the method comprises transducing or transfecting the population of mammalian cells with one or more vectors expressing STXBP1, SEC24A, or SNAP23. In some embodiments, the method comprises transducing or transfecting the population of mammalian cells with one or more vectors expressing STXBP1 and SEC24A. In some embodiments, the method comprises transducing or transfecting the population of mammalian cells with one or more vectors expressing STXBP1 and SNAP23. In some embodiments, the method comprises transducing or transfecting the population of mammalian cells with one or more vectors expressing SEC24A and SNAP23. In some embodiments, the method comprises transducing or transfecting the population of mammalian cells with one or more vectors expressing STXBP1, SEC24A, and SNAP23.

In some embodiments, the method comprises an in vitro method for producing a biomolecule of interest (such as, for example, a recombinant protein or polypeptide (including, but not limited to enzymes, prohormones (e.g., Chromogranin A), hormones (e.g., Erythropoietin (EPO)), blood clotting protein (e.g., Factor IX), antibodies (e.g., anti-HER2 transturamab) and antigen binding fragments, protein receptors, fusion proteins, vaccines, cytokines (e.g., IL6), chemokines, growth factors, or blood factor proteins)) or nucleic acid (including, but not limited to, a recombinant virus or viral vector)), said method comprising culturing a population of mammalian cells (such as, for example, HEK293T cells, retinal pigment epithelial cells, or a mesenchymal stem cells), wherein the population of mammalian cells are genetically engineered to express two or more recombinant proteins (such as, for example, two or more recombinant proteins selected from the group consisting of STXBP1, SNAP23, SNAP25, SNAP91, VAMP2, VAMP7, BNIP1, GOSR1, BET1, SEX22B, YKT6, SCFD1, SAR1B, SEC24A, and STXBP1), wherein the population of mammalian cells are further genetically engineered to express the biomolecule; and, recovering said biomolecule of interest from the medium from said culture.

For example, the in vitro method for producing a biomolecule of interest (such as, for example, a recombinant protein or polypeptide (including, but not limited to enzymes, prohormones (e.g., Chromogranin A), hormones (e.g., Erythropoietin (EPO)), blood clotting protein (e.g., Factor IX), antibodies (e.g., anti-HER2 transturamab) and antigen binding fragments, protein receptors, fusion proteins, vaccines, cytokines (e.g., IL6), chemokines, growth factors, or blood factor proteins)) or nucleic acid (including, but not limited to, a recombinant virus or viral vector)), said method comprising culturing a population of mammalian cells (such as, for example, HEK293T cells, retinal pigment epithelial cells, or a mesenchymal stem cells), wherein the population of mammalian cells are genetically engineered to express two or more recombinant proteins (such as, for example, two or more recombinant proteins selected from the group consisting of STXBP1, SNAP23, SNAP25, SNAP91, VAMP2, VAMP7, BNIP1, GOSR1, BET1, SEX22B, YKT6, SCFD1, SAR1B, SEC24A, and STXBP1), wherein the population of mammalian cells are further genetically engineered to express the biomolecule; and, recovering said biomolecule of interest from the medium from said culture, may be carried out in an appropriate culture unit (e.g, well, dish, plate, flask, tube or bottle) or bioreactor. The bioreactor can be of any size as long as it is useful for culturing cells, e.g., mammalian cells (such as, for example, HEK293T cells, retinal pigment epithelial cells, or a mesenchymal stem cells).

Also disclosed herein is a genetically engineered cell and/or a population of genetically engineered cells (including, but not limited to, mammalian cells (such as, for example, HEK293T cells, retinal pigment epithelial cells, or a mesenchymal stem cells)), wherein the genetically engineered cell and/or the population of genetically engineered cells are genetically engineered to express two or more recombinant proteins (such as, for example, two or more recombinant proteins selected from the group consisting of STXBP1, SNAP23, SNAP25, SNAP91, VAMP2, VAMP7, BNIP1, GOSR1, BET1, SEX22B, YKT6, SCFD1, SAR1B, SEC24A, and STXBP1). In some embodiments, the genetically engineered cell and/or the population of genetically engineered cells are genetically engineered to express STXBP1, SEC24A, or SNAP23. In some embodiments, the genetically engineered cell and/or the population of genetically engineered cells are genetically engineered to express STXBP1 and SEC24A. In some embodiments, the genetically engineered cell and/or the population of genetically engineered cells are genetically engineered to express STXBP1 and SNAP23. In some embodiments, the genetically engineered cell and/or the population of genetically engineered cells are genetically engineered to express SEC24A and SNAP23. In some embodiments, the genetically engineered cell and/or the population of genetically engineered cells are genetically engineered to express STXBP1, SEC24A, and SNAP23.

In some embodiments, the cells can be encapsulated in a variety of materials (such as, for example, natural polymers (for example, alginate, cellulose sulfate, hydrogels) or synthetic polymers (for example, polyacrylate and hydroxyethyl methacrylate-methacrylate copolymers).

In some embodiments, the genetically engineered cell and/or the population of genetically engineered cells are further engineered to secrete a biomolecule of interest (such as, for example, a recombinant protein or polypeptide (including, but not limited to enzymes, prohormones (e.g., Chromogranin A), hormones (e.g., Erythropoietin (EPO)), blood clotting protein (e.g., Factor IX), antibodies (e.g., anti-HER2 transturamab) and antigen binding fragments, protein receptors, fusion proteins, vaccines, cytokines (e.g., IL6), chemokines, growth factors, or blood factor proteins)) or nucleic acid (including, but not limited to, a recombinant virus or viral vector)).

In some embodiments, the two or more recombinant proteins and/or the biomolecule of interest can be expressed from one or more vectors or plasmids. For example, the biomolecule of interest can be expressed from one vector or one plasmid, and the two or more recombinant proteins can be expressed from one or more additional vectors or plasmids. In other embodiments, the biomolecule of interest and/or the two or more recombinant proteins can be expressed from a multi-gene vector or plasmid.

Further disclosed herein is a vector or plasmid (such as, for example, a lentiviral vector) encoding two or more recombinant proteins (such as, for example, two or more recombinant proteins selected from the group consisting of STXBP1, SNAP23, SNAP25, SNAP91, VAMP2, VAMP7, BNIP1, GOSR1, BET1, SEX22B, YKT6, SCFD1, SAR1B, SEC24A, and STXBP1). In some embodiments, the vector or plasmid encodes STXBP1, SEC24A, or SNAP23. In some embodiments, the vector or plasmid encodes STXBP1 and SEC24A. In some embodiments, the vector or plasmid encodes STXBP1 and SNAP23. In some embodiments, the vector or plasmid encodes SEC24A and SNAP23. In some embodiments, the vector or plasmid encodes STXBP1, SEC24A, and SNAP23.

In some embodiments, the vector or plasmid comprises one or more regulatory elements (such as, for example, a promoter (for example, NFAT-binding promoter or CMV promoter)).

Also disclosed herein is a cell or population of cells (including, but not limited to, mammalian cells (such as, for example, HEK293T cells, retinal pigment epithelial cells, or a mesenchymal stem cells)), comprising a vector or plasmid encoding two or more recombinant proteins (such as, for example, two or more recombinant proteins selected from the group consisting of STXBP1, SNAP23, SNAP25, SNAP91, VAMP2, VAMP7, BNIP1, GOSR1, BET1, SEX22B, YKT6, SCFD1, SAR1B, SEC24A, and STXBP1). In some embodiments, the vector or plasmid encodes STXBP1, SEC24A, and SNAP23.

In some embodiments, the cell or a population of cells can be encapsulated in a variety of materials (such as, for example, natural polymers (for example, alginate, cellulose sulfate, hydrogels) or synthetic polymers (for example, polyacrylate and hydroxyethyl methacrylate-methacrylate copolymers).

Engineered cell-based therapies secreting biomolecules of interest “on demand” benefit from fast responses. Accordingly, further disclosed herein are compositions and methods for regulated production of a biomolecule of interest, comprising a genetically engineered cell and/or a population of genetically engineered cells (including, but not limited to, mammalian cells (such as, for example, HEK293T cells, retinal pigment epithelial cells, or a mesenchymal stem cells)), wherein the genetically engineered cells and/or population of genetically engineered cells comprise a synthetic secretory pathway, wherein the synthetic secretory pathway comprises a recombinant membrane receptor configured to sense a signaling compound (such as, for example, cytosolic Ca²⁺), a recombinant calcium ion channel, and a recombinant secretion component (such as, for example, SYT7, STX4, RAB26, RAB27B, RAB3A, SYN1, SNAP91, STXBP1, RAB37, SYT1, RAB3C, and/or RIMS1, or any combination thereof), wherein the membrane receptor and the recombinant secretion component are operably linked by the recombinant calcium ion channel.

Reversible and toggle-able activation of secretion via calcium activation is useful for optimizing yields and long-term culturing in a batch context as well (i.e. the ability to turn it on and off easily is good for integrating into biologic production workflows that happen over longer periods of time).

In some embodiments, the regulated secretory pathway accumulates a reserve of secretory cargo until induce to secrete (such as, for example, by increase in cytosolic Ca²⁺). The Ca²⁺ sensor synaptotagmin (SYT)+ supporting SNARE proteins arrest secreting of granules until activated by Ca²⁺.

In some embodiments, the genetically engineered cells (including, but not limited to, mammalian cells (such as, for example, HEK293T cells, retinal pigment epithelial cells, or a mesenchymal stem cells)) can form dense core-regulated secretory granules.

In some embodiments, the cells can be encapsulated in a variety of materials (such as, for example, natural polymers (for example, alginate, cellulose sulfate, hydrogels) or synthetic polymers (for example, polyacrylate and hydroxyethyl methacrylate-methacrylate copolymers).

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The invention will be further described in the following examples, which do not limit the scope of the invention described in the claims.

### C. Examples

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

Mammalian cellular secretory machinery is an essential aspect of their use case as cell factories for biopharmaceutical production, as well as having implications on the performance of engineered cell-based therapeutic products. Essentially all recombinant drug products derived from mammalian cells must be secreted prior to purification steps, and many cell-based therapeutic platforms manage patient physiology via secretion-based signaling. Aspects of the invention are drawn towards compositions and methods for manipulating the mammalian secretory phenotype via overexpressed secretory factors to enhance bioproduction capacity, for example, in HEK293T cells. Our work demonstrates secretory pathway engineering to enhance bioproduction yields of biologic medicines and viral vectors for gene therapy, as well as augmenting the secretory productivity of cell types useful for therapeutics (e.g., mesenchymal stem cells and retinal pigment epithelial cells). For example, we demonstrate how secretory phenotype reprogramming can produce artificial neuroendocrine-like Ca²⁺-inducible regulated secretory responses in nonendocrine HEK293T cells. Finally, we engineer circuits for controlling Ca²⁺ channel activation with small-molecule inputs, as well as ongoing progress towards linking these circuits to our synthetic regulated secretory program outputs. Without wishing to be bound by theory, engineered regulated secretory cell circuits, especially those with reversible “sense-and-secrete” functionality and programmable and tunable inputs, can enhance the therapeutic capacity of cell lines.

### Example 2

Towards the goal of improving constitutive secretion to improve biopharmaceutical manufacturing, we conducted a bioinformatic search, comparing the expression of secretory pathway genes in HEK293 Ts compared to many cell types that specialize in secretion. Through this screen, we identified 24 candidate proteins that might alleviate bottlenecks through the ER-to-Golgi and Golgi-to-membrane trafficking. By evaluating the impact of overexpression of these proteins on the secretion of a fluorescent reporter, we identified 14 single genes that could boost secretion over 1.4×. We then tested these proteins for synergy by evaluating 78 ternary combinations, through which we found 39 combinations that improved secretion over 5×.

Through further screening, we found the combination of STXBP1, SEC24A, and SNAP23 to be the best performing combination. In the original ternary screen this combination gave us a 10.1× boost; consolidation of this program into a 3-gene plasmid yielded a 4.5× boost. We then tested this combination of proteins and found that it boosted production of high value compounds like trastuzumab (a monoclonal antibody also known as Herceptin) and lentivirus. Further, we found that the effect was conserved in therapeutically relevant cell types such as ARPEs and MSCs, both of which are used clinically for their secretory capabilities.

To improve regulated secretion, we developed a “sense-and-secrete” system. This system utilizes a membrane receptor to sense an extracellular biomarker, which then signals through a synthetic phosphorylation cascade within the cell and modulates a calcium channel. The subsequent calcium influx triggers the rapid release of pre-synthesized and pre-packaged cargo.

To accomplish this, we developed a secretory granule by fusing mCherry to Chromogranin A, a well-known secretory protein. We then compared HEK293 Ts to specialist secretory cells and identified 36 candidate proteins involved in regulated secretion then tested them in 321 combinations. We found that overexpression SYT7, STX4, RAB27A, and RAB27B improved regulated secretion by as much as 3.8×.

Next, we engineered a Ca²⁺ channel that could be opened via a protein-protein interaction by modifying the ORAI1 channel and the companion protein that activates it (the SOAR domain of STIM1). SOAR binding to ORAI1 induces intracellular Ca²⁺ flux, which activates SNARE-protein-mediated secretion. We engineered recruitment of SOAR to ORAI1 to be dependent on a small molecule dimerizer and as well as on a phosphorylation-dependent interaction. When either of these is triggered, there is an increase in Ca²⁺-dependent secretion. The phosphorylation-dependent channel can be implemented in a “sense-and-secrete” circuit that enables receptor ligand activation driven secretion in response to extracellular ligand sensing.

### Example 3

This technology can be used to improve yields with biomanufacturing platforms. For example, it can be employed to increase the specific productivity of HEK293 cells, thereby improving their commercial viability. It demonstrates the ability to enhance production yields across multiple product classes (e.g., antibodies, hormones, and viral vectors) and cell lines (e.g., HEK293T, MSCs, and ARPE19).

Additionally, the “sense-and-secrete” technology can significantly improve the utility of encapsulated cell therapies currently being used as implantable factory to produce biomolecules. Currently, encapsulated cell factories use constitutive secretion to produce biomolecules inside a host. This results in an “always on” state, the levels of which can be tuned by choosing promoters of differing strengths. Alternatively, these factories can use transcriptional circuits to produce the biomolecules only when triggered by an input. However, these circuits respond very slowly (on the orders of hours to days) and often take a long time to shut off. These circuit dynamics are not amenable to many biological processes in the human body, which rely on rapid release of pre-manufactured biomolecules that can be released all at once. Our “sense-and-secrete” circuit is a post-translational circuit, meaning all the components are pre-manufactured, and operates on a fast timescale (seconds to minutes), allowing the near-instantaneous delivery of therapeutics.

### Example 4

Other groups have demonstrated that overexpression of proteins from the secretory pathway can boost secretion of biopharmaceuticals or viral titer (STXBP2—Peng et al. 2010, SNAP-23 & VAMP8—Peng et al. 2011). We have shown the combination of STXBP1, SEC24A, and SNAP23 improves bioproduction 4.5×.

One key component of our “sense-and-secrete” circuit is the use of engineered SOAR (STIM1 Orai activating region) variants that triggers intracellular calcium influx through an engineered Orai1 channel. Other groups have modulated the activation of Orai1 using SOAR engineered with protease domains (Jazbec et al. 2022). Our circuit uses recruitment dependent interactions, relying on domains developed by our team (Yang et al. 2023).

The “sensor” part of our circuit also relies on receptor domains previously developed by our team (Yang et al. 2024).

The ability to form granules in constitutively secreting cells has previously been shown by other groups (Beuret et al. 2004, Stettler et al. 2009, Montero-Hadjadje et al. 2009). While the development of these granules has been shown before, we are the first group to move those granules through the secretory system and outside the cell.

In summary, we found a) a combination of transgenes that improve bioproduction and b) built a first in class “sense-and-secrete” system that can cause an increase in secreted cargo after binding a non-native extracellular ligand.

### Example 5

| SOAR-ZAP70_tSH2 | MAVELKRSEEQKLISEEDLLRSEEQKLISEEDLLRSEEQKLISEEDLL |
|---|---|
| | GNSSSWYAPEALQKWLQLTHEVEVQYYNIKKQNAEKQLLVAKEGAEKI |
| | KKKRNTLFGTFHVAHSSSLDDVDHKILTAKQALSEVTAALRERLHRWQ |
| | QIEILCGFQIVNNPGIHGSGSGSGSGSGSSGSGSGGSSGSGGSSSGGS |
| | GSGSGSSGGSGGGGSGGSSGSSSGGGGSPDPAAHLPFFYGSISRAEAE |
| | EHLKLAGMADGLFLLRQCLRSLGGYVLSLVHDVRFHHFPIERQLNGTY |
| | AIAGGKAHCGPAELCEFYSRDPDGLPCNLRKPCNRPSGLEPQPGVFDC |
| | LRDAMVRDYVRQTWKLEGEALEQAIISQAPQVEKLIATTAHERMPWYH |
| | SSLTREEAERKLYSGAQTDGKFLLRPRKEQGTYALSLIYGKTVYHYLI |
| | SQDKAGKYCIPEGTKFDTLWQLVEYLKLKADGLIYCLKEACPNSSAGS |
| | GSGSGSGSGSGSGSGSGSPDPAAHLPFFYGSISRAEAEEHLKLAGMAD |
| | GLFLLRQCLRSLGGYVLSLVHDVRFHHFPIERQLNGTYAIAGGKAHCG |
| | PAELCEFYSRDPDGLPCNLRKPCNRPSGLEPQPGVFDCLRDAMVRDYV |
| | RQTWKLEGEALEQAIISQAPQVEKLIATTAHERMPWYHSSLTREEAER |
| | KLYSGAQTDGKFLLRPRKEQGTYALSLIYGKTVYHYLISQDKAGKYCI |
| | PEGTKFDTLWQLVEYLKLKADGLIYCLKEACPNSSA* |
| | (SEQ ID NO: 23) |
| Orai (WT) | MHPEPAPPPSRSSPELPPSGGSTTSGSRRSRRRSGDGEPPGAPPPPPS |
| | AVTYPDWIGQSYSEVMSLNEHSMQALSWRKLYLSRAKLKASSRTSALL |
| | SGFAMVAMVEVQLDADHDYPPGLLIAFSACTTVLVAVHLFALMISTCI |
| | LPNIEAVSNVHNLNSVKESPHERMHRHIELAWAFSTVIGTLLFLAEVV |
| | LLCWVKFLPLKKQPGQPRPTSKPPASGAAANVSTSGITPGQAAAIAST |
| | TIMVPFGLIFIVFAVHFYRSLVSHKTDRQFQELNELAEFARLQDQLDH |
| | RGDHPLTPGSHYA (SEQ ID NO: 24) |
| ChgA-IDR-mCh2 | MRSAAVLALLLCAGQVTALPVNSPMNKGDTEVMKCIVEVISDTLSKPS |
| | PMPVSQECFETLRGDERILSILRHQNLLKELQDLALQGAKERAHQQKK |
| | HSGFEDELSEVLENQSSQAELKEAVEEPSSKDVMEKREDSKEAEKSGE |
| | ATDGARPQALPEPMQESKAEGNNQAPGEEEEEEEEATNTHPPASLPSQ |
| | KYPGPQAEGDSEGLSQGLVDREKGLSAEPGWQAKREEEEEEEEEAEAG |
| | EEAVPEEEGPTVVLNPHPSLGYKEIRKGESRSEALAVDGAGKPGAEEA |
| | QDPEGKGEQEHSQQKEEEEEMAVVPQGLFRGGKSGELEQEEERLSKEW |
| | EDSKRWSKMDQLAKELTAEKRLEGQEEEEDNRDSSMKLSFRARAYGFR |
| | GPGPQLRRGWRPSSREDSLEAGLPLQVRGYPEEKKEEEGSANRRPEDQ |
| | ELESLSAIEAELEKVAHQLQALRRGGSGESNQSNNGGSGNAALNRGGR |
| | YVPPHLRGGSGSMVSKGEENNLAIIKEFMRFKVHMEGSVNGHEFEIEG |
| | EGEGRPYEGTQTAKLKVTKGGPLPFAWDILSPQFMYGSKAYVKHPADI |
| | PDYLKLSFPEGFNWERVMNFEDGGVVTVTQDSSLQDGEFIYKVKLRGT |
| | NFPSDGPVMQCRTMGWEASTERMYPEDGALKGEIKQRLKLKDGGHYDA |
| | EVKTTYKAKKPVQLPGAYNVDIKLDILSHNEDYTIVEQYERAEGRHST |
| | GGMDELYK (SEQ ID NO: 25) |
| Rapalog induction circuits | |
| Myc-SOAR-FKBP | MVELKRSEEQKLISEEDLLRSEEQKLISEEDLLRSEEQKLISEEDLLG |
| | NSSSWYAPEALQKWLQLTHEVEVQYYNIKKQNAEKQLLVAKEGAEKIK |
| | KKRNTLFGTFHVAHSSSLDDVDHKILTAKQALSEVTAALRERLHRWQQ |
| | IEILCGFQIVNNPGIHGTGSGSGSGSGVQVETISPGDGRTFPKRGQTC |
| | VVHYTGMLEDGKKFDSSRDRNKPFKFMLGKQEVIRGWEEGVAQMSVGQ |
| | RAKLTISPDYAYGATGHPGIIPPHATLVFDVELLKLE (SEQ ID |
| | NO: 26) |
| Myc-SOAR-FRB | MVELKRSEEQKLISEEDLLRSEEQKLISEEDLLRSEEQKLISEEDLLG |
| | NSSSWYAPEALQKWLQLTHEVEVQYYNIKKQNAEKQLLVAKEGAEKIK |
| | KKRNTLFGTFHVAHSSSLDDVDHKILTAKQALSEVTAALRERLHRWQQ |
| | IEILCGFQIVNNPGIHGTGSGSGSGSILWHEMWHEGLEEASRLYFGER |
| | NVKGMFEVLEPLHAMMERGPQTLKETSFNQAYGRDLMEAQEWCRKYMK |
| | SGNVKDLLQAWDLYYHVFRRISK (SEQ ID NO: 27) |
| HA-Orai1-FKBP | MGNCSYPYDVPDYAGSYPYDVPDYAGSYPYDVPDYAGENSMHPEPAPP |
| | PSRSSPELPPSGGSTTSGSRRSRRRSGDGEPPGAPPPPPSAVTYPDWI |
| | GQSYSEVMSLNEHSMQALSWRKLYLSRAKLKASSRTSALLSGFAMVAM |
| | VEVQLDADHDYPPGLLIAFSACTTVLVAVHLFALMISTCILPNIEAVS |
| | NVHNLNSVKESPHERMHRHIELAWAFSTVIGTLLFLAEVVLLCWVKFL |
| | PLKKQPGQPRPTSKPPASGAAANVSTSGITPGQAAAIASTTIMVPFGL |
| | IFIVFAVHFYRSLVSHKTDRQFQELNELAEFARLQDQLDHRGDHPLTP |
| | GSHYAGTGSGSGSGSGVQVETISPGDGRTFPKRGQTCVVHYTGMLEDG |
| | KKFDSSRDRNKPFKFMLGKQEVIRGWEEGVAQMSVGQRAKLTISPDYA |
| | YGATGHPGIIPPHATLVEDVELLKLE (SEQ ID NO: 28) |
| SOAR mutations (core domain only) | |
| A369V | SSWYAPEALQKWLQLTHEVEVQYYNIKKQNVEKQLLVAKEGAEKIKKK |
| | RNTLFGTFHVAHSSSLDDVDHKILTAKQALSEVTAALRERLHRWQQIE |
| | ILCGFQIVNNPGIH (SEQ ID NO: 29) |
| A369D | SSWYAPEALQKWLQLTHEVEVQYYNIKKQNDEKQLLVAKEGAEKIKKK |
| | RNTLFGTFHVAHSSSLDDVDHKILTAKQALSEVTAALRERLHRWQQIE |
| | ILCGFQIVNNPGIH (SEQ ID NO: 30) |
| A369K | SSWYAPEALQKWLQLTHEVEVQYYNIKKQNKEKQLLVAKEGAEKIKKK |
| | RNTLFGTFHVAHSSSLDDVDHKILTAKQALSEVTAALRERLHRWQQIE |
| | ILCGFQIVNNPGIH (SEQ ID NO: 31) |
| A369S | SSWYAPEALQKWLQLTHEVEVQYYNIKKQNSEKQLLVAKEGAEKIKKK |
| | RNTLFGTFHVAHSSSLDDVDHKILTAKQALSEVTAALRERLHRWQQIE |
| | ILCGFQIVNNPGIH (SEQ ID NO: 32) |
| A369G | SSWYAPEALQKWLQLTHEVEVQYYNIKKQNGEKQLLVAKEGAEKIKKK |
| | RNTLFGTFHVAHSSSLDDVDHKILTAKQALSEVTAALRERLHRWQQIE |
| | ILCGFQIVNNPGIH (SEQ ID NO: 33) |
| L373A | SSWYAPEALQKWLQLTHEVEVQYYNIKKQNAEKQALVAKEGAEKIKKK |
| | RNTLFGTFHVAHSSSLDDVDHKILTAKQALSEVTAALRERLHRWQQIE |
| | ILCGFQIVNNPGIH (SEQ ID NO: 34) |
| L373V | SSWYAPEALQKWLQLTHEVEVQYYNIKKQNAEKQVLVAKEGAEKIKKK |
| | RNTLFGTFHVAHSSSLDDVDHKILTAKQALSEVTAALRERLHRWQQIE |
| | ILCGFQIVNNPGIH (SEQ ID NO: 35) |
| L373S | SSWYAPEALQKWLQLTHEVEVQYYNIKKQNAEKQSLVAKEGAEKIKKK |
| | RNTLFGTFHVAHSSSLDDVDHKILTAKQALSEVTAALRERLHRWQQIE |
| | ILCGFQIVNNPGIH (SEQ ID NO: 36) |
| A376D | SSWYAPEALQKWLQLTHEVEVQYYNIKKQNAEKQLLVDKEGAEKIKKK |
| | RNTLFGTFHVAHSSSLDDVDHKILTAKQALSEVTAALRERLHRWQQIE |
| | ILCGFQIVNNPGIH (SEQ ID NO: 37) |
| A376K | SSWYAPEALQKWLQLTHEVEVQYYNIKKQNAEKQLLVKKEGAEKIKKK |
| | RNTLFGTFHVAHSSSLDDVDHKILTAKQALSEVTAALRERLHRWQQIE |
| | ILCGFQIVNNPGIH (SEQ ID NO: 38) |
| A376S | SSWYAPEALQKWLQLTHEVEVQYYNIKKQNAEKQLLVSKEGAEKIKKK |
| | RNTLFGTFHVAHSSSLDDVDHKILTAKQALSEVTAALRERLHRWQQIE |
| | ILCGFQIVNNPGIH (SEQ ID NO: 39) |
| A376G | SSWYAPEALQKWLQLTHEVEVQYYNIKKQNAEKQLLVGKEGAEKIKKK |
| | RNTLFGTFHVAHSSSLDDVDHKILTAKQALSEVTAALRERLHRWQQIE |
| | ILCGFQIVNNPGIH (SEQ ID NO: 40) |
| A380D | SSWYAPEALQKWLQLTHEVEVQYYNIKKQNAEKQLLVAKEGDEKIKKK |
| | RNTLFGTFHVAHSSSLDDVDHKILTAKQALSEVTAALRERLHRWQQIE |
| | ILCGFQIVNNPGIH (SEQ ID NO: 41) |
| A380K | SSWYAPEALQKWLQLTHEVEVQYYNIKKQNAEKQLLVAKEGKEKIKKK |
| | RNTLFGTFHVAHSSSLDDVDHKILTAKQALSEVTAALRERLHRWQQIE |
| | ILCGFQIVNNPGIH (SEQ ID NO: 42) |
| A380S | SSWYAPEALQKWLQLTHEVEVQYYNIKKQNAEKQLLVAKEGSEKIKKK |
| | RNTLFGTFHVAHSSSLDDVDHKILTAKQALSEVTAALRERLHRWQQIE |
| | ILCGFQIVNNPGIH (SEQ ID NO: 43) |
| A380G | SSWYAPEALQKWLQLTHEVEVQYYNIKKQNAEKQLLVAKEGGEKIKKK |
| | RNTLFGTFHVAHSSSLDDVDHKILTAKQALSEVTAALRERLHRWQQIE |
| | ILCGFQIVNNPGIH (SEQ ID NO: 44) |
| Reporter sequence designs | |
| sec-mCh2 | MWWRLWWLLLLLLLLWPMVWAGSMVSKGEENNLAIIKEFMRFKVHMEG |
| | SVNGHEFEIEGEGEGRPYEGTQTAKLKVTKGGPLPFAWDILSPQFMYG |
| | SKAYVKHPADIPDYLKLSFPEGFNWERVMNFEDGGVVTVTQDSSLQDG |
| | EFIYKVKLRGTNFPSDGPVMQCRTMGWEASTERMYPEDGALKGEIKQR |
| | LKLKDGGHYDAEVKTTYKAKKPVQLPGAYNVDIKLDILSHNEDYTIVE |
| | QYERAEGRHSTGGMDELYK (SEQ ID NO: 45) |
| sec-EPO-mCh2 | MWWRLWWLLLLLLLLWPMVWAGSAPPRLICDSRVLERYLLEAKEAENI |
| | TTGCAEHCSLNENITVPDTKVNFYAWKRMEVGQQAVEVWQGLALLSEA |
| | VLRGQALLVNSSQPWEPLQLHVDKAVSGLRSLTTLLRALGAQKEAISP |
| | PDAASAAPLRTITADTFRKLFRVYSNFLRGKLKLYTGEACRTGDRGSG |
| | SGSGSMVSKGEENNLAIIKEFMRFKVHMEGSVNGHEFEIEGEGEGRPY |
| | EGTQTAKLKVTKGGPLPFAWDILSPQFMYGSKAYVKHPADIPDYLKLS |
| | FPEGFNWERVMNFEDGGVVTVTQDSSLQDGEFIYKVKLRGTNFPSDGP |
| | VMQCRTMGWEASTERMYPEDGALKGEIKQRLKLKDGGHYDAEVKTTYK |
| | AKKPVQLPGAYNVDIKLDILSHNEDYTIVEQYERAEGRHSTGGMDELY |
| | K (SEQ ID NO: 46) |
| sec-FA9-mCh2 | MWWRLWWLLLLLLLLWPMVWAGSYNSGKLEEFVQGNLERECMEEKCSF |
| | EEAREVFENTERTTEFWKQYVDGDQCESNPCLNGGSCKDDINSYECWC |
| | PFGFEGKNCELDVTCNIKNGRCEQFCKNSADNKVVCSCTEGYRLAENQ |
| | KSCEPAVPFPCGRVSVSQTSKLTRAETVFPDVDYVNSTEAETILDNIT |
| | QSTQSFNDFTRVVGGEDAKPGQFPWQVVLNGKVDAFCGGSIVNEKWIV |
| | TAAHCVETGVKITVVAGEHNIEETEHTEQKRNVIRIIPHHNYNAAINK |
| | YNHDIALLELDEPLVLNSYVTPICIADKEYTNIFLKFGSGYVSGWGRV |
| | FHKGRSALVLQYLRVPLVDRATCLRSTKFTIYNNMFCAGFHEGGRDSC |
| | QGDSGGPHVTEVEGTSFLTGIISWGEECAMKGKYGIYTKVSRYVNWIK |
| | EKTKLTGSGSGSGSMVSKGEENNLAIIKEFMRFKVHMEGSVNGHEFEI |
| | EGEGEGRPYEGTQTAKLKVTKGGPLPFAWDILSPQFMYGSKAYVKHPA |
| | DIPDYLKLSFPEGFNWERVMNFEDGGVVTVTQDSSLQDGEFIYKVKLR |
| | GTNFPSDGPVMQCRTMGWEASTERMYPEDGALKGEIKQRLKLKDGGHY |
| | DAEVKTTYKAKKPVQLPGAYNVDIKLDILSHNEDYTIVEQYERAEGRH |
| | STGGMDELYK (SEQ ID NO: 47) |
| ChgA-mCh2 | MRSAAVLALLLCAGQVTALPVNSPMNKGDTEVMKCIVEVISDTLSKPS |
| | PMPVSQECFETLRGDERILSILRHQNLLKELQDLALQGAKERAHQQKK |
| | HSGFEDELSEVLENQSSQAELKEAVEEPSSKDVMEKREDSKEAEKSGE |
| | ATDGARPQALPEPMQESKAEGNNQAPGEEEEEEEEATNTHPPASLPSQ |
| | KYPGPQAEGDSEGLSQGLVDREKGLSAEPGWQAKREEEEEEEEEAEAG |
| | EEAVPEEEGPTVVLNPHPSLGYKEIRKGESRSEALAVDGAGKPGAEEA |
| | QDPEGKGEQEHSQQKEEEEEMAVVPQGLFRGGKSGELEQEEERLSKEW |
| | EDSKRWSKMDQLAKELTAEKRLEGQEEEEDNRDSSMKLSFRARAYGER |
| | GPGPQLRRGWRPSSREDSLEAGLPLQVRGYPEEKKEEEGSANRRPEDQ |
| | ELESLSAIEAELEKVAHQLQALRRGGSGSGSGSMVSKGEENNLAIIKE |
| | FMRFKVHMEGSVNGHEFEIEGEGEGRPYEGTQTAKLKVTKGGPLPFAW |
| | DILSPQFMYGSKAYVKHPADIPDYLKLSFPEGENWERVMNFEDGGVVT |
| | VTQDSSLQDGEFIYKVKLRGTNFPSDGPVMQCRTMGWEASTERMYPED |
| | GALKGEIKQRLKLKDGGHYDAEVKTTYKAKKPVQLPGAYNVDIKLDIL |
| | SHNEDYTIVEQYERAEGRHSTGGMDELYK (SEQ ID NO: 48) |
| ChgA-IDR2-mCh2 | MRSAAVLALLLCAGQVTALPVNSPMNKGDTEVMKCIVEVISDTLSKPS |
| | PMPVSQECFETLRGDERILSILRHQNLLKELQDLALQGAKERAHQQKK |
| | HSGFEDELSEVLENQSSQAELKEAVEEPSSKDVMEKREDSKEAEKSGE |
| | ATDGARPQALPEPMQESKAEGNNQAPGEEEEEEEEATNTHPPASLPSQ |
| | KYPGPQAEGDSEGLSQGLVDREKGLSAEPGWQAKREEEEEEEEEAEAG |
| | EEAVPEEEGPTVVLNPHPSLGYKEIRKGESRSEALAVDGAGKPGAEEA |
| | QDPEGKGEQEHSQQKEEEEEMAVVPQGLFRGGKSGELEQEEERLSKEW |
| | EDSKRWSKMDQLAKELTAEKRLEGQEEEEDNRDSSMKLSFRARAYGER |
| | GPGPQLRRGWRPSSREDSLEAGLPLQVRGYPEEKKEEEGSANRRPEDQ |
| | ELESLSAIEAELEKVAHQLQALRRGGSGSAGGDDRRGGAGGGGYRRGG |
| | GNSGSMVSKGEENNLAIIKEFMRFKVHMEGSVNGHEFEIEGEGEGRPY |
| | EGTQTAKLKVTKGGPLPFAWDILSPQFMYGSKAYVKHPADIPDYLKLS |
| | FPEGFNWERVMNFEDGGVVTVTQDSSLQDGEFIYKVKLRGTNFPSDGP |
| | VMQCRTMGWEASTERMYPEDGALKGEIKQRLKLKDGGHYDAEVKTTYK |
| | AKKPVQLPGAYNVDIKLDILSHNEDYTIVEQYERAEGRHSTGGMDELY |
| | K (SEQ ID NO: 49) |
| ChgA-IDR3-mCh2 | MRSAAVLALLLCAGQVTALPVNSPMNKGDTEVMKCIVEVISDTLSKPS |
| | PMPVSQECFETLRGDERILSILRHQNLLKELQDLALQGAKERAHQQKK |
| | HSGFEDELSEVLENQSSQAELKEAVEEPSSKDVMEKREDSKEAEKSGE |
| | ATDGARPQALPEPMQESKAEGNNQAPGEEEEEEEEATNTHPPASLPSQ |
| | KYPGPQAEGDSEGLSQGLVDREKGLSAEPGWQAKREEEEEEEEEAEAG |
| | EEAVPEEEGPTVVLNPHPSLGYKEIRKGESRSEALAVDGAGKPGAEEA |
| | QDPEGKGEQEHSQQKEEEEEMAVVPQGLFRGGKSGELEQEEERLSKEW |
| | EDSKRWSKMDQLAKELTAEKRLEGQEEEEDNRDSSMKLSFRARAYGER |
| | GPGPQLRRGWRPSSREDSLEAGLPLQVRGYPEEKKEEEGSANRRPEDQ |
| | ELESLSAIEAELEKVAHQLQALRRGGSGYNGGGGGGGNRGYNNNRGGG |
| | GGGYNGSMVSKGEENNLAIIKEFMRFKVHMEGSVNGHEFEIEGEGEGR |
| | PYEGTQTAKLKVTKGGPLPFAWDILSPQFMYGSKAYVKHPADIPDYLK |
| | LSFPEGFNWERVMNFEDGGVVTVTQDSSLQDGEFIYKVKLRGTNFPSD |
| | GPVMQCRTMGWEASTERMYPEDGALKGEIKQRLKLKDGGHYDAEVKTT |
| | YKAKKPVQLPGAYNVDIKLDILSHNEDYTIVEQYERAEGRHSTGGMDE |
| | LYK (SEQ ID NO: 50) |
| ChgA-IDR4-mCh2 | MRSAAVLALLLCAGQVTALPVNSPMNKGDTEVMKCIVEVISDTLSKPS |
| | PMPVSQECFETLRGDERILSILRHQNLLKELQDLALQGAKERAHQQKK |
| | HSGFEDELSEVLENQSSQAELKEAVEEPSSKDVMEKREDSKEAEKSGE |
| | ATDGARPQALPEPMQESKAEGNNQAPGEEEEEEEEATNTHPPASLPSQ |
| | KYPGPQAEGDSEGLSQGLVDREKGLSAEPGWQAKREEEEEEEEEAEAG |
| | EEAVPEEEGPTVVLNPHPSLGYKEIRKGESRSEALAVDGAGKPGAEEA |
| | QDPEGKGEQEHSQQKEEEEEMAVVPQGLERGGKSGELEQEEERLSKEW |
| | EDSKRWSKMDQLAKELTAEKRLEGQEEEEDNRDSSMKLSFRARAYGER |
| | GPGPQLRRGWRPSSREDSLEAGLPLQVRGYPEEKKEEEGSANRRPEDQ |
| | ELESLSAIEAELEKVAHQLQALRRGGSGESNQSNNGGSGNAALNRGGR |
| | YVPPHLRGGSSAGGDDRRGGAGGGGYRRGGGNSGSMVSKGEENNLAII |
| | KEFMRFKVHMEGSVNGHEFEIEGEGEGRPYEGTQTAKLKVTKGGPLPF |
| | AWDILSPQFMYGSKAYVKHPADIPDYLKLSFPEGENWERVMNFEDGGV |
| | VTVTQDSSLQDGEFIYKVKLRGTNFPSDGPVMQCRTMGWEASTERMYP |
| | EDGALKGEIKQRLKLKDGGHYDAEVKTTYKAKKPVQLPGAYNVDIKLD |
| | ILSHNEDYTIVEQYERAEGRHSTGGMDELYK (SEQ ID NO: 51) |
| ChgA-IDR5-mCh2 | MRSAAVLALLLCAGQVTALPVNSPMNKGDTEVMKCIVEVISDTLSKPS |
| | PMPVSQECFETLRGDERILSILRHQNLLKELQDLALQGAKERAHQQKK |
| | HSGFEDELSEVLENQSSQAELKEAVEEPSSKDVMEKREDSKEAEKSGE |
| | ATDGARPQALPEPMQESKAEGNNQAPGEEEEEEEEATNTHPPASLPSQ |
| | KYPGPQAEGDSEGLSQGLVDREKGLSAEPGWQAKREEEEEEEEEAEAG |
| | EEAVPEEEGPTVVLNPHPSLGYKEIRKGESRSEALAVDGAGKPGAEEA |
| | QDPEGKGEQEHSQQKEEEEEMAVVPQGLFRGGKSGELEQEEERLSKEW |
| | EDSKRWSKMDQLAKELTAEKRLEGQEEEEDNRDSSMKLSFRARAYGER |
| | GPGPQLRRGWRPSSREDSLEAGLPLQVRGYPEEKKEEEGSANRRPEDQ |
| | ELESLSAIEAELEKVAHQLQALRRGGSGESNQSNNGGSGNAALNRGGR |
| | YVPPHLRGGSSAGGDDRRGGAGGGGYRRGGGNSYNGGGGGGGNRGYNN |
| | NRGGGGGGYNGSMVSKGEENNLAIIKEFMRFKVHMEGSVNGHEFEIEG |
| | EGEGRPYEGTQTAKLKVTKGGPLPFAWDILSPQFMYGSKAYVKHPADI |
| | PDYLKLSFPEGFNWERVMNFEDGGVVTVTQDSSLQDGEFIYKVKLRGT |
| | NFPSDGPVMQCRTMGWEASTERMYPEDGALKGEIKQRLKLKDGGHYDA |
| | EVKTTYKAKKPVQLPGAYNVDIKLDILSHNEDYTIVEQYERAEGRHST |
| | GGMDELYK (SEQ ID NO: 52) |

### Example 6—Synthetic Manipulation of Mammalian Secretory Phenotype

### Abstract

Engineering robust activity and control of secretion in human cells remains a challenge in recombinant protein bioproduction and cell-based therapeutics. While cell lines commonly for biotechnology applications have advantages such as genetic manipulability and culture scalability, they lack strong secretory or regulatable secretory profiles observed in native cell types that specialize in secretory function. In this example, we demonstrate that ectopic expression of multi-gene programs enhanced secretory output of diverse protein products in a commonly used human cell line. We also identified programs that enable Ca²⁺-regulated secretion. Finally, we used these the programs to create a synthetic circuit that secretes protein payloads in response to extracellular ligand sensing. Aspects of the invention provides a versatile framework for reshaping mammalian secretory function, including boosting biopharmaceutical production and creating post-translational secretory programs with custom inputs and outputs that can improve cell-based therapeutics.

### INTRODUCTION

In recent decades, mammalian cell lines have become vital to the production of an ever-growing list of advanced biologic medicines, including immunoglobulin-based therapeutics, soluble immunoregulatory ligands, and catalytic biologics. To date, strategies for improving production have primarily focused on maximizing cell health, culture density, and productivity by optimizing media formulations and bioreactor control strategies. Efforts to improve the secretory output are constrained by the specific secreted product and cell line contexts, with limited generalizability. There is a growing need for enhancements to human workhorse cell lines (e.g., HEK293 cells) critical for synthesis of specific therapeutic products such as viral vectors for gene therapies.

Genetic modifications to relieve biosynthesis bottlenecks can enhance cellular secretory production. Examples include overexpression or knockdown of factors involved in protein folding in the endoplasmic reticulum (ER), glycosylation in the ER and Golgi apparatus, and the unfolded protein response (UPR). Modulating the expression of factors that regulate cell metabolism increases bioproduction capacity by improving cell health, division rate, or specific productivity. Additionally, overexpression of different factors like heat shock proteins, ER chaperones, XBP1, can improve cultured cell health, boost bioproduction, or improve product quality; however, the core cellular secretory machinery and its factors remain underexplored as engineering targets. Thus, multi-factor synergies to boost bioproduction and inducible expression of these factors for dynamic optimization of cell factory behavior also remain underexplored.

Implantable cell-based therapeutic systems might also benefit from secretory phenotype engineering. Because therapeutically useful primary cell lines and their stem-cell derived counterparts often suffer from cell culture scale-up issues and product inconsistency, an alternative approach is to engineer cell circuits that mimic a desired therapeutic phenotype. However, the therapeutic efficacy of inducible secretory circuits is typically limited by the transcriptional timescale, rendering them unable to enact a fast-acting secretory response to physiological signals. A number of synthetic secretory circuits mitigate the timescale limitations of current synthetic biological circuits by stockpiling secretory cargo in the endoplasmic reticulum (ER), inducing secretion only upon post-translationally induced proteolytic removal of ER-retention tags from the cargos; however, timescale limitations may be introduced when using these methods by protease circuit kinetics and transport of liberated cargos from the ER to the plasma membrane. A synthetic post-translational regulated secretory response that resembles that of neuroendocrine primary cells, implemented in a cell line with facile culture requirements, might be able to overcome these limitations and expand the application space of synthetic biological circuits for engineering cell-based therapies. However, minimal research has been conducted to explore the possibility of altering the default secretory phenotype of nominally constitutive-only secretory cells, even as evidence emerges that ectopic expression of regulated secretory factors in these cells can elicit regulated-like secretory behaviors.

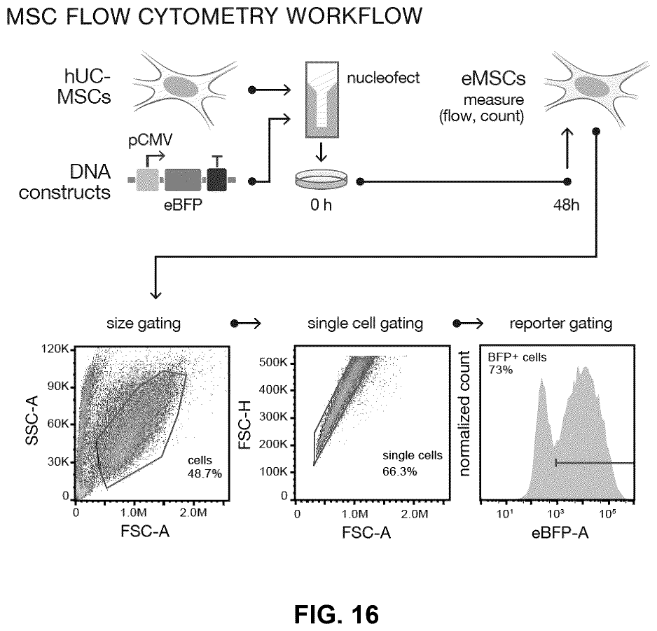

Here, we validated that the mammalian secretory phenotype possesses significant plasticity, and can be manipulated through targeted overexpression of secretory pathway factors. We identified factors that provide broad coverage of core constitutive and regulated secretion, cross-referencing factors featured in canonical secretory models against bioinformatic analyses of the human transcriptome across different cell lines. HEK293T cells transfected with specific combinations of these factors exhibited synergistic interactions that we could exploit to boost secretory bioproduction or imparted a Ca²⁺-responsive regulated-like secretory phenotype. Bioproduction-boosting synergies were employed in HEK cells to enhance secretion of fluorescent secretion reporters, fluorescently tagged recombinant therapeutic proteins, and lentiviral vectors, as well as cytokine production by mesenchymal stem cells (MSCs). To interface with our engineered regulated secretory phenotype, we developed rapamycin-gated Ca²⁺ channel circuit modules using fusion proteins based on the STIM-Orai activation region (SOAR) domain of STIM1 and Orai1, a system originally evolved to restore depleted Ca²⁺ stores in the ER (FIG. 16). In doing so, we constructed a post-translational and rapid sense-and-secrete cellular circuit inspired by neuroendocrine regulated secretion that allows for modularization of inputs and outputs. Without wishing to be bound by theory, this design can augment the therapeutic capacity of implantable cell lines possessing facile cell culture requirements with synthetic programmable regulated secretory circuits that mimic the operational timescales of neuroendocrine cell types.

### Results

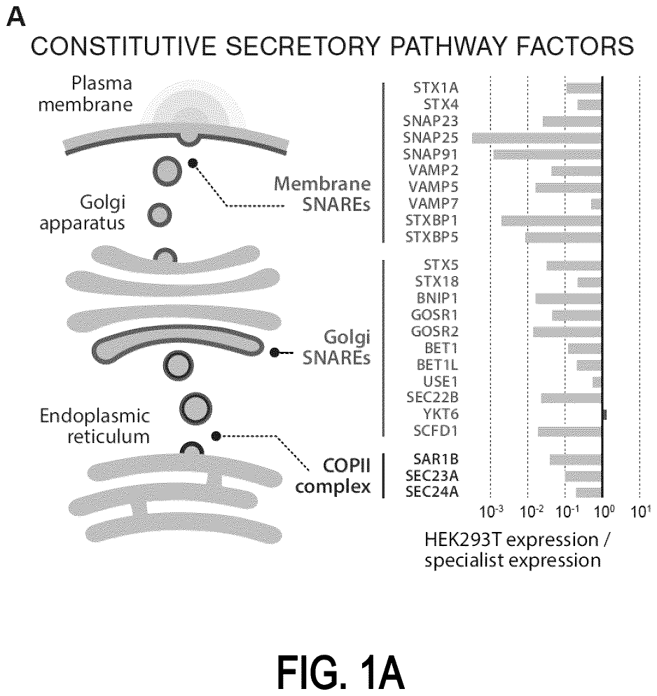

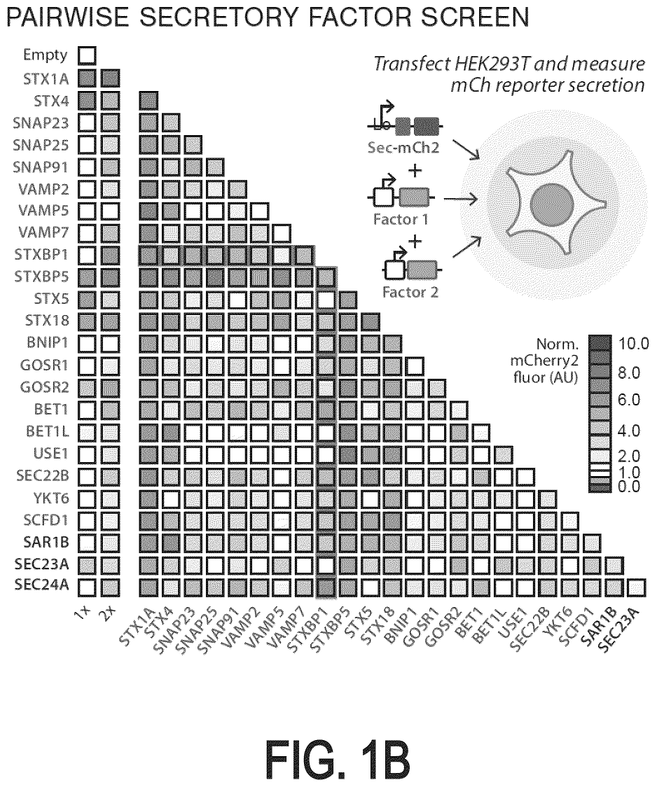

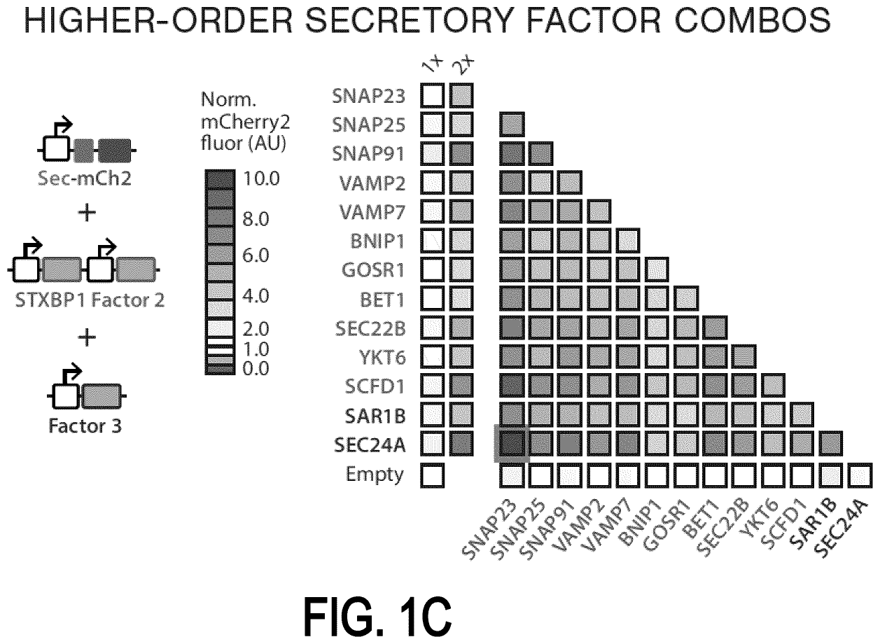

To test the possibility that the bioproduction capacity of mammalian cells could be manipulated through ectopic expression of secretory pathway factors, we identified a panel of proteins canonically involved in different stages of the secretory process (FIG. 1A). Secreted proteins contain N-terminal signal peptide tags that are recognized by a signal recognition particle (SRP), which directs further translation into the ER prior to cleavage of the tag from the core protein. In the ER, secretory proteins undergo chaperone-mediated folding, as well as post-translational modifications glycosylation by glycosyltransferases, after which they are packaged into coat protein complex II(COPII) vesicles for anterograde transport to the Golgi. Fusion to the cis-Golgi face is mediated by ER-Golgi SNAREs, after which secretory proteins undergo further processing within the Golgi apparatus before being exported in cargo-containing vesicles from the trans-Golgi face. Mature secretory vesicles then undergo exocytosis upon fusion with interaction with membrane SNARE proteins at the plasma membrane.

The COPII, ER-Golgi SNARE, and membrane SNARE proteins comprise a core secretory machinery wherein these parts must work together to transport cargos from the ER to the Golgi complex and subsequently the plasma membrane for exocytosis (FIG. 1A). Guided by this model, we generated a list of candidate secretory proteins for screening wherein we selected examples to represent each key protein family along the core secretory export pathway.
For the COPII complex, we chose SAR1B, SEC23A, and SEC24A to map to the inner COPII vesicle Sar1-Sec23-Sec24 lattice; high redundancy exists between isoforms for these 3 protein families.
We selected a representative set of ER-Golgi SNARE complex proteins for screening, in particular prominent examples of the Qa-SNARE (STX5, STX18), Qb-SNARE (BNIP, GOSR1, GOSR2), Qc-SNARE (BET1, BET1L, USE1), R-SNARE (SEC22B, YKT6), and Sec1/Munc18 (SCFD1) complex components.
For coverage of the membrane secretory SNAREs, we chose several well-characterized examples of membrane syntaxins (STX1A, STX4), soluble NSF attachment proteins (SNAP23, SNAP25, SNAP91), vesicle-associated membrane proteins (VAMP2, VAMP5, VAMP7) and Sec1/Munc18 proteins (STXBP1, STXBP5). Comparison of HEK293T expression of this protein selection to a set of four cell types (plasma cells, hepatocytes, B-cells, and pancreatic endocrine cells) well-known as highly-productive professional secretors revealed substantial enrichment in several of our selected factors, particularly for membrane SNAREs and COPII complex proteins.

With our screen, we sought to validate that targeted overexpression of specific combinations of these factors would reveal synergistic interactions with the capacity to boost production of recombinant biologic medicines (e.g., therapeutic proteins and viral vectors).

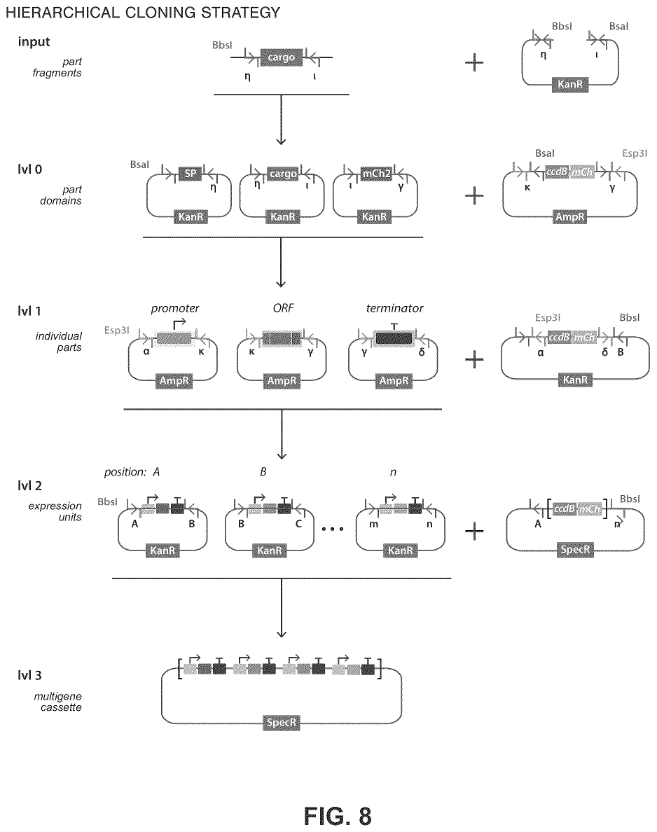

To assay secretion capacity, we developed a fluorescence-based secretory reporter with improved experimental throughput and precision compared with enzyme-based reporters (e.g., secreted alkaline phosphatase and luciferase) (FIG. 8A). We fused a synthetic signal peptide to mCherry2 (sec-mCh2), which was selected for its resistance to oxidation in the ER compared to oligomerization-prone GFPs. We found that this reporter design secreted efficiently with low background signal, limited intracellular retention, and high precision and repeatability (FIG. 8B). We also showed that the mCh2 reporter could be used to tag high value secretory proteins such as erythropoietin (EPO), clotting factor 9 (FA9), and chromogranin A (CHGA) with C-terminal mCh2 tags (FIG. 8C).

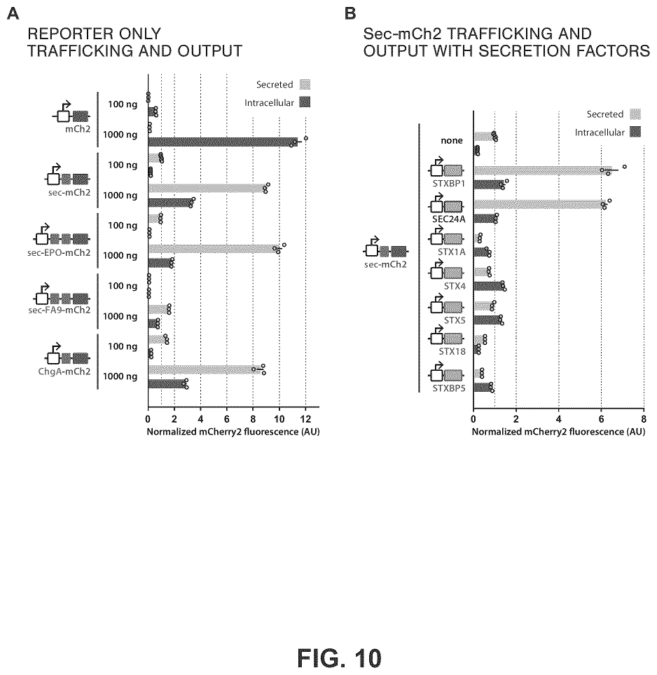

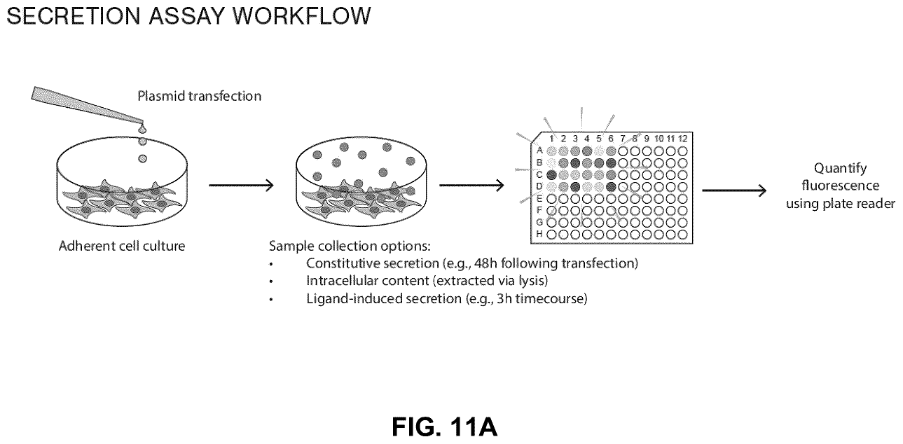

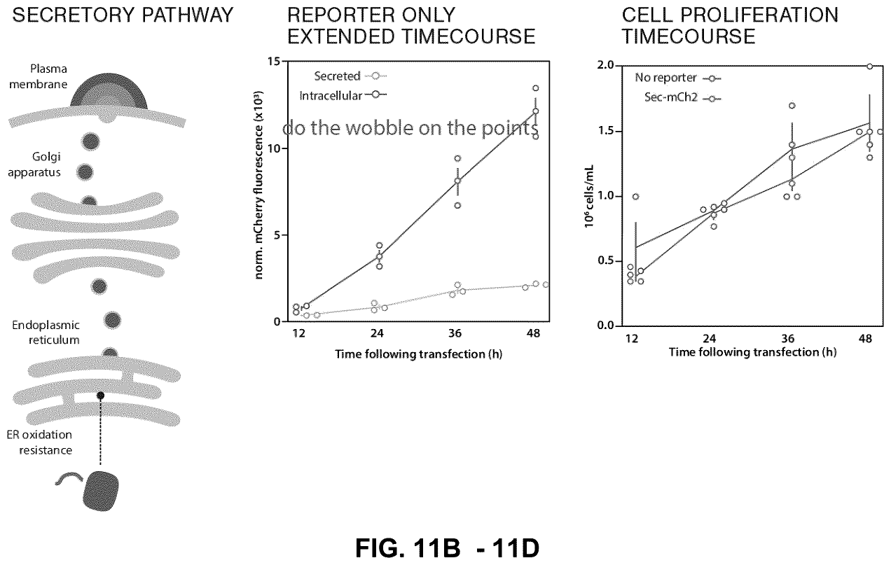

We transfected HEK293T cells with sec-mCh2 and combinations of secretory factors to assess their effect on secretion. Overexpression of specific secretory factors such as Munc18-1 (STXBP1) and synaptosome-associated proteins (SNAP) enhanced reporter secretion, whereas others, especially syntaxins (STX) and tomosyn (STXBP5), substantially reduced secretory output (FIG. 1B). Both secretion-boosting and -reducing factors tended to increase intracellular content of reporter, indicating increased secretory machinery throughput in the former case and worsening of secretory bottlenecking in the latter (FIG. 8). Without wishing to be bound by theory, any debottlenecking achieved by overexpressing a single factor will shift the secretion rate-limiting steps to other parts of the pathway. By co-transfecting HEK293T cells to overexpress combinations of two secretory factors, we uncovered multiple synergies enhancing bioproduction significantly greater than gains from single-factor transfections (FIG. 11). To quantify these synergies, we computed the interaction coefficient (IC), where f(A+B) is the secretory response to co-transfection of factors A and B, and the single-factor secretory response f(B)≥f(A). The strongest synergies were observed with Munc18-1 (STXBP1) co-transfected with other secretion-boosting factors such as SNAP23 and SEC24A. Notably, STX4 and STX5 each had reduced secretion-inhibiting effects when transfected at higher (2×) doses, paradoxically generating relatively large positive IC values despite reducing secretory production. We found selected binary synergies (e.g., STXBP1+SEC24A) to remain robust even as the secrecon tag is swapped for more potent signal peptides (FIG. 9) and when alternative reporter designs such as EPO-mCh2, FA9-mCh2, and CHGA-mCh2 are evaluated (FIG. 10). The results of this screen revealed the existence of synergies not only within a single category of secretory pathway factors, such as between the membrane SNAREs STXBP1 and SNAP23, but also across categories as seen with STXBP1 and the COPII complex protein SEC24A.

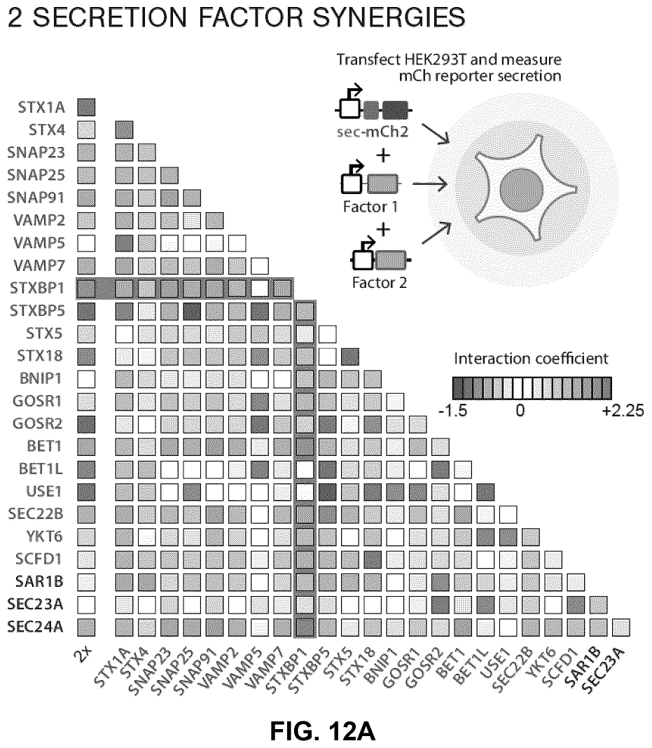

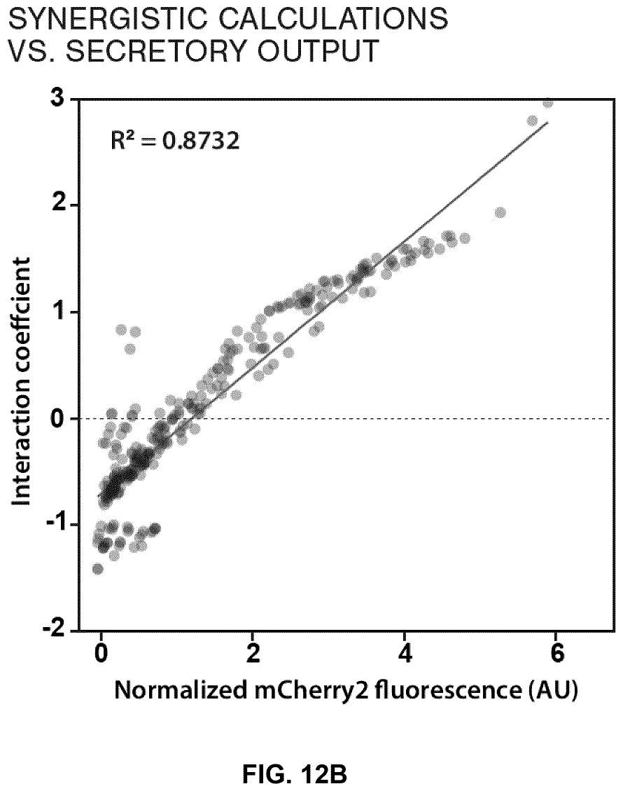

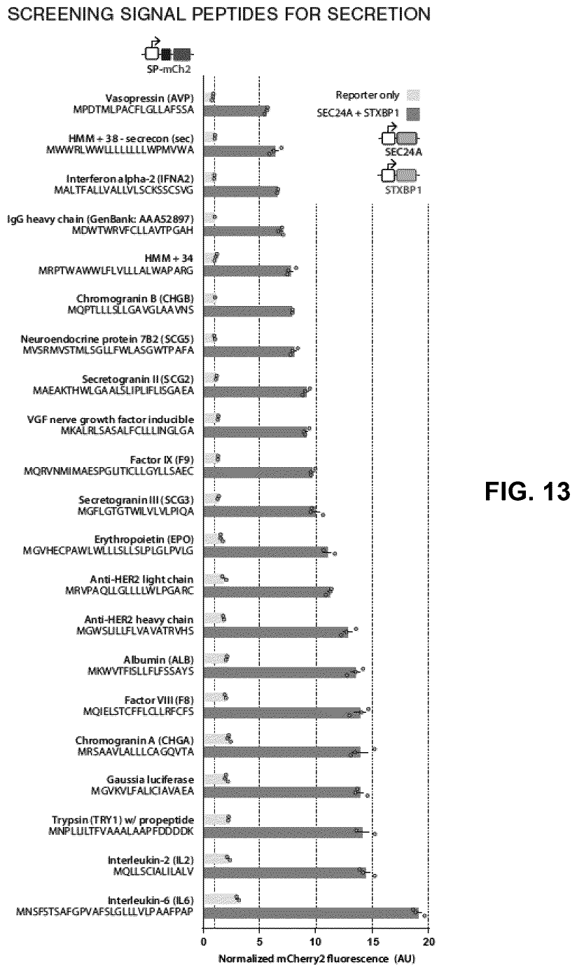

With STXBP1 showing the strongest synergy-promoting capacity within our initial screening, we conducted ternary factor transfection screening centered around STXBP1 to identify potential higher-order secretory synergies (FIG. 1C). Secretory factors selected for higher-order screening were chosen for their ability to synergize with STXBP1 (IC>0.25), which also selected for all secretion-boosting factors with “self-synergy” IC>0.1. Based on this screen's best three-factor secretory enhancements, we assembled multigene secretory programs consisting of combinations of the factors STXBP1, SEC24A, and SNAP23 (FIG. 1D, FIG. 12). Stoichiometrically controlled co-expression of these factors on a multigene expression cassette revealed that the synergistic augmentations made to transfected HEK cells' bioproduction capacity corresponded to increased intracellular content of secreted reporter, suggesting enhanced throughput enabled by overexpressed secretory machinery. Super-resolution fluorescence microscopy of transfected cells revealed that overexpressed secretory factors altered the distributions of secretory reporter across the endoplasmic reticulum, Golgi apparatus, plasma membrane, and cytosol (FIG. 1E).

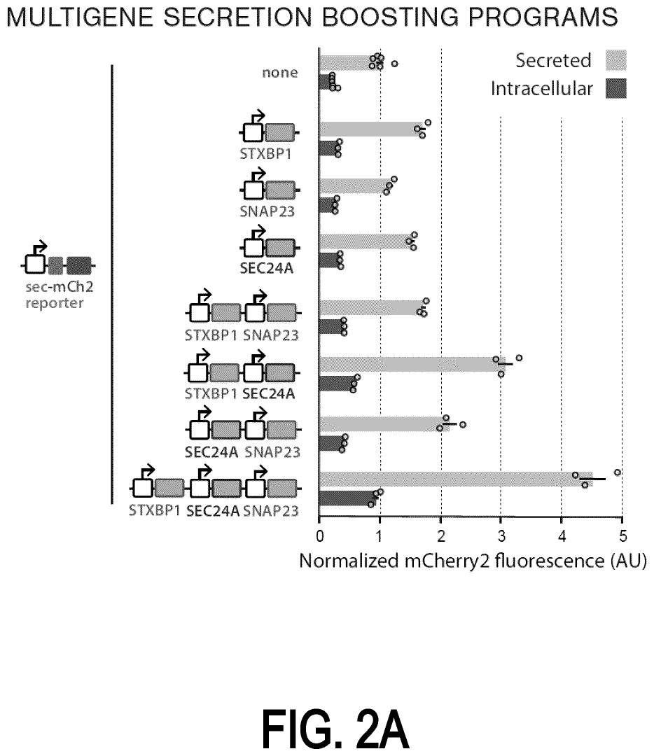

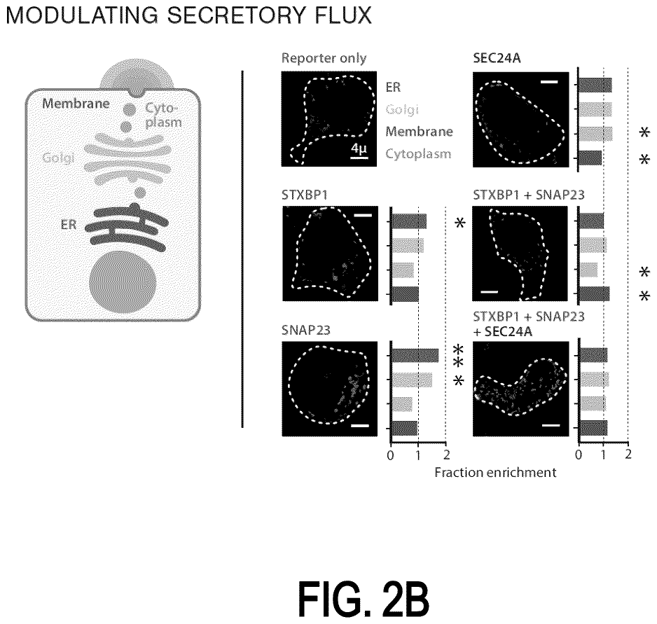

Using our strongest multigene secretory programs (e.g., STXBP1+SEC24A+SNAP23), we validated the ability of secretory factor overexpression to enhance bioproduction of therapeutically useful products, improve the secretory capacity of potential cell-based therapeutics, and establish synthetic control over bioproduction cell culture state. eGFP reporter-encoding lentivirus synthesized using transfected HEK293T cells exhibited greater potency when secretion-boosting secretory factors were co-transfected, improving efficiency when transducing HEK293T cells and increasing eGFP expression by up to 1.7× (FIG. 2A). MSCs transfected to secrete the anti-inflammatory cytokine IL-10 were augmented by co-transfection of our multigene secretory factor program, generating up to ??x greater secreted IL-10 output (FIG. 2B). HEK293T cells transfected to secrete anti-HER2 monoclonal antibody (mAb), EPO, or FA9 exhibited yield enhancements of ?.?x, ?.?x, and ?.?x, respectively using secretion-boosting secretory factors (FIG. 2C).

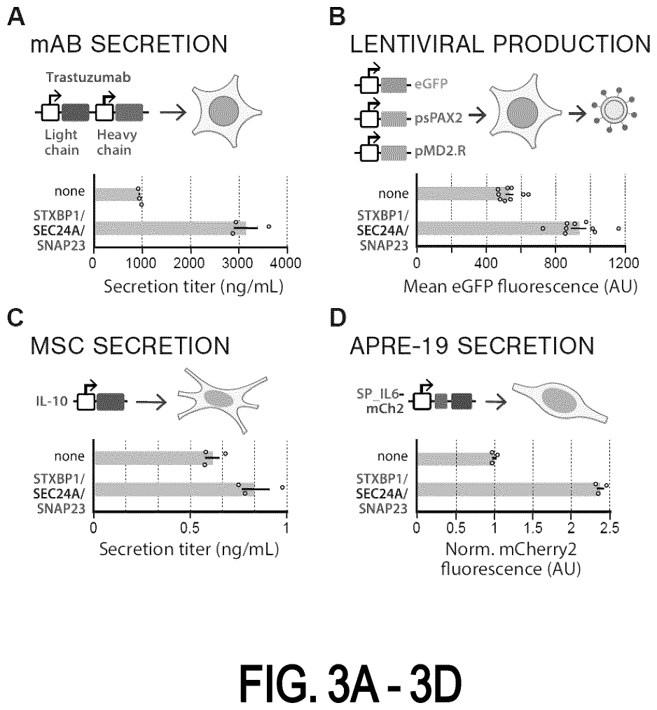

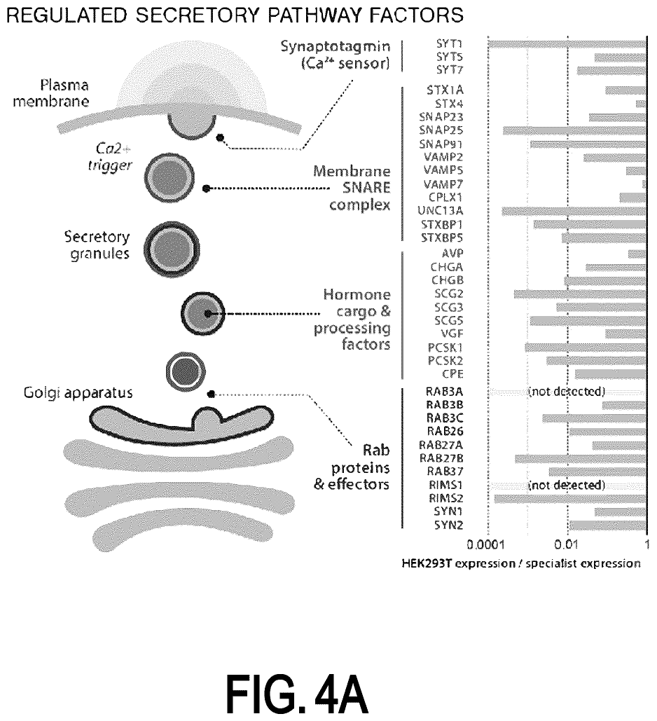

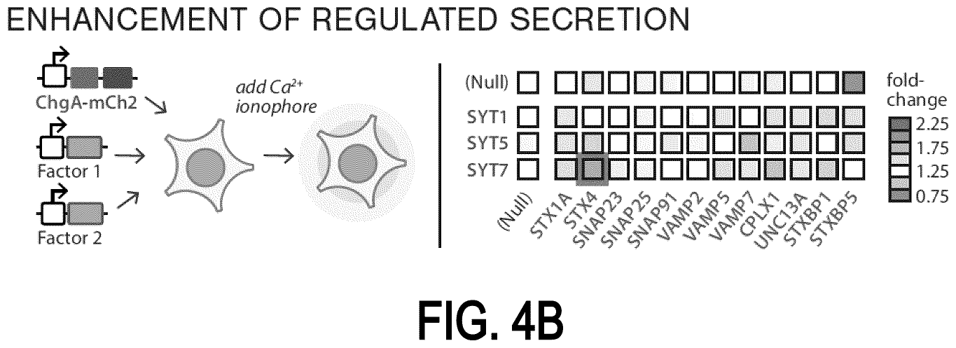

In contrast to the constitutive secretory processes active in all cells, regulated secretion in neuronal and neuroendocrine cells features specialized modifications of the secretory pathway, redirecting specific secretory cargos into dense-core granules comprise intracellular stockpiles of regulated secretory products (FIG. 3A). Upon activation of synaptotagmin to cytosolic Ca²⁺ influx, typically initiated by cellular signaling that activates membrane Ca²⁺ channels, these granules undergo exocytosis via membrane SNARE protein interactions gated by Ca²-synaptotagmin binding. We derived an archetypal transcriptomic profile for regulated secretory cells using bioinformatic analysis of 76 human primary cell lines downloaded from the Human Protein Atlas database, identifying professional regulated secretors by their expression levels of Ca²-sensing synaptotagmins (namely SYT1, SYT5, and SYT7) (Supplementary Table 5). Comparing the gene expression profile of this archetype to that of HEK293 cells revealed stark differences across a cross-section of known regulated secretory factors and cargo proteins. We leveraged this information to construct a transfection screen for reprogramming the secretory phenotype of HEK293T cells to enhance their capacity for regulated secretion.

Regulated secretory cargo proteins, such as the granin family of neurohormones, typically possess structural properties that help traffic them into regulated secretory granules as they mature along the secretory pathway. Ectopic expression of granins in canonically constitutive-only secretory cell lines such as COS fibroblasts can lead to formation of granule-like secretory bodies whose secretion rates increase in response to elevated cytosolic Ca²⁺. Based on this phenomenon, we constructed a regulated secretion reporter by fusing a C-terminal mCh2 domain to CHGA (FIG. 8). Microscopy imaging revealed that CHGA-mCh2 expressed in transfected HEK293T cells formed granules of substantially larger size than their constitutively secreting sec-mCh2 counterpart (FIG. 1E, FIG. 3E), although these granules were non-responsive to addition of the Ca²⁺ ionophore A23187.

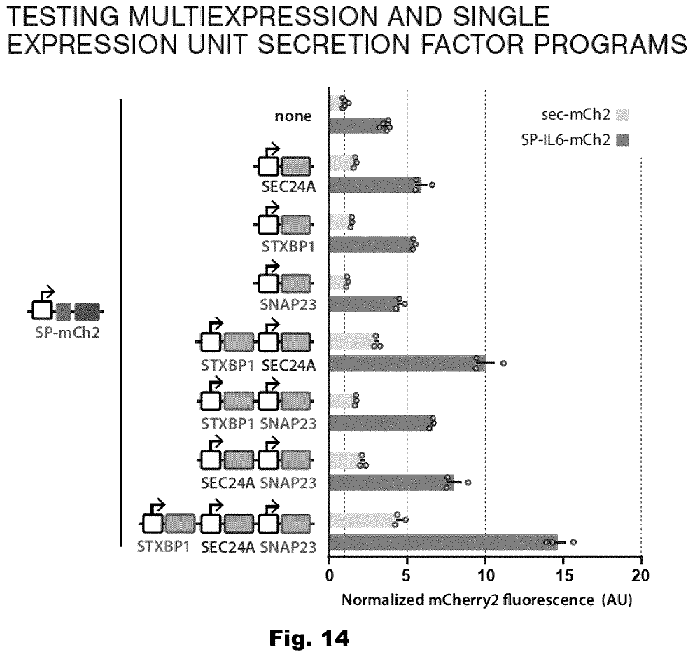

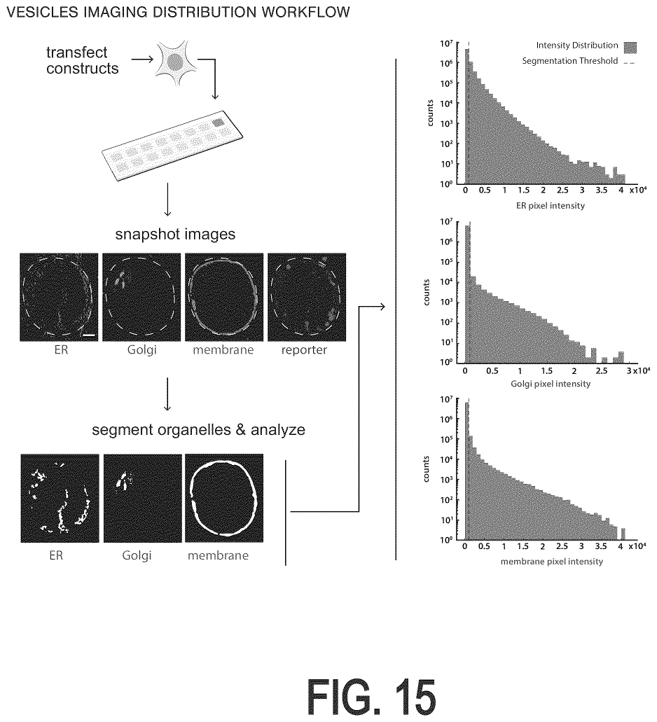

Because HEK293T cells transfected with CHGA-mCh2 failed to demonstrate substantially increased reporter secretion upon Ca²⁺ induction, we determined whether co-transfection of synaptotagmin and other secretory factors will constitute a synthetic Ca²⁺-inducible secretory response. Screening synaptotagmins against regulated secretory factors revealed that the combination of SYT7 and STX4 achieved a ~2× secretory induction response to 1 μM A23187 over the course of 3 h (FIG. 3B, FIG. 14), supporting proposed models of Ca²⁺-induced SYT-STX interactions as a regulated exocytosis trigger. Co-transfection of SYT7 and STX4 with additional regulated secretory factors could further enhance this regulated secretory induction by as high as ~3.5× (FIG. 3C). We consolidated several identified and potential secretory factor synergies into multigene expression cassettes for development and evaluation of regulated secretion programs (FIG. 3D, FIG. 15). Super-resolution microscopy of reporter granule activity in cells expressing these secretion programs reveals rapid ionophore-induced reporter secretion over the course of just 30 min (FIG. 3E). mCh2 imaging revealed subpopulations of Ca²⁺-responsive and non-responsive cells, with the former containing large secretory granules with ~4× the average diameter of smaller nonresponsive vesicles. The co-expression of SYT7 and STX4 enhanced formation of these large Ca²⁺-responsive granules, and additional co-expression of RAB26 and RAB27B further enhanced their formation.

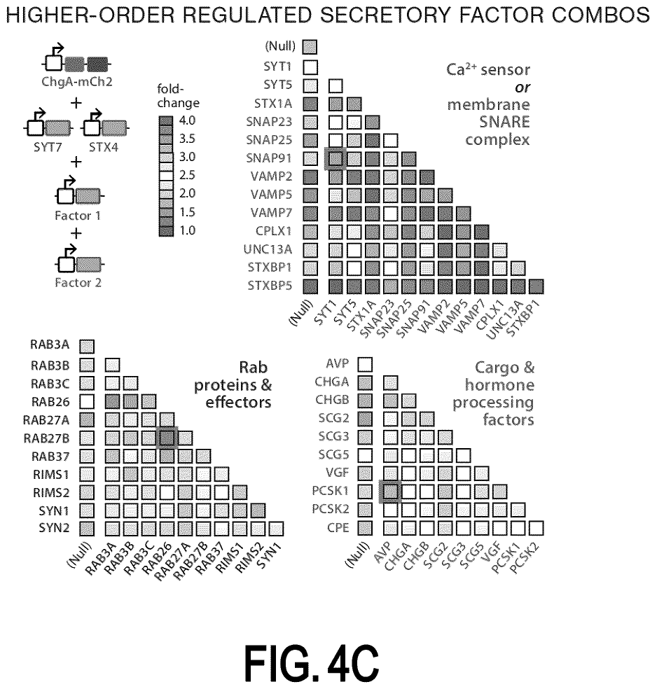

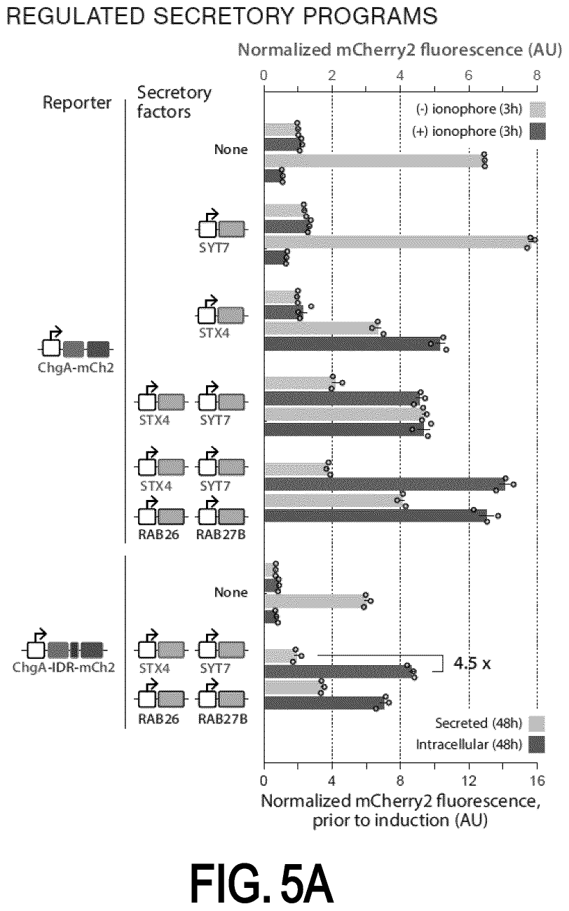

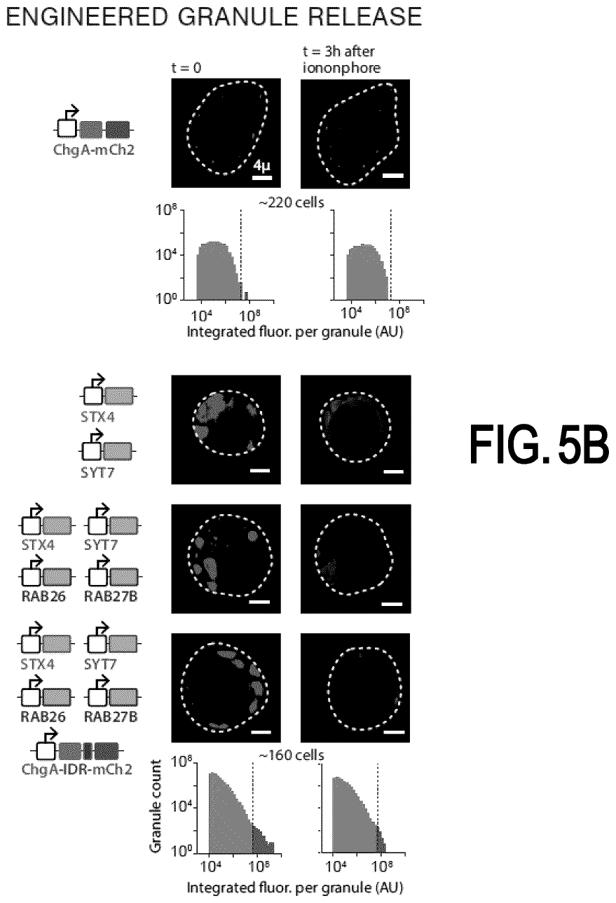

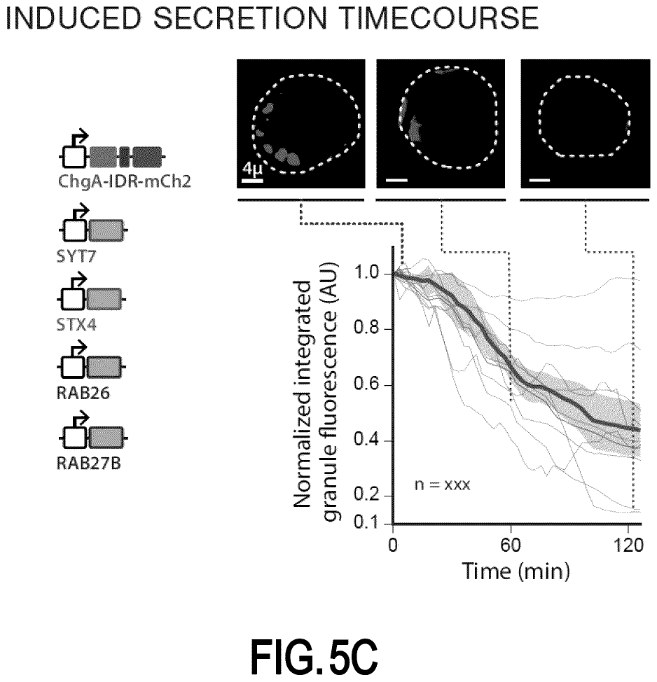

Imaging of regulated secretory granule exocytosis upon Ca²⁺ induction indicates that enhancement of large granule formation is critical for eliciting an engineered regulated secretory phenotype. This observation aligns with emerging models of cargo phase separation as a driver of regulated secretory cargo sorting and granule formation. However, most transfected cells show minimal formation of these granules, thus limiting the performance of our post-translational secretory induction circuits. For this reason, we substituted a synthetic intrinsically disordered region (IDR) as the flexible linker between the CHGA and the mCh2 reporter domains to facilitate phase separation of the cargo protein. After co-expressing our chosen regulated secretion factors with newly designed cargo protein, we found that including the IDR in the construct lowered basal secretory output compared to the design without it, though the dynamic range of secretory output decreased (FIG. 5A). With super resolution microscopy, we examined the dynamics of granule-based vesicle release by quantifying intracellular granule count and mCherry fluorescence of the cargo with and without the IDR linker and found more dramatic granule-based release from the IDR inclusive construct as well (15% decrease in fluorescence post-ionophore addition) (FIG. 5B). Granules seem to be depleted from the cell 3 hours post-ionophore addition, retaining ~30% of the initial mCherry signal (FIG. 5C).

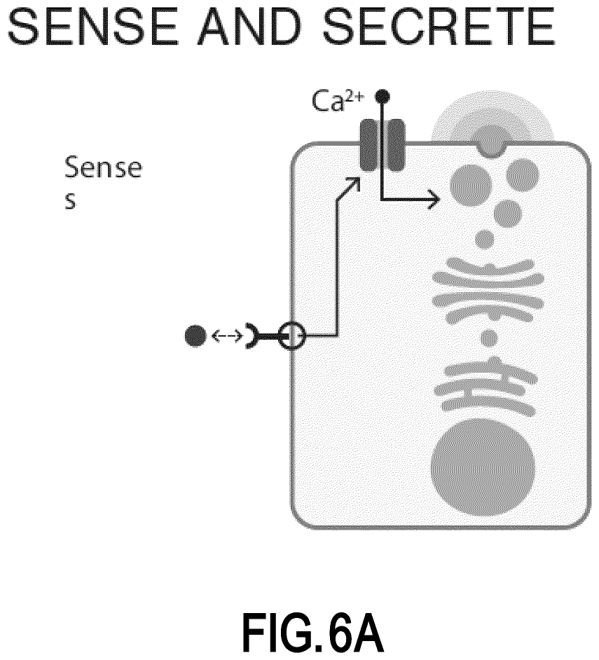

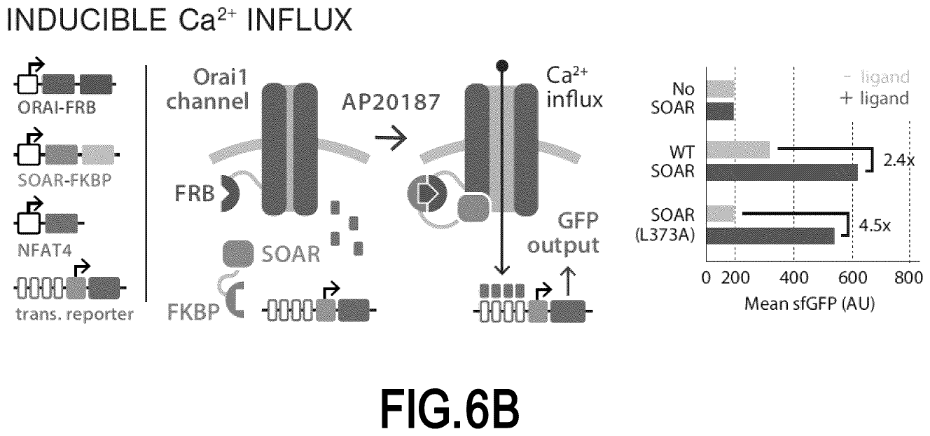

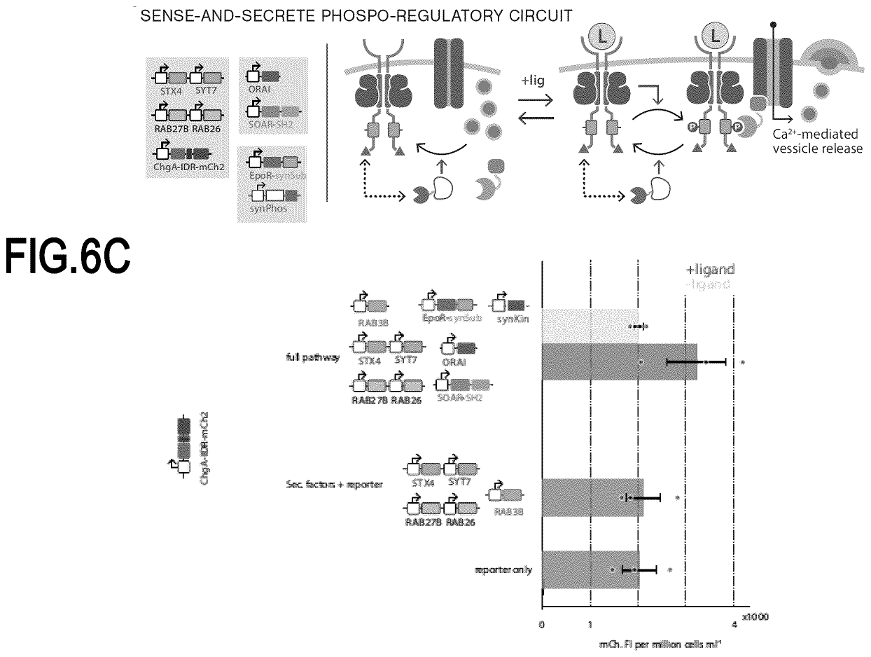

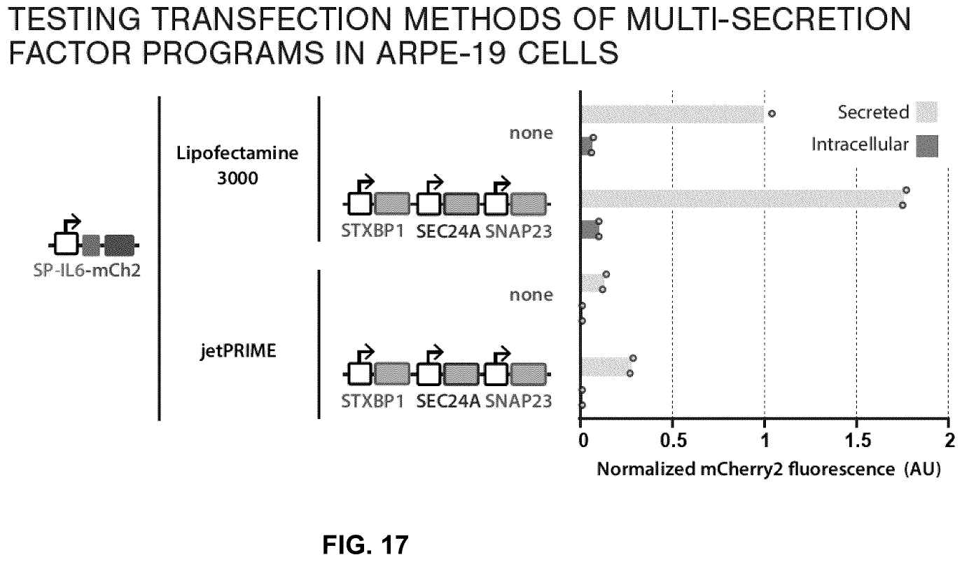

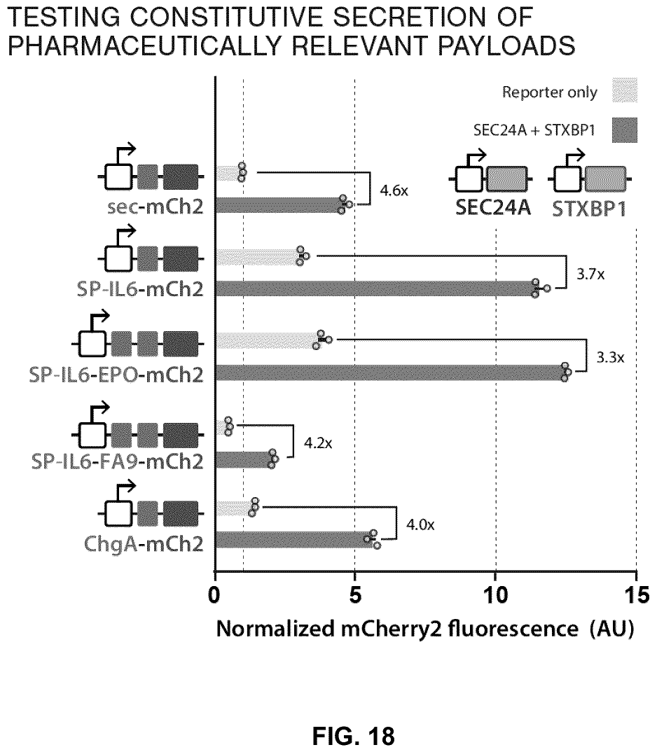

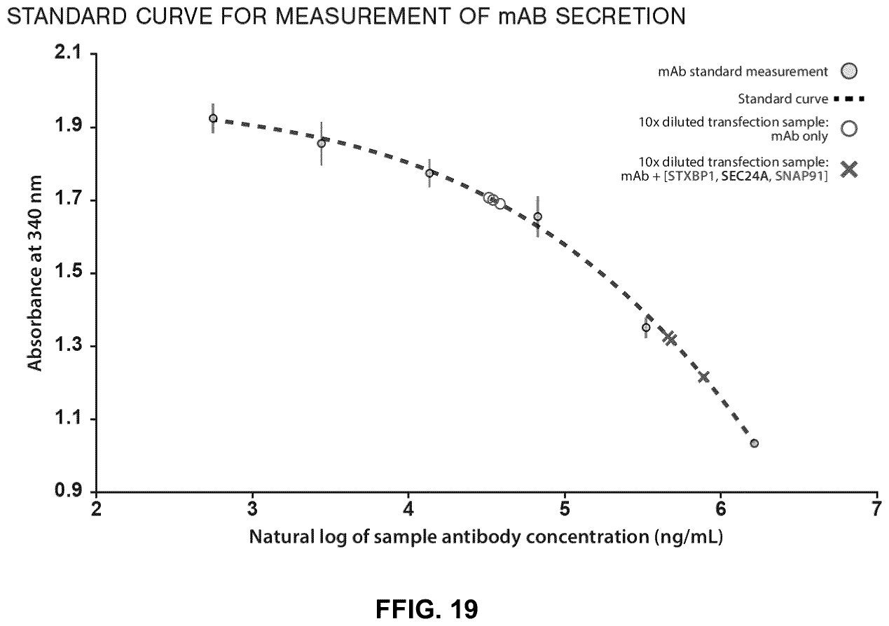

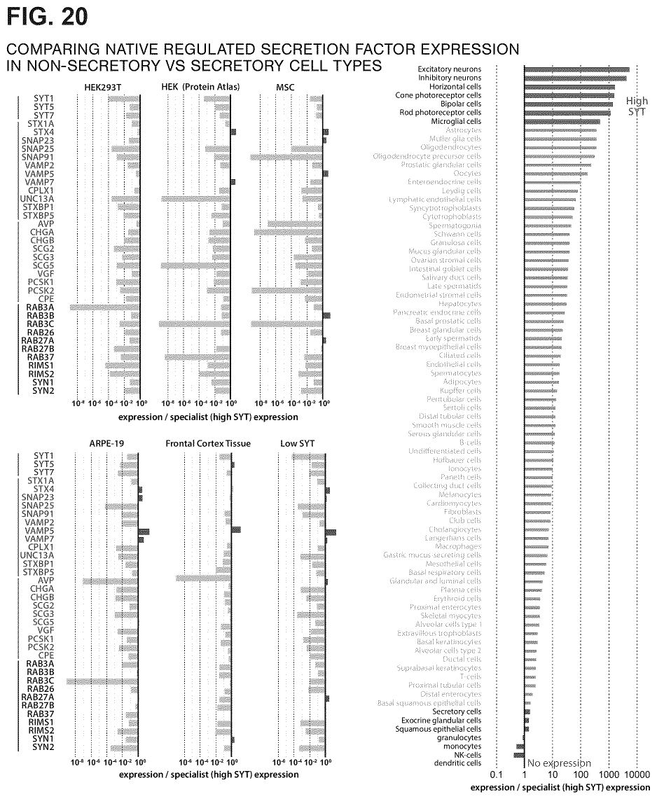

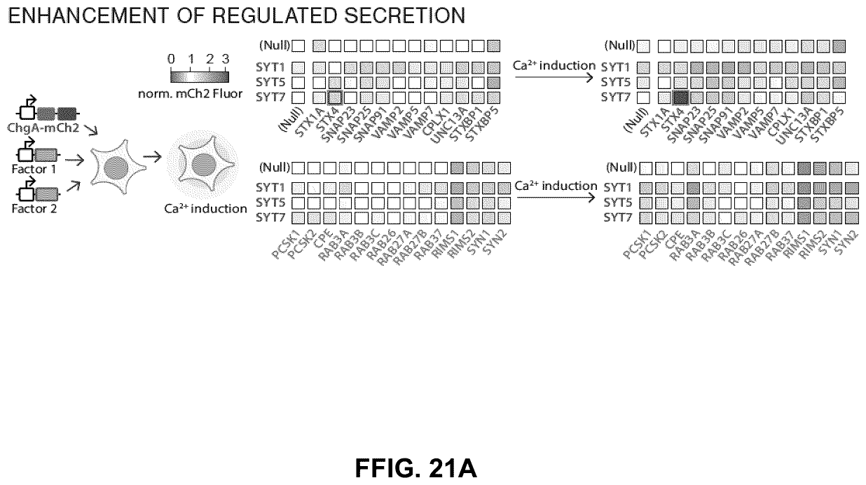

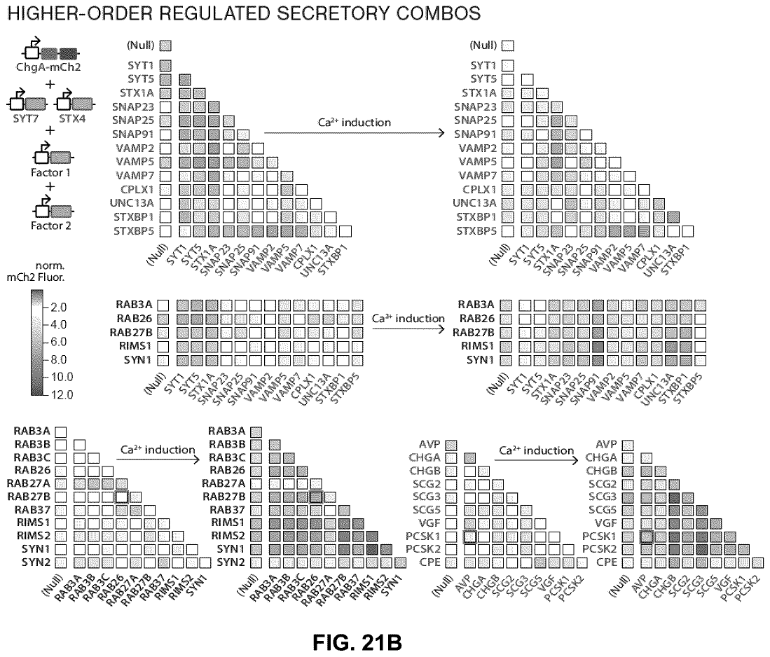

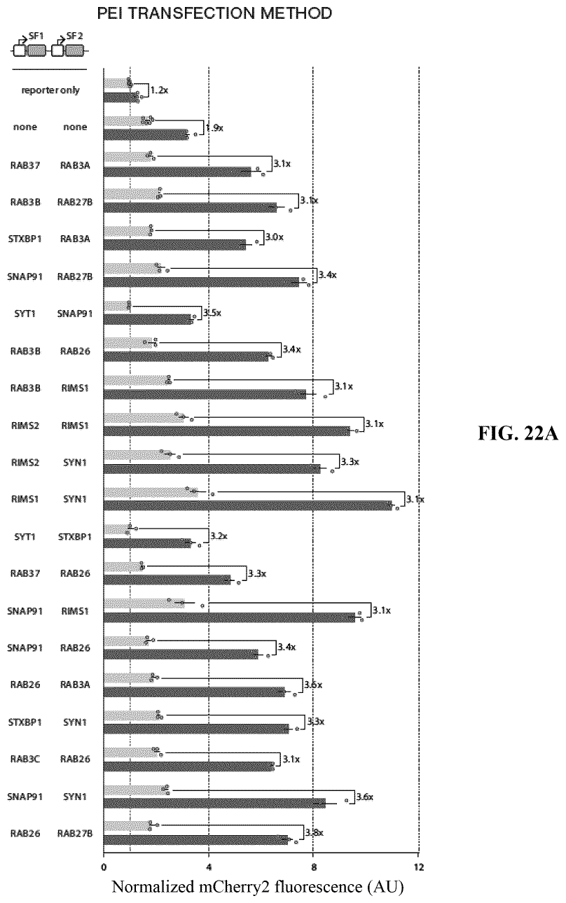

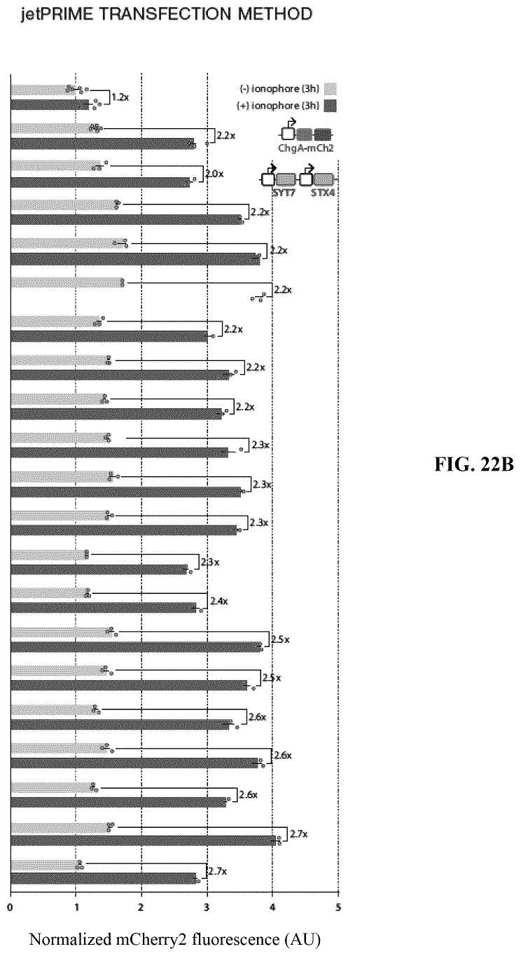

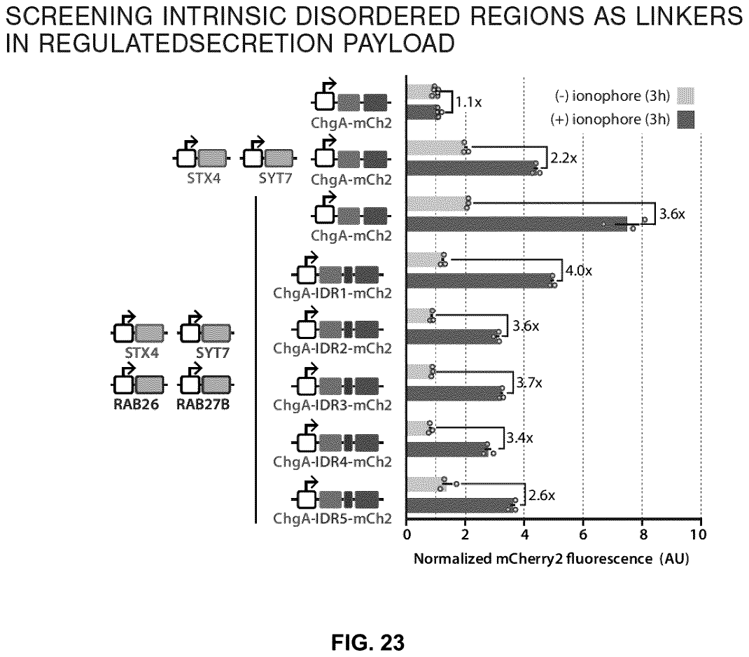

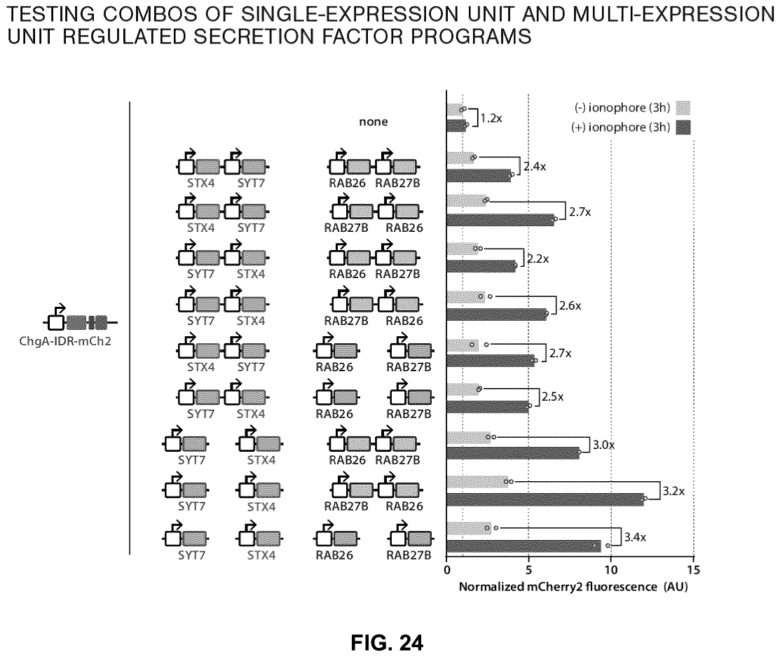

To implement a posttranslational sense-and-secrete circuit as an alternative to ligand-induced transcription, we needed to develop a synthetic ligand-gated Ca²⁺ channel circuit module to actuate our artificial regulated secretory system (FIG. 6A). We determined the ability of STIM1's SOAR domain to bind and activate Orai1, designing SOAR and Orai fusion constructs to construct rapalog-induced Ca²⁺ channel circuits (FIG. 17). To assess these circuits for Ca²⁺ channel activity in HEK293T cells, we developed a transcriptional cytosolic Ca²⁺ reporter by co-transfecting the Ca²⁺-activated transcription factor NFAT4 and a Ca²⁺-inducible sfGFP reporter with 9 repeats of the NFAT-binding promoter for IL8 (PNFAT), quantifying the transcriptional response to A23187 (ionophore carrier) via flow cytometry (FIG. 19). Using this reporter, we characterized Ca²⁺ influx following rapalog-induced recruitment of SOAR-FRB to Orai-FKBP, finding that wild-type SOAR generated significant constitutive activation of Orai (FIG. 6B, FIG. 20). By introducing point mutations to the coiled-coil region of SOAR responsible for binding to Orai's C-terminal tail, we attenuated basal SOAR-Orai activity and achieved up to 4.5× induction of sfGFP expression (using SOARL373A).

To build a synthetic membrane signaling pathway for controlling Ca²⁺ channel activation, we fused chosen mutant SOAR to a ZAP70 tandem SH2 adaptor domain, to serve as an effector protein to activate Orai1-mediated Ca²⁺ influx. We also engineered a synthetic receptor consisting of truncated EPOR fused to a synthetic substrate domain (CD3Z₃ₓ) and leucine zipper (FIG. 6C). Upon extracellular ligand binding, this membrane receptor and its cognate synKin would phosphorylate the fused synthetic substrate domain. The SOAR-SH2 effector protein would then localize to the phosphorylated substrate on the receptor via phosphotyrosine recognition on the synthetic substrate. enabling Orai1 channel activation from membrane proximity. Using the sfGFP reporter assay, we first quantified Ca²⁺ influx and cell health with flow cytometry.

We then plugged in our channel activation circuit to identified secretion modules to build a full synthetic sense-and-secrete pathway. We found that we had to readjust our transfection stoichi-ometries and co-transfect an additional secretory factor known as RAB3B to see more consistent activation. We found that our rapid, reversible sense-and-secrete pathway was able to show secretion of protein products within 30 minutes to an hour of ligand activation, reaching maximal, steady state levels at 2 hours, with a maximal induction of 1.61× of secretory output.

### DISCUSSION

As the global demands for these medicines increase, so must biopharmaceutical manufacturing capacity. We demonstrated and engineered a suite of post-translational modules to add secretory capability in human cell types that lack secretory function, and that we can use these programs to enhance function in cell types that have native secretory capability. Finally, we showed that we can engineer sense-and-secrete pathways with these programs that activate rapidly and reversibly via synthetic protein effectors that connect to Ca²⁺ channel machinery.

We first sought to endow synthetic constitutive secretion function to HEK293T cells, an immortalized cell line while frequently used in bioengineering contexts, lacked native secretory capability. To do this, we screened for genes identified to be active in constitutive secretory function, searching for genes that were upregulated in professional secretory cell types compared to non-secretory cell types with a systematic bioinformatic analysis. From this analysis, we selected certain genes and expressed them in combinations to identify which select constitutive programs could endow constitutive secretory function. We identified STXBP1 as one expression unit essential for constitutive secretion performance and proceeded with testing higher-order constitutive programs and quantifying secreted cargo localization with respect to these programs. We also demonstrated that our programs can be utilized to secrete diverse payloads, such as monoclonal antibodies and cytokines and that these programs can be expressed in other cell types to boost secretory phenotype. Our work in constitutive secretion engineering demonstrated how secretory phenotype could be augmented or entirely endowed by the expression of our synthetic constitutive programs, and furthermore, that our programs could be generalizable to diverse cell contexts and biomanufacturing applications.

Next, we wanted to engineer regulated secretory function; this is of great interest because being able to control secretion via chemical inputs or other effector mechanisms could be incredibly useful for precise cell therapeutics and controlling rates of bioproduction. We conducted a similar bioinformatic analysis as we did previously to identify genes, focusing on comparing designer secretory cell types that have native fine controlled secretion with cells lacking such function. We identified STX4 and SYT7 as regulated secretion factors capable of upregulating regulated machinery and screened them with higher-order combinations implicated in cargo and hormone processing. Additionally, we had to engineer our secretory payload further to facilitate cargo vesicle formation. We first fused CHGA as a domain to our payload and replaced our flexible linker domain with a linker containing an intrinsically disordered region. From our design, we found that certain IDR variations were able to better sequester secretory products pre-Ca²⁺ influx, reducing uncontrolled leak. With microscopy, we validated and showed that our synthetic regulated secretion programs facilitated the sequential export of our secretory products through intracellular machinery and compartments. Additionally, our cargo protein design is highly modular; our engineered vesicle formation domains are highly disordered and thus, can be easily fused to other recombinant proteins that may be of interest in biopharmaceuticals and therapeutics.

After we constructed these modules for regulated secretion, we then engineered a synthetic pathway for Ca²⁺ influx, to link small molecule inputs to Ca²⁺ channel activation. For cellular therapeutics, it is incredibly useful to sense small molecule disease markers rapidly and reversibly for and respond with therapeutic molecule secretion to ensure dynamic and specific treatment of a disease state. In the context of biologic production, tuning and toggling bioproduction rates with a small molecule can improve production efficiency, yield and quality without irreversibly compromising cell health in a batch culture.

Inspired by native neuroendocrine signaling pathways, we identified the minimal protein effectors for CRAC activation to be STIM1 (channel activator) and ORAI1 (membrane channel protein). We engineered STIM1 further by truncating it to its ORAI1 activating domain, SOAR, and introduced and screened numerous mutations in the SOAR domain to weaken non-specific, constitutive channel activation and improve inducibility. We then engineered a new synthetic receptor that triggers phosphorylation of a downstream synthetic substrate upon ligand binding. This receptor localizes to the membrane, and thus should be proximal to ORAI1 when expressed, so we used the phosphorylated receptor as a hub to drive recruitment of our engineered SOAR to the ORAI1 membrane channels upon ligand addition. To do this, we fused our engineered SOAR domain to a synthetic SH2 adaptor protein that binds to phosphotyrosine residues. Once we had demonstrated that ligand binding of our synthetic circuit could drive Ca²⁺ influx, we plugged these components in with our controlled secretion modules.

We found that we had to co-transfect additional RAB3B to see more consistent secretory inducibility with our full synthetic pathway. This may be because calcium influx due to ionophore (as we did in the regulated secretion factor screenings) is spatiotemporally distinct from Ca²⁺ driven by channel protein machinery. StimI-Orai1 machinery has been speculated to activate in highly localized multimer protein complexes at membrane junctions with differential levels of channel activity dependent on the multimer state, whereas ionophore addition is a uniform discharge of Ca²⁺ throughout the cell. The more localized and subtle nature of channel Ca²⁺ influxes may require additional factors such as RAB3B to increase secretory factor sensitivity; this factor is also markedly deficient in the HEK transcriptome.

We have previously shown that SH2 recruitment events to membrane proximal phosphotyrosine residues happen on a timescale of seconds to minutes; Ca²⁺ channel activation events, such as action potentials, happen rapidly on a timescale of milliseconds. This points out a key advantage of our circuits here—channel activation is not extremely rate limited by our receptor phosphorylation, minimizing the lag time between the ligand induction and secretory activity. Our circuits are also reversible, as we can inhibit substrate phosphorylation with small molecules to stop secretory function or co-transfect synthetic phosphatases that can toggle between ON/OFF phosphorylation states to adjust secretory rates as well. We showed substantial secretory output upon ligand activation within minutes, reaching maximal protein outputs in 2 hours. In stark contrast, previous work to build small-molecule induced secretion pathways have primarily relied on protein cleavage based systems that have 24-48 hour secretory timescales and are irreversible.

Our study extensively characterized post-translational modules to engineer human cells to gain or enhance existing secretory capability, offering insight into how native secretory machinery functions in human cells as well as defining suites of expression modules for engineering human cell bioproduction. Our engineered modules are payload-agnostic, enabling easy customization depending on context and application. Many clinically approved cell types such as MSCs, T cells, RPEs, already have existing endogenous secretory machinery, and would likely require only some of the components from the circuits and modules we have developed in this work, increasing ease of genetic modification. Finally, we engineered a calcium channel activation circuit by connecting a synthetic phosphorylation receptor to Orai1 membrane activation with extensive tuning and were able to plug these circuits into our identified controlled secretion modules. Our full synthetic sense-and-secrete pathway is a milestone demonstration in bottom-up engineering of the first rapid and reversible sense-and-secrete system published.

### Methods

### Plasmid Construction

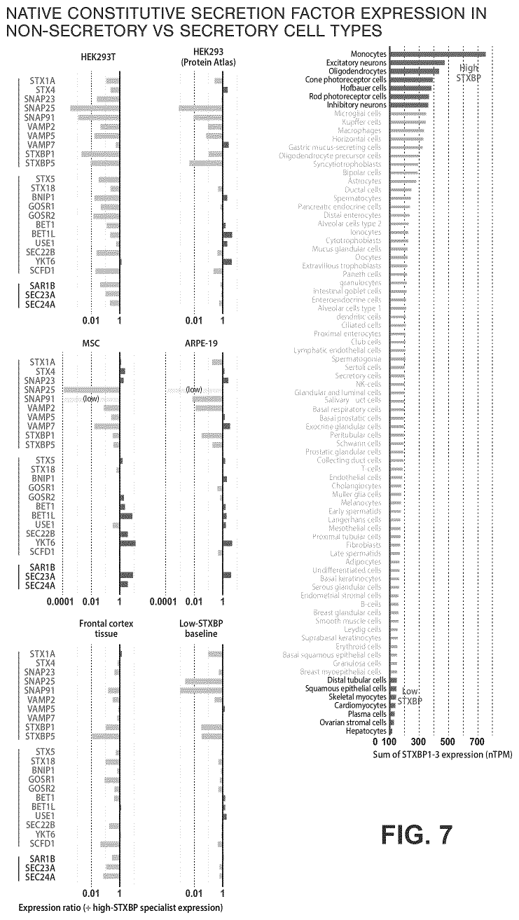

Plasmids used in this study are listed in Supplementary Table 1, and details of their experimental use are summarized in Supplementary Table 2. A custom hierarchical Golden Gate, Type II-S cloning scheme was used to construct the majority plasmids reported in this manuscript (FIG. 7). Input sequences for this scheme were commercially synthesized (Integrated DNA Technologies, Twist Bioscience) and PCR-amplified as needed. Some SOAR- and Orai1-containing constructs were cloned via standard Type II cloning methods where indicated (Supplementary Table 1). All plasmids were sequence-verified using Oxford Nanopore Technologies.

### HEK293T Cell Culture and Transfection

HEK293T cells (ATCC CRL®-11268™) were cultured in a humidity-controlled incubator at 37° C. with 5% CO₂ in Dulbecco's modified Eagle medium with high glucose (Gibco, 12100061) supplemented with 10% fetal bovine serum (FBS; GeminiBio, 900-108), 50 u/mL penicillin, 50 μg/mL streptomycin (Pen/Strep; Gibco, 15070063), and 2 mM 1-alanyl-l-glutamine (Caisson Labs, GLL02). ~1.5×10⁵ cells (<Passage 35) were plated in 24-well flat-bottom tissue culture plates (GenClone, 25-107) in 0.5 mL media, and they were transfected at 50-70% confluency following growth for ~36 h. For secretion plate reader quantification assays, plates were pre-treated using poly-l-lysine (PLL; R&D Systems, 343810001), and each transfection was performed in triplicate.

Cells were either transfected with polyethylenimine (PEI; 2.58 μL at 1 mg/mL for 1000 ng plasmid) or with jetPRIME (Polyplus, 101000046; 1 μL reagent and 50 μL per well for 500 ng plasmid) (see Supplementary Table 2). PEI stocks were prepared with linear PEI (Polysciences, 23966-2) dissolved at in Milli-Q® water, pH adjusted to 7.0 using NaOH and sterile-filtered (0.22 μm). Details of plasmid and transfection technique used in this study are detailed in Supplementary Table 2. 6 h after transfection, cell culture media was replaced with 0.5 mL of fresh complete DMEM. For secretion plate reader experiments, Gibco™ FluoroBrite™ DMEM (supplemented with FBS, Pen/Strep, and 1-alanyl-l-glutamine in the amounts previously listed) was used for this step.

### Viral Vector Synthesis and Transduction

To produce lentivirus, HEK293T cells (passage <30) were seeded in a 6-well media plate without antibiotics (10×10⁵ cells/well). After ~24 h (approximately 80% confluence), cells were transfected using jetPRIME. Media exchange was performed 4 h post-transfection. 24 h and 48 h following transfection, viral supernatant was harvested, filtered through a 0.45 μm PVDF filter, pooled, and stored at 4C prior to use.

For transduction, HEK293T cells were plated at a density of 150,000 cells/well in a 24-well plate. 24 h following plating (20-40% confluence), cells were transduced using viral supernatant with polybrene (MilliporeSigma) added to a final concentration of 8 μg/mL, and media was exchanged after 24 h incubation. 48 h following transduction, cells were harvested for analysis using flow cytometry. Precise details of plasmid and reagent usage for data used for this protocol are listed in Supplementary Table 2.

### MSC Nucleofection

MSC nucleofection was conducted using a Lonza 4D Nucleofector and the P1 Primary Cell 4D-Nucleofector X Kit (Lonza V4XP-1012). 3 million cells were thawed and resuspended in 100 μL of P1 primary cell buffer containing a total of 6 g of plasmid DNA (2 g DNA per million cells). The cell-DNA mixture was then moved to nucleofection cuvettes and electroporated using the Lonza 4D Nucleofector with the EW104 program code. After a 10 min recovery at 25° C., 500 L of pre-warmed complete α MEM was added to each cuvette. The cells were then split in three equal volumes and incubated in T25 cells culture flasks at 40,000 cells per cm² with 3 mL complete α-MEM.

### Secretion Assay

To quantify constitutive secretion from HEK293T cells (FIG. 1-3), media was exchanged at least 24 h after media transfection ensuring to use Fluorobrite DMEM with supplement, 100 μL of supernatant was transferred to solid black round-bottom 96-well plates (Corning™ 3792) at 100 μL/well (of 500 μL supernatant) and analyzed using Tecan Spark fluorescence/luminescence plate reader. Plate reader programmed settings are detailed in Supplementary Table 3. For intracellular reporter quantification, cells were collected using 200 μL TrypLE™ Express (Gibco, 12604021) resuspended with an addition of 300 μL FluoroBrite DMEM (no supplement), and centrifuged for 15 min. Since our measurement was in fluorescence, we fused our protein products with oxidation resistant fluorescent proteins.

The supernatant was aspirated and the cell pellets were frozen prior to resuspension in 50 μL RIPA lysis buffer (Thermo Scientific™ 89900T) mixed with 0.5 μL Halt™ protease inhibitor cocktail (Thermo Scientific™ 78429). Following 15 min. incubation on ice, the lysed cell mixtures were resuspended with an addition of 450 μL FluoroBrite DMEM (no supplement) and briefly vortexed to homogenize. Following 15 min. centrifugation, 100 μL supernatant (of 500 μL supernatant) was loaded into 96-well plates and analyzed using the plate reader. For regulated secretion quantification, culture media was exchanged for 500 μL of supplemented FluoroBrite DMEM 3 h prior to sample collection. Ionophore-induced secretion was performed using media spiked 1 μM A23187 (MilliporeSigma, C7522; prepared by dissolving A23187 powder into 500 μM in DMSO), and corresponding ionophore(-) media was spiked with 0.2% DMSO. Rapalog-induced secretion was performed using media spiked with 0.2 μM of the heterodimerizer AP21967 (Takara Bio USA, Inc., 635056).

### ELISA Assay for High-Value Pharmaceutical Products in MSCs

For ELISA analysis, 48 h following transfection, conditioned media samples from nucleofected MSCs were collected and frozen at −80° C. prior to ELISA analysis (BioLegend 430604) to quantify recombinant IL-10 secretory yields. Cells were harvested and counted using a Countess II and cell counting slides (Invitrogen C10283). The remaining cells were analyzed using flow cytometry to evaluate transfection efficiency.

### Staining Cellular Compartments

HEK293T cells were cultured in Dulbecco's Modified Eagle Medium (DMEM, Gibco) supplemented with 10% fetal bovine serum (FBS, Sigma-Aldrich) and 1% penicillin-streptomycin (Gibco) at 37° C. in a humidified atmosphere containing 5% CO₂. Cells were plated 24 hours before transfection at a density of 2×10⁵ cells per well in 24-well plates. Transfections were performed using JETPRIME reagent (Polyplus Transfection) according to the manufacturer's instructions. For imaging granules localizations, cells were co-transfected with a membrane-tagged iRFP construct, sec-mCherry (sec-mCH), and multi-SF Big Birds constructs at a 3:4:10 molar ratio. For imaging regulated secretory granules, 50 ng of CHGA-mCh2 or CHGA-IDR-mCh2 and 112.5 ng of each SF single expression units were co-transfected. After 24 hours, cells were trypsinized and replated into 8-well Ibidi chamber slides (Ibidi, 80806) at a density of 2×10⁴ cells per well for imaging.

To visualize the Golgi apparatus, CellLight Golgi-GFP BacMam 2.0 (Thermo Fisher Scientific, C10592) was added directly to the culture medium following the manufacturer's protocol. Briefly, the reagent was diluted to achieve a final concentration of 30 viral particles per cell. Cells were incubated for 24 hours at 37° C. in 5% CO₂ prior to imaging to allow expression of the fluorescent protein. The endoplasmic reticulum (ER) was stained using ER-Tracker™ Blue-White DPX dye (Thermo Fisher Scientific, E12353). A 1 μM working solution was prepared by diluting the stock solution in pre-warmed, serum-free medium. Cells were washed once with phosphate-buffered saline (PBS, pH 7.4) and incubated with the staining solution at 37° C. for 30 minutes, protected from light. Following incubation, cells were washed twice with PBS to remove excess dye and subsequently imaged. For imaging regulated secretory granules, ionophore was applied at the start of the imaging.

### Super Resolution Imaging

For Imaging, cells were maintained in pre-warmed, phenol red-free DMEM during imaging to minimize background fluorescence. Live-cell imaging was performed using structured illumination microscopy (SIM) on the Elyra 7 microscope (Carl Zeiss AG) equipped with Lattice SIM technology. Images were acquired using a 40× oil immersion objective lens (Plan Apochromat 40×/1.40 oil). Granules and dyes were excited with the following laser lines: 405 nm for ER-Tracker Blue-White DPX dye, 488 nm for CellLight Golgi-GFP, 561 nm for sec-mCherry, and 642 nm for membrane-tagged iRFP. The illumination pattern was configured to 5 phases, and z-stack acquisition was performed with a spacing of 0.5 m, optimized explicitly for “leap” acquisition protocols. Image reconstruction and processing were conducted using Zeiss ZEN 3.4 software with SIM2 algorithms. Time-lapse videos were recorded at 3-minute intervals over a duration of 3 hours in Lattice SIM mode.

### Live Cell Image Acquisition

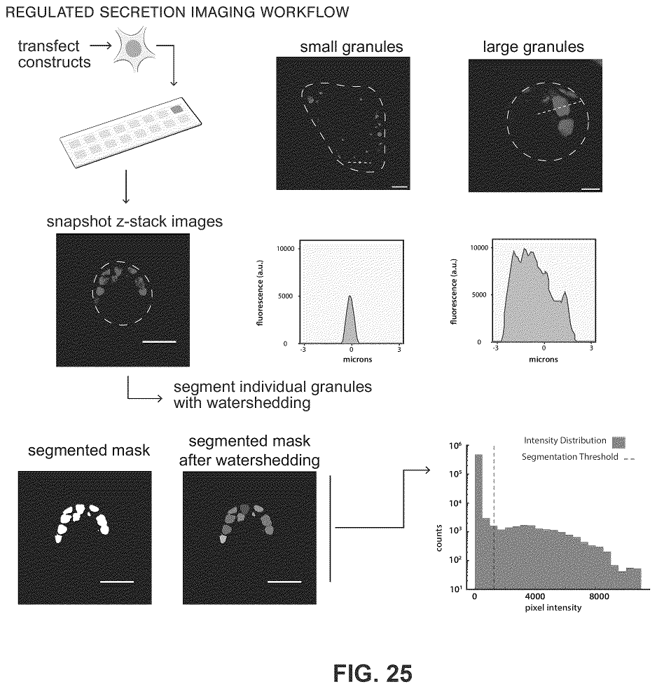

Imaging was conducted 48 h following transfection, with cells being replated onto Ibidi 8-chamber slides one day prior. Cells requiring addition of ionophore, rapalog, or blank media were treated without media replacement. Cellular imaging was conducted using a Zeiss Elyra 7 microscope with a Plan-Apochromat 40× objective lens (1.4 numerical aperture, oil immersion) and a PCO edge sCMOS camera. The acquisition of Apotome images was configured to capture five phase images at a camera exposure time of 30 milliseconds each. We employed 405, 488, 561, and 642 nm laser lines to excite ER-Tracker Blue-White DPX (ER), EmGFP (Golgi), mCh2 (reporter), and iRFP670 (plasma membrane), respectively. Optical z-sectioning of the cells was performed, utilizing a step size optimized explicitly for “leap” acquisition protocols. In the context of time-lapse imaging, z-stacks were acquired at 3-minute intervals. The Apotome phase images were processed using the Zeiss Zen Black software, applying the 3D SIM2 leap setting. For experiments examining regulated secretion, images from 20 fields of view (encompassing approximately 300 cells) were captured both before and after the addition of ionophore to ensure a comprehensive analysis. All dyes for staining organelles were applied in accordance with the guidelines provided by their respective manufacturers. Detailed microscope imaging settings are listed in Supplementary Table 4. For image analysis, we developed a custom MATLAB script and workflows for constitutive (FIG. 15) and regulated secretion (FIG. 25) pathway analysis.

### Flow Cytometry

For transfected cells, culture media was exchanged for complete DMEM without small molecule inducer or complete DMEM spiked with 0.2 μM AP21967 (Takara Bio) 36 h after transfection to initiate transcription of a fluorescent reporter gene (sfGFP). Transduced, transfected and nucleofected cells were all prepared for live-cell flow cytometry 48 h following initial transgene introduction.

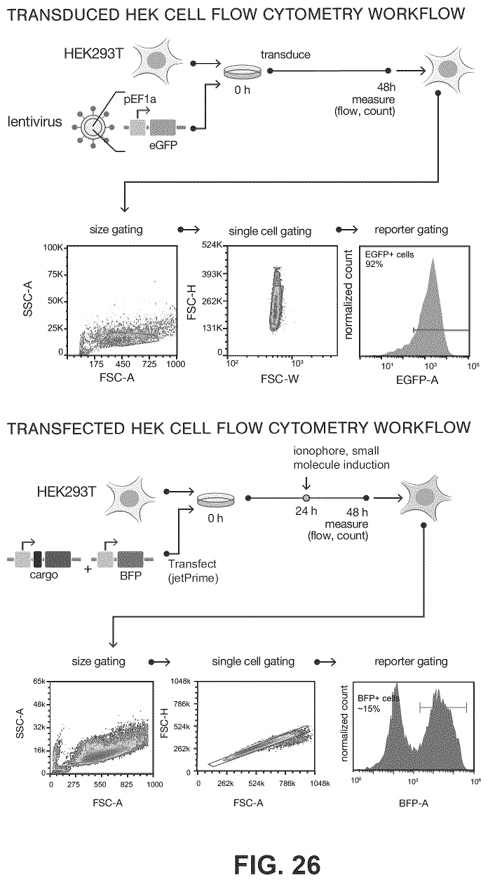

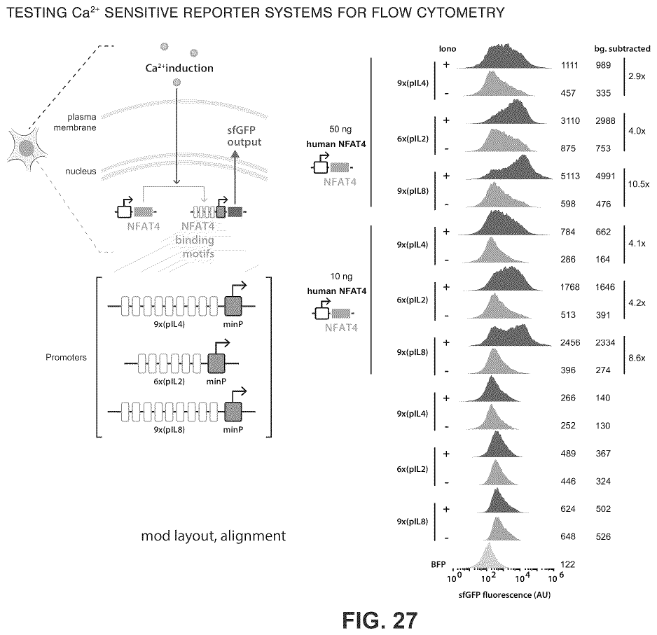

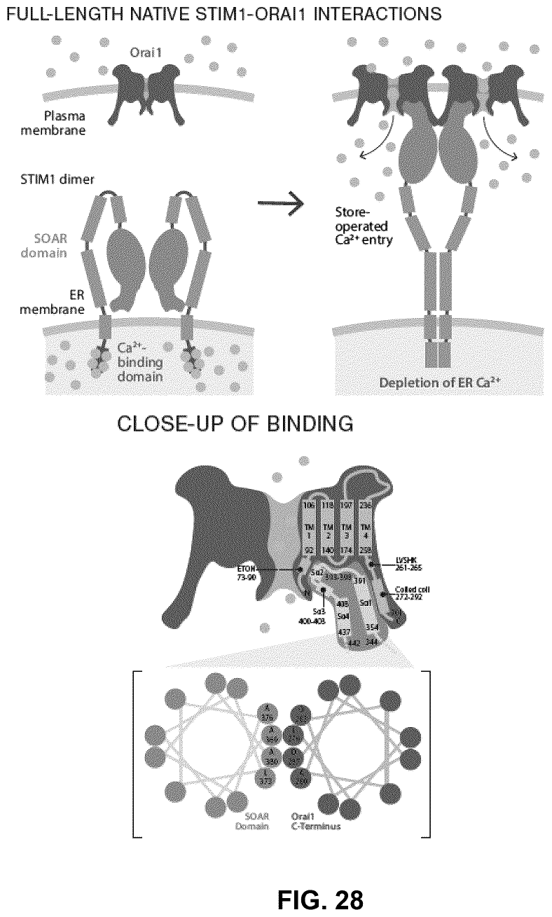

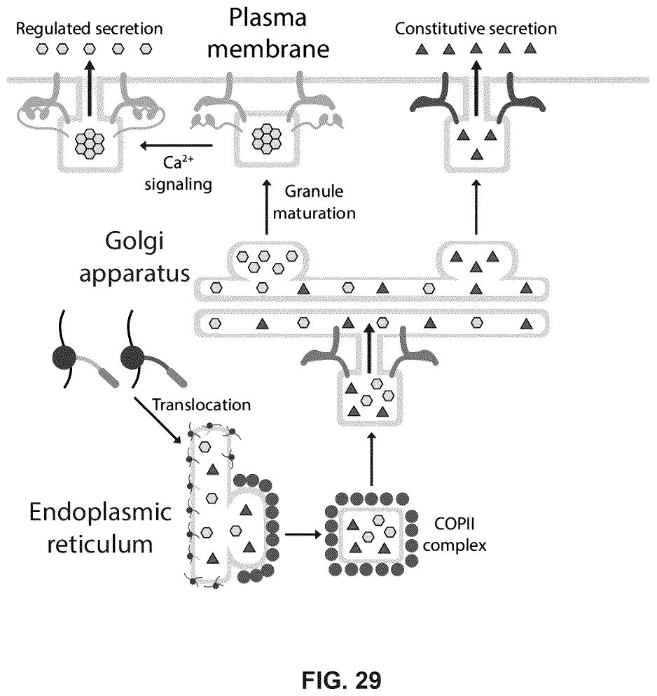

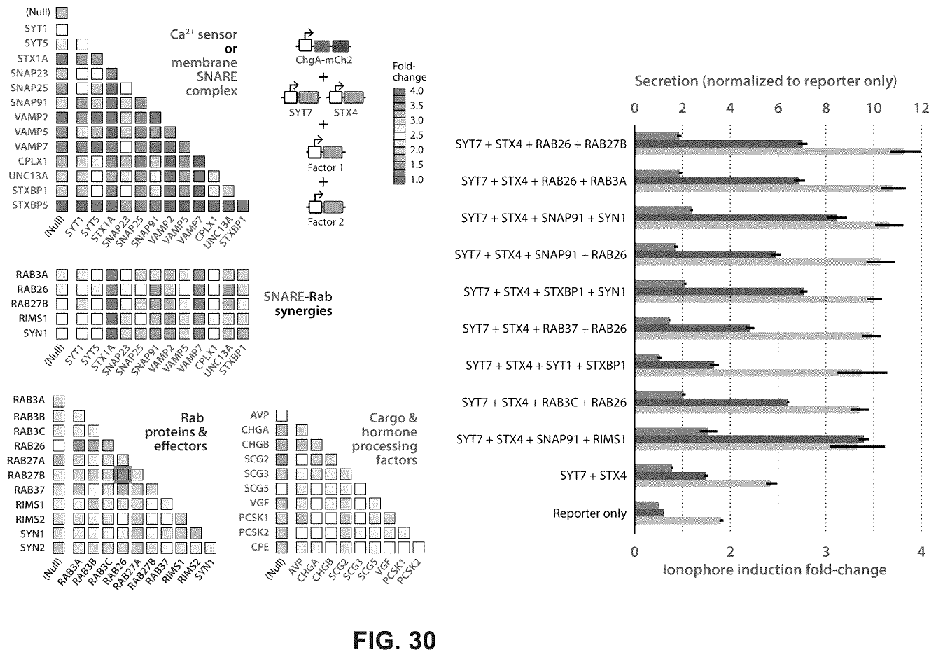

Prior to live-cell flow cytometry, media was aspirated, cells were washed with PBS, and TrypLE was added to detach cells. Samples were resuspended in complete DMEM and loaded into an SA3800 spectral cell analyzer (Sony Biotechnology) for analysis. ~1.5×10⁵ events were typically collected from each sample in a 24-well plate. Spectral unmixing of datasets was conducted using built-in software calibrated using single-color controls (cells transfected to express a single fluorescent protein). Details of experimental design, analysis methods, and analyzer settings are listed in Supplementary Table 2, FIG. 16 and FIG. 26.

### Bioinformatic Analysis

HEK293 T bulk RNA-Seq data from O'Connell and Rai et al. (cite CLASSIC manuscript here) was compared against single-cell type RNA-Seq data from the Human Protein Atlas. The nTPM counts of four professional secretory cell types (plasma cells, hepatocytes, B cells, and pancreatic endocrine cells) were averaged to generate a representative transcriptomic profile for professional constitutive secretors. Of the 76 cell types listed on the Human Protein Atlas with single-cell RNA-seq data available, the top 10^{th} percentile of expressors for Ca²⁺-responsive synaptotagmins SYT1, SYT5, and SYT7 (sum of nTPM counts) were selected and the nTPM counts averaged to generate a representative transcriptomic profile for professional regulated secretors. The nTPM ratios of HEK293 T to these representative professional secretor profiles were used to generate bioinformatics plots used in FIG. 1A and FIG. 3A. Literature outlining the core constitutive and regulated secretory pathways were employed to select genes for bioinformatic analysis and subsequent screening. Constitutive secretory pathway analysis included core genes involved in COPII complex formation, ER-Golgi vesicle trafficking, and secretory vesicle fusion with the plasma membrane. Regulated secretory pathway analysis focused on regulated secretory granule maturation, hormone cargos and processing thereof, and proteins involved in docking granules to the plasma membrane and initiating exocytosis upon sensing Ca²⁺. Software programs used to generate these analyses can be found at (code repository: GitHub) and (supplemental Excel datasheet of bioinformatics summary) (Supplementary Table 5).

### D. References

Peng et al. The vesicle-trafficking protein munc18b increases the secretory capacity of mammalian cells. Metabolic Engineering. 2010.
Peng et al. Differential effect of exocytic SNAREs on the production of recombinant proteins in mammalian cells. Biotechnology and Bioengineering. 2011.
Jazbec et al. Proteolytically activated CRAC effectors through designed intramolecular inhibition. ACS SynBio. 2022.
Yang et al. Engineering synthetic phosphorylation signaling networks in human cells. bioRxiv, under review at Science. 2023.
Beuret et al. Expression of regulated secretory proteins is sufficient to generate granule—like structures in constitutively secreting cells. Journal of Biological Chemistry. 2004.
Stettler et al. Determinants for chromogranin A sorting into the regulated secretory pathway are also sufficient to generate granule-like structures in non-endocrine cells. Biochemical Journal. 2009.
Montero-Hadj adj e et al. Chomogranin A promotes peptide hormone sorting into mobile granules in constitutively and regulated secreting cells. Journal of Biological Chemistry. 2009

### Example 7—Supplementary Tables

SUPPLEMENTARY TABLE 1
Plasmid Information Used in this Study

| | Position 1 (AB) | | | Position 2 (BC) | | | Position 3 (CD) | | |
|---|---|---|---|---|---|---|---|---|---|
| | | | Termin- | | | Termin- | | | Termin- |
| Plasmid# | Promoter | ORF | ator | Promoter | ORF | ator | Promoter | ORF | ator |
| ANTI-HER2 | | | | | | | | | |
| Heavy | | | | | | | | | |
| ANTI-HER2 | | | | | | | | | |
| Light | | | | | | | | | |
| HMM + 34 | | | | | | | | | |
| HMM + 38-sec | | | | | | | | | |
| FIG25_ | | | | | | | | | |
| REPORTER | | | | | | | | | |
| pJHE064 | D1-CMV- | CPLX1 | bGH225 | | | | | | |
| | u100 | | | | | | | | |
| pJHE065 | D1-CMV- | SYT7 | bGH225 | | | | | | |
| | u100 | | | | | | | | |
| pJHE066 | D1-CMV- | VAMP7 | bGH225 | | | | | | |
| | u100 | | | | | | | | |
| pJHE067 | D1-CMV- | STX4 | bGH225 | | | | | | |
| | u100 | | | | | | | | |
| pJHE068 | D1-CMV- | SNAP23 | bGH225 | | | | | | |
| | u100 | | | | | | | | |
| pJHE069 | D1-CMV- | SYT1 | bGH225 | | | | | | |
| | u100 | | | | | | | | |
| pJHE070 | D1-CMV- | SNAP25 | bGH225 | | | | | | |
| | u100 | | | | | | | | |
| pJHE071 | D1-CMV- | STX1A | bGH225 | | | | | | |
| | u100 | | | | | | | | |
| pJHE072 | D1-CMV- | VAMP2 | bGH225 | | | | | | |
| | u100 | | | | | | | | |
| pJHE077 | D1-CMV- | VGF | bGH225 | | | | | | |
| | u100 | | | | | | | | |
| pJHE0109 | D1-CMV- | PCSK1 | bGH225 | | | | | | |
| | u100 | | | | | | | | |
| pJHE0110 | D1-CMV- | PCSK2 | bGH225 | | | | | | |
| | u100 | | | | | | | | |
| pJHE0111 | D1-CMV- | CPE | bGH225 | | | | | | |
| | u100 | | | | | | | | |
| pJHE0112 | D1-CMV- | RAB3A | bGH225 | | | | | | |
| | u100 | | | | | | | | |
| pJHE0113 | | (Myc)-(GST)- | | | | | | | |
| | | (GSx9)- | | | | | | | |
| | | (ExtSOAR_Mut_A3 | | | | | | | |
| | | 69V-ZipRR_A) | | | | | | | |
| pJHE0114 | | (Myc)-(GST)- | | | | | | | |
| | | (GSx9)- | | | | | | | |
| | | (ExtSOAR_Mut_A3 | | | | | | | |
| | | 69D-ZipRR_A) | | | | | | | |
| pJHE0115 | | (Myc)-(GST)- | | | | | | | |
| | | (GSx9)- | | | | | | | |
| | | (ExtSOAR_Mut_A3 | | | | | | | |
| | | 69K-ZipRR_A) | | | | | | | |
| pJHE0116 | | (Myc)-(GST)- | | | | | | | |
| | | (GSx9)- | | | | | | | |
| | | (ExtSOAR_Mut_A3 | | | | | | | |
| | | 69S-ZipRR_A) | | | | | | | |
| pJHE0117 | | (Myc)-(GST)- | | | | | | | |
| | | (GSx9)- | | | | | | | |
| | | (ExtSOAR_Mut_A3 | | | | | | | |
| | | 69G-ZipRR_A) | | | | | | | |
| pJHE0118 | | (Myc)-(GST)- | | | | | | | |
| | | (GSx9)- | | | | | | | |
| | | (ExtSOAR_Mut_L3 | | | | | | | |
| | | 73A-ZipRR_A) | | | | | | | |
| pJHE0119 | | (Myc)-(GST)- | | | | | | | |
| | | (GSx9)- | | | | | | | |
| | | (ExtSOAR_Mut_L3 | | | | | | | |
| | | 73V-ZipRR_A) | | | | | | | |
| pJHE0120 | | (Myc)-(GST)- | | | | | | | |
| | | (GSx9)- | | | | | | | |
| | | (ExtSOAR_Mut L3 | | | | | | | |
| | | 73S-ZipRR_A) | | | | | | | |
| pJHE0121 | | (Myc)-(GST)- | | | | | | | |
| | | (GSx9)- | | | | | | | |
| | | (ExtSOAR_Mut_A3 | | | | | | | |
| | | 76D-ZipRR_A) | | | | | | | |
| pJHE0122 | | (Myc)-(GST)- | | | | | | | |
| | | (GSx9)- | | | | | | | |
| | | (ExtSOAR_Mut_A3 | | | | | | | |
| | | 76K-ZipRR_A) | | | | | | | |
| pJHE0123 | | (Myc)-(GST)- | | | | | | | |
| | | (GSx9)- | | | | | | | |
| | | (ExtSOAR_Mut_A3 | | | | | | | |
| | | 76S-ZipRR_A) | | | | | | | |
| pJHE0124 | | (Myc)-(GST)- | | | | | | | |
| | | (GSx9)- | | | | | | | |
| | | (ExtSOAR_Mut_A3 | | | | | | | |
| | | 76G-ZipRR_A) | | | | | | | |
| pJHE0125 | | (Myc)-(GST)- | | | | | | | |
| | | (GSx9)- | | | | | | | |
| | | (ExtSOAR_Mut_A3 | | | | | | | |
| | | 80D-ZipRR_A) | | | | | | | |
| pJHE0126 | | (Myc)-(GST)- | | | | | | | |
| | | (GSx9)- | | | | | | | |
| | | (ExtSOAR_Mut_A3 | | | | | | | |
| | | 80K-ZipRR_A) | | | | | | | |
| pJHE0127 | | (Myc)-(GST)- | | | | | | | |
| | | (GSx9)- | | | | | | | |
| | | (ExtSOAR_Mut A3 | | | | | | | |
| | | 80S-ZipRR_A) | | | | | | | |
| pJHE0128 | | (Myc)-(GST)- | | | | | | | |
| | | (GSx9)- | | | | | | | |
| | | (ExtSOAR_Mut_A3 | | | | | | | |
| | | 80G-ZipRR_A) | | | | | | | |
| pJHE0130 | D1-CMV- | UNC13A | bGH225 | | | | | | |
| | u100 | | | | | | | | |
| pJHE0148 | D1-CMV- | RAB27A | bGH225 | | | | | | |
| | u100 | | | | | | | | |
| pJHE0150 | D1-CMV- | STXBP1 | bGH225 | | | | | | |
| | u100 | | | | | | | | |
| pJHE0159 | D1-CMV- | SEC24A | bGH225 | | | | | | |
| | u100 | | | | | | | | |
| pJHE0160 | D1-CMV- | SNAP91 | bGH225 | | | | | | |
| | u100 | | | | | | | | |
| pJHE0161 | D1-CMV- | STX5 | bGH225 | | | | | | |
| | u100 | | | | | | | | |
| pJHE0162 | D1-CMV- | STX18 | bGH225 | | | | | | |
| | u100 | | | | | | | | |
| pJHE0164 | D1-CMV- | RAB3B | bGH225 | | | | | | |
| | u100 | | | | | | | | |
| pJHE0165 | D1-CMV- | RAB26 | bGH225 | | | | | | |
| | u100 | | | | | | | | |
| pJHE0166 | D1-CMV- | RAB27B | bGH225 | | | | | | |
| | u100 | | | | | | | | |
| pJHE0168 | D1-CMV- | RIMS2 | bGH225 | | | | | | |
| | u100 | | | | | | | | |
| pJHE0169 | D1-CMV- | SCG3 | bGH225 | | | | | | |
| | u100 | | | | | | | | |
| pJHE0170 | D1-CMV- | SCG5 | bGH225 | | | | | | |
| | u100 | | | | | | | | |
| pJHE0171 | D1-CMV- | STXBP5 | bGH225 | | | | | | |
| | u100 | | | | | | | | |
| pJHE0172 | D1-CMV- | SYN1 | bGH225 | | | | | | |
| | u100 | | | | | | | | |
| pJHE0173 | D1-CMV- | SYN2 | bGH225 | | | | | | |
| | u100 | | | | | | | | |
| pJHE0184 | D1-CMV- | VAMP5 | bGH225 | | | | | | |
| | u100 | | | | | | | | |
| pJHE0185 | D1-CMV- | CHGA | bGH225 | | | | | | |
| | u100 | | | | | | | | |
| pJHE0186 | D1-CMV- | RAB3C | bGH225 | | | | | | |
| | u100 | | | | | | | | |
| pJHE0188 | D1-CMV- | AVP | bGH225 | | | | | | |
| | u100 | | | | | | | | |
| pJHE0189 | D1-CMV- | SCG2 | bGH225 | | | | | | |
| | u100 | | | | | | | | |
| pJHE0195 | D1-CMV- | RIMS1 | bGH225 | | | | | | |
| | u100 | | | | | | | | |
| pJHE0219 | D1-CMV- | mCherry2 | bGH225 | | | | | | |
| | u100 | | | | | | | | |
| pJHE0222 | D1-CMV- | SP_IL6-mCherry2 | bGH225 | | | | | | |
| | u100 | | | | | | | | |
| pJHE0223 | D1-CMV- | SP_ALB-mCherry2 | bGH225 | | | | | | |
| | u100 | | | | | | | | |
| pJHE0224 | D1-CMV- | SP_IL2_Putative- | bGH225 | | | | | | |
| | u100 | mCherry2 | | | | | | | |
| pJHE0225 | D1-CMV- | SP_IFNA2- | bGH225 | | | | | | |
| | u100 | mCherry2 | | | | | | | |
| pJHE0226 | D1-CMV- | SP_GAUS_LUC- | bGH225 | | | | | | |
| | u100 | mCherry2 | | | | | | | |
| pJHE0227 | D1-CMV- | SP_IGG_GEN- | bGH225 | | | | | | |
| | u100 | mCherry2 | | | | | | | |
| pJHE0228 | D1-CMV- | SEC-SP_mCherry2 | bGH225 | | | | | | |
| | u100 | | | | | | | | |
| pJHE0230 | D1-CMV- | SP_TRY1_Propep- | bGH225 | | | | | | |
| | u100 | tide-mCherry2 | | | | | | | |
| pJHE0231 | D1-CMV- | SP_AVP-mCherry2 | bGH225 | | | | | | |
| | u100 | | | | | | | | |
| pJHE0232 | D1-CMV- | SP_CHGA- | bGH225 | | | | | | |
| | u100 | mCherry2 | | | | | | | |
| pJHE0233 | D1-CMV- | SP_CHGB- | bGH225 | | | | | | |
| | u100 | mCherry2 | | | | | | | |
| pJHE0234 | D1-CMV- | SP_SCG2- | bGH225 | | | | | | |
| | u100 | mCherry2 | | | | | | | |
| pJHE0235 | D1-CMV- | SP_SCG3- | bGH225 | | | | | | |
| | u100 | mCherry2 | | | | | | | |
| pJHE0236 | D1-CMV- | SP_SCG5- | bGH225 | | | | | | |
| | u100 | mCherry2 | | | | | | | |
| pJHE0237 | D1-CMV- | SP_VGF-mCherry2 | bGH225 | | | | | | |
| | u100 | | | | | | | | |
| pJHE0238 | D1-CMV- | SP_FA8-mCherry2 | bGH225 | | | | | | |
| | u100 | | | | | | | | |
| pJHE0239 | D1-CMV- | SP_FA9-mCherry2 | bGH225 | | | | | | |
| | u100 | | | | | | | | |
| pJHE0267 | D1-CMV- | SP EPO- | bGH225 | | | | | | |
| | u100 | mCherry2 | | | | | | | |
| pJHE0280 | D1-CMV- | SP_IL6-EPO- | bGH225 | | | | | | |
| | u100 | mCherry2 | | | | | | | |
| pJHE0281 | D1-CMV- | SEC-SP_EPO- | bGH225 | | | | | | |
| | u100 | mCherry2 | | | | | | | |
| pJHE0289 | D1-CMV- | SP_IL6-FA9- | bGH225 | | | | | | |
| | u100 | mCherry2 | | | | | | | |
| pJHE0290 | D1-CMV- | SEC-SP_FA9- | bGH225 | | | | | | |
| | u100 | mCherry2 | | | | | | | |
| pJHE0405 | | 6x(pIL2)-minP | | | | | | | |
| pJHE0411 | D1-CMV- | CHGB | bGH225 | | | | | | |
| | u100 | | | | | | | | |
| pJHE0412 | D1-CMV- | RAB37 | bGH225 | | | | | | |
| | u100 | | | | | | | | |
| pJHE0413 | D1-CMV- | SYT5 | bGH225 | | | | | | |
| | u100 | | | | | | | | |
| pJHE0423 | | 9x(pIL4)-minP | | | | | | | |
| pJHE0459 | D1-CMV- | NFAT4 | bGH225 | | | | | | |
| | u100 | | | | | | | | |
| pJHE0460 | D1-CMV- | CHGA-IDR_1- | bGH225 | | | | | | |
| | u100 | mCherry2 | | | | | | | |
| pJHE0461 | D1-CMV- | CHGA-IDR_2- | bGH225 | | | | | | |
| | u100 | mCherry2 | | | | | | | |
| pJHE0462 | D1-CMV- | CHGA-IDR_3- | bGH225 | | | | | | |
| | u100 | mCherry2 | | | | | | | |
| pJHE0463 | D1-CMV- | CHGA-IDR_4- | bGH225 | | | | | | |
| | u100 | mCherry2 | | | | | | | |
| pJHE0464 | D1-CMV- | CHGA-IDR_5- | bGH225 | | | | | | |
| | u100 | mCherry2 | | | | | | | |
| pJHE0478, | | SOAR_WT | | | | | | | |
| 483, 488 | | | | | | | | | |
| pJHE04XX | | 9x(pIL8)-minP | | | | | | | |
| jBgB09 | D1-CMV- | STXBP1 | bGH225 | D1-CMV- | SNAP23 | bGH225 | | | |
| | u100 | | | u100 | | | | | |
| jBgB021 | D1-CMV- | STXBP1 | bGH225 | D1-CMV- | SEC24A | bGH225 | | | |
| | u100 | | | u100 | | | | | |
| jBgB036 | D1-CMV- | SYT1 | bGH225 | D1-CMV- | SNAP91 | bGH225 | | | |
| | u100 | | | u100 | | | | | |
| jBgB039 | D1-CMV- | SYT1 | bGH225 | D1-CMV- | STXBP1 | bGH225 | | | |
| | u100 | | | u100 | | | | | |
| jBgB046 | D1-CMV- | RAB26 | bGH225 | D1-CMV- | RAB3A | bGH225 | | | |
| | u100 | | | u100 | | | | | |
| jBgB047 | D1-CMV- | RAB26 | bGH225 | D1-CMV- | RAB27B | bGH225 | | | |
| | u100 | | | u100 | | | | | |
| jBgB0108 | D1-CMV- | STX4 | bGH225 | D1-CMV- | SYT7 | bGH225 | | | |
| | u100 | | | u100 | | | | | |
| jBgB0110 | D1-CMV- | SYT7 | bGH225 | D1-CMV- | STX4 | bGH225 | | | |
| | u100 | | | u100 | | | | | |
| jBgB0117 | D1-CMV- | SNAP91 | bGH225 | D1-CMV- | SYN1 | bGH225 | | | |
| | u100 | | | u100 | | | | | |
| jBgB0147 | D1-CMV- | SNAP91 | bGH225 | D1-CMV- | RAB26 | bGH225 | | | |
| | u100 | | | u100 | | | | | |
| jBgB0148 | D1-CMV- | SNAP91 | bGH225 | D1-CMV- | RAB27B | bGH225 | | | |
| | u100 | | | u100 | | | | | |
| jBgB0149 | D1-CMV- | STXBP1 | bGH225 | D1-CMV- | SYN1 | bGH225 | | | |
| | u100 | | | u100 | | | | | |
| jBgB0150 | D1-CMV- | RAB3B | bGH225 | D1-CMV- | RAB26 | bGH225 | | | |
| | u100 | | | u100 | | | | | |
| jBgB0151 | D1-CMV- | RAB37 | bGH225 | D1-CMV- | RAB26 | bGH225 | | | |
| | u100 | | | u100 | | | | | |
| jBgB0152 | D1-CMV- | RIMS2 | bGH225 | D1-CMV- | SYN1 | bGH225 | | | |
| | u100 | | | u100 | | | | | |
| jBgB0153 | D1-CMV- | RAB3C | bGH225 | D1-CMV- | RAB26 | bGH225 | | | |
| | u100 | | | u100 | | | | | |
| jBgB0154 | D1-CMV- | RAB37 | bGH225 | D1-CMV- | RAB3A | bGH225 | | | |
| | u100 | | | u100 | | | | | |
| jBgB0155 | D1-CMV- | RAB3B | bGH225 | D1-CMV- | RIMS1 | bGH225 | | | |
| | u100 | | | u100 | | | | | |
| jBgB0156 | D1-CMV- | SNAP91 | bGH225 | D1-CMV- | RIMS1 | bGH225 | | | |
| | u100 | | | u100 | | | | | |
| jBgB0157 | D1-CMV- | RAB3B | bGH225 | D1-CMV- | RAB2 | bGH225 | | | |
| | u100 | | | u100 | 7B | | | | |
| jBgB0158 | D1-CMV- | RIMS2 | bGH225 | D1-CMV- | RIMS1 | bGH225 | | | |
| | u100 | | | u100 | | | | | |
| jBgB0159 | D1-CMV- | RIMS1 | bGH225 | D1-CMV- | SYN1 | bGH225 | | | |
| | u100 | | | u100 | | | | | |
| jBgB0160 | D1-CMV- | STXBP1 | bGH225 | D1-CMV- | RAB3A | bGH225 | | | |
| | u100 | | | u100 | | | | | |
| jBgB0175 | D1-CMV- | SEC24A | bGH225 | D1-CMV- | SNAP23 | bGH225 | | | |
| | u100 | | | u100 | | | | | |
| jBgB0176 | D1-CMV- | STXBP1 | bGH225 | D1-CMV- | SEC24A | bGH225 | D1-CMV- | SNAP | bGH225 |
| | u100 | | | u100 | | | u100 | 23 | |
| jBgB0195 | D1-CMV- | RAB27B | bGH225 | D1-CMV- | RAB26 | bGH225 | | | |
| | u100 | | | u100 | | | | | |

SUPPLEMENTARY TABLE 2
Plasmid and transfection usage

| | Plasmid # | | |
|---|---|---|---|
| Condition | And Usage (ng) | | Reagent |
| Reporter | pJHE0219 | | PEI |
| Only | 100 vs 1000 | | |
| | pJHE0228 | | |
| | 100 vs 1000 | | |
| | pJHE0281 | | |
| | 100 vs 1000 | | |
| | pJHE0290 | | |
| | 100 vs 1000 | | |
| | pJHE0200 | | |
| | 100 vs 1000 | | |
| Reporter + | pJHE0228 | pJHE0150 | |
| SF | 100 | 900 | |
| | | pJHE0159 | |
| | | 900 | |
| | | pJHE071 | |
| | | 900 | |
| | | pJHE067 | |
| | | 900 | |
| | | pJHE0161 | |
| | | 900 | |
| | | pJHE0162 | |
| | | 900 | |
| | | pJHE0171 | |
| | | 900 | |
| Reporter | pJHE0231 | | PEI |
| Only | 100 | | |
| | HMM + 38-sec | | |
| | 100 | | |
| | pJHE0225 | | |
| | 100 | | |
| | pJHE0227 | | |
| | 100 | | |
| | HMM + 34 | | |
| | 100 | | |
| | pJHE0233 | | |
| | 100 | | |
| | pJHE0236 | | |
| | 100 | | |
| | pJHE0234 | | |
| | 100 | | |
| | pJHE0237 | | |
| | 100 | | |
| | pJHE0239 | | |
| | 100 | | |
| | pJHE0235 | | |
| | 100 | | |
| | pJHE0267 | | |
| | 100 | | |
| | ANTI-HER2 Light | | |
| | 100 | | |
| | ANTI-HER2 Heavy | | |
| | 100 | | |
| | pJHE0223 | | |
| | 100 | | |
| | pJHE0238 | | |
| | 100 | | |
| | pJHE0232 | | |
| | 100 | | |
| | pJHE0226 | | |
| | 100 | | |
| | pJHE0230 | | |
| | 100 | | |
| | pJHE0224 | | |
| | 100 | | |
| | pJHE0222 | | |
| | 100 | | |
| Reporter + | pJHE0159, pJHE0150 | pJHE0231 | |
| SF | 450, 450 | 100 | |
| | | HMM + 38-sec | |
| | | 100 | |
| | | pJHE0225 | |
| | | 100 | |
| | | pJHE0227 | |
| | | 100 | |
| | | HMM + 34 | |
| | | 100 | |
| | | pJHE0233 | |
| | | 100 | |
| | | pJHE0236 | |
| | | 100 | |
| | | pJHE0234 | |
| | | 100 | |
| | | pJHE0237 | |
| | | 100 | |
| | | pJHE0239 | |
| | | 100 | |
| | | pJHE0235 | |
| | | 100 | |
| | | pJHE0267 | |
| | | 100 | |
| | | ANTI-HER2 Light | |
| | | 100 | |
| | | ANTI-HER2 Heavy | |
| | | 100 | |
| | | pJHE0223 | |
| | | 100 | |
| | | pJHE0238 | |
| | | 100 | |
| | | pJHE0232 | |
| | | 100 | |
| | | pJHE0226 | |
| | | 100 | |
| | | pJHE0230 | |
| | | 100 | |
| | | pJHE0224 | |
| | | 100 | |
| | | pJHE0222 | |
| | | 100 | |
| sec-mCH2 | pJHE0228 | pJHE0159 | PEI |
| | 100 | 900 | |
| | | pJHE0150 | |
| | | 900 | |
| | | pJHE068 | |
| | | 900 | |
| | | jBgB021 | |
| | | 900 | |
| | | jBgB09 | |
| | | 900 | |
| | | jBgB0175 | |
| | | 900 | |
| | | jBgB0176 | |
| | | 900 | |
| SP_IL6- | pJHE0222 | pJHE0159 | |
| mCH2 | 100 | 900 | |
| | | pJHE0150 | |
| | | 900 | |
| | | pJHE068 | |
| | | 900 | |
| | | jBgB021 | |
| | | 900 | |
| | | jBgB09 | |
| | | 900 | |
| | | jBgB0175 | |
| | | 900 | |
| | | jBgB0176 | |
| | | 900 | |
| Reporter | pJHE0222 | | Lipofectamine |
| Only | 50 | | 3000 |
| Reporter + | pJHE0222 | jBgB0176 | |
| SF | 50 | 450 | |
| Reporter | pJHE0222 | | jetPRIME |
| Only | 50 | | |
| Reporter + | pJHE0222 | jBgB0176 | |
| SF | 50 | 450 | |
| Reporter | pJHE0228 | | PEI |
| Only | 100 | | |
| | pJHE0222 | | |
| | 100 | | |
| | pJHE0280 | | |
| | 100 | | |
| | pJHE0289 | | |
| | 100 | | |
| | pJHE0200 | | |
| | 100 | | |
| Reporter + | jBgB021 | pJHE0228 | |
| SF | 900 | 100 | |
| | | pJHE0222 | |
| | | 100 | |
| | | pJHE0280 | |
| | | 100 | |
| | | pJHE0289 | |
| | | 100 | |
| | | pJHE0200 | |
| | | 100 | |
| Screen 1 | pJHE0200 | pJHE071 | PEI |
| | 100 | 225 | |
| | | pJHE067 | |
| | | 225 | |
| | | pJHE068 | |
| | | 225 | |
| | | pJHE070 | |
| | | 225 | |
| | | pJHE0160 | |
| | | 225 | |
| | | pJHE072 | |
| | | 225 | |
| | | pJHE0184 | |
| | | 225 | |
| | | pJHE066 | |
| | | 225 | |
| | | pJHE064 | |
| | | 225 | |
| | | pJHE0130 | |
| | | 225 | |
| | | pJHE0150 | |
| | | 225 | |
| | | pJHE0171 | |
| | | 225 | |
| | pJHE0200, pJHE069 | pJHE071 | |
| | 100, 225 | 225 | |
| | | pJHE067 | |
| | | 225 | |
| | | pJHE068 | |
| | | 225 | |
| | | pJHE070 | |
| | | 225 | |
| | | pJHE0160 | |
| | | 225 | |
| | | pJHE072 | |
| | | 225 | |
| | | pJHE0184 | |
| | | 225 | |
| | | pJHE066 | |
| | | 225 | |
| | | pJHE064 | |
| | | 225 | |
| | | pJHE0130 | |
| | | 225 | |
| | | pJHE0150 | |
| | | 225 | |
| | | pJHE0171 | |
| | | 225 | |
| | pJHE0200, pJHE0413 | pJHE071 | |
| | 100, 225 | 225 | |
| | | pJHE067 | |
| | | 225 | |
| | | pJHE068 | |
| | | 225 | |
| | | pJHE070 | |
| | | 225 | |
| | | pJHE0160 | |
| | | 225 | |
| | | pJHE072 | |
| | | 225 | |
| | | pJHE0184 | |
| | | 225 | |
| | | pJHE066 | |
| | | 225 | |
| | | pJHE064 | |
| | | 225 | |
| | | pJHE0130 | |
| | | 225 | |
| | | pJHE0150 | |
| | | 225 | |
| | | pJHE0171 | |
| | | 225 | |
| | pJHE0200, pJHE065 | pJHE071 | |
| | 100, 225 | 225 | |
| | | pJHE067 | |
| | | 225 | |
| | | pJHE068 | |
| | | 225 | |
| | | pJHE070 | |
| | | 225 | |
| | | pJHE0160 | |
| | | 225 | |
| | | pJHE072 | |
| | | 225 | |
| | | pJHE0184 | |
| | | 225 | |
| | | pJHE066 | |
| | | 225 | |
| | | pJHE064 | |
| | | 225 | |
| | | pJHE0130 | |
| | | 225 | |
| | | pJHE0150 | |
| | | 225 | |
| | | pJHE0171 | |
| | | 225 | |
| Screen 2 | pJHE0200 | pJHE0109 | PEI |
| | 100 | 225 | |
| | | pJHE0110 | |
| | | 225 | |
| | | pJHE0111 | |
| | | 225 | |
| | | pJHE0112 | |
| | | 225 | |
| | | pJHE0164 | |
| | | 225 | |
| | | pJHE0186 | |
| | | 225 | |
| | | pJHE0165 | |
| | | 225 | |
| | | pJHE0148 | |
| | | 225 | |
| | | pJHE0166 | |
| | | 225 | |
| | | pJHE0412 | |
| | | 225 | |
| | | pJHE0195 | |
| | | 225 | |
| | | pJHE0168 | |
| | | 225 | |
| | | pJHE0172 | |
| | | 225 | |
| | | pJHE0173 | |
| | | 225 | |
| | pJHE0200, pJHE069 | pJHE0109 | |
| | 100, 225 | 225 | |
| | | pJHE0110 | |
| | | 225 | |
| | | pJHE0111 | |
| | | 225 | |
| | | pJHE0112 | |
| | | 225 | |
| | | pJHE0164 | |
| | | 225 | |
| | | pJHE0186 | |
| | | 225 | |
| | | pJHE0165 | |
| | | 225 | |
| | | pJHE0148 | |
| | | 225 | |
| | | pJHE0166 | |
| | | 225 | |
| | | pJHE0412 | |
| | | 225 | |
| | | pJHE0195 | |
| | | 225 | |
| | | pJHE0168 | |
| | | 225 | |
| | | pJHE0172 | |
| | | 225 | |
| | | pJHE0173 | |
| | | 225 | |
| | pJHE0200, pJHE0413 | pJHE0109 | |
| | 100, 225 | 225 | |
| | | pJHE0110 | |
| | | 225 | |
| | | pJHE0111 | |
| | | 225 | |
| | | pJHE0112 | |
| | | 225 | |
| | | pJHE0164 | |
| | | 225 | |
| | | pJHE0186 | |
| | | 225 | |
| | | pJHE0165 | |
| | | 225 | |
| | | pJHE0148 | |
| | | 225 | |
| | | pJHE0166 | |
| | | 225 | |
| | | pJHE0412 | |
| | | 225 | |
| | | pJHE0195 | |
| | | 225 | |
| | | pJHE0168 | |
| | | 225 | |
| | | pJHE0172 | |
| | | 225 | |
| | | pJHE0173 | |
| | | 225 | |
| | pJHE0200, pJHE065 | pJHE0109 | |
| | 100, 225 | 225 | |
| | | pJHE0110 | |
| | | 225 | |
| | | pJHE0111 | |
| | | 225 | |
| | | pJHE0112 | |
| | | 225 | |
| | | pJHE0164 | |
| | | 225 | |
| | | pJHE0186 | |
| | | 225 | |
| | | pJHE0165 | |
| | | 225 | |
| | | pJHE0148 | |
| | | 225 | |
| | | pJHE0166 | |
| | | 225 | |
| | | pJHE0412 | |
| | | 225 | |
| | | pJHE0195 | |
| | | 225 | |
| | | pJHE0168 | |
| | | 225 | |
| | | pJHE0172 | |
| | | 225 | |
| | | pJHE0173 | |
| | | 225 | |
| Screen 3 | pJHE0200, pJHE065, | | PEI |
| | pJHE067 | | |
| | 100, 225, 225 | | |
| | pJHE0200, pJHE065, | | |
| | pJHE067, pJHE069 | | |
| | 100, 225, 225, 225 | | |
| | pJHE0200, pJHE065, | pJHE069 | |
| | pJHE067, pJHE0413 | 225 | |
| | 100, 225, 225, 225 | | |
| | pJHE0200, pJHE065, | pJHE069 | |
| | pJHE067, pJHE0171 | 225 | |
| | 100, 225, 225, 225 | pJHE0413 | |
| | | 225 | |
| | pJHE0200, pJHE065, | pJHE069 | |
| | pJHE067, pJHE068 | 225 | |
| | 100, 225, 225, 225 | pJHE0413 | |
| | | 225 | |
| | | pJHE071 | |
| | | 225 | |
| | pJHE0200, pJHE065, | pJHE069 | |
| | pJHE067, pJHE070 | 225 | |
| | 100, 225, 225, 225 | pJHE0413 | |
| | | 225 | |
| | | pJHE071 | |
| | | 225 | |
| | | pJHE068 | |
| | | 225 | |
| | pJHE0200, pJHE065, | pJHE069 | |
| | pJHE067, pJHE0160 | 225 | |
| | 100, 225, 225, 225 | pJHE0413 | |
| | | 225 | |
| | | pJHE071 | |
| | | 225 | |
| | | pJHE068 | |
| | | 225 | |
| | | pJHE070 | |
| | | 225 | |
| | pJHE0200, pJHE065, | pJHE069 | |
| | pJHE067, pJHE072 | 225 | |
| | 100, 225, 225, 225 | pJHE0413 | |
| | | 225 | |
| | | pJHE071 | |
| | | 225 | |
| | | pJHE068 | |
| | | 225 | |
| | | pJHE070 | |
| | | 225 | |
| | | pJHE0160 | |
| | | 225 | |
| | pJHE0200, pJHE065, | pJHE069 | |
| | pJHE067, pJHE0184 | 225 | |
| | 100, 225, 225, 225 | pJHE0413 | |
| | | 225 | |
| | | pJHE071 | |
| | | 225 | |
| | | pJHE068 | |
| | | 225 | |
| | | pJHE070 | |
| | | 225 | |
| | | pJHE0160 | |
| | | 225 | |
| | | pJHE072 | |
| | | 225 | |
| | pJHE0200, pJHE065, | pJHE069 | |
| | pJHE067, pJHE066 | 225 | |
| | 100, 225, 225, 225 | pJHE0413 | |
| | | 225 | |
| | | pJHE071 | |
| | | 225 | |
| | | pJHE068 | |
| | | 225 | |
| | | pJHE070 | |
| | | 225 | |
| | | pJHE0160 | |
| | | 225 | |
| | | pJHE072 | |
| | | 225 | |
| | | pJHE0184 | |
| | | 225 | |
| | pJHE0200, pJHE065, | pJHE069 | |
| | pJHE067, pJHE064 | 225 | |
| | 100, 225, 225, 225 | pJHE0413 | |
| | | 225 | |
| | | pJHE071 | |
| | | 225 | |
| | | pJHE068 | |
| | | 225 | |
| | | pJHE070 | |
| | | 225 | |
| | | pJHE0160 | |
| | | 225 | |
| | | pJHE072 | |
| | | 225 | |
| | | pJHE0184 | |
| | | 225 | |
| | | pJHE066 | |
| | | 225 | |
| | pJHE0200, pJHE065, | pJHE069 | |
| | pJHE067, pJHE0130 | 225 | |
| | 100, 225, 225, 225 | pJHE0413 | |
| | | 225 | |
| | | pJHE071 | |
| | | 225 | |
| | | pJHE068 | |
| | | 225 | |
| | | pJHE070 | |
| | | 225 | |
| | | pJHE0160 | |
| | | 225 | |
| | | pJHE072 | |
| | | 225 | |
| | | pJHE0184 | |
| | | 225 | |
| | | pJHE066 | |
| | | 225 | |
| | | pJHE064 | |
| | | 225 | |
| | pJHE0200, pJHE065, | pJHE069 | |
| | pJHE067, pJHE0150 | 225 | |
| | 100, 225, 225, 225 | pJHE0413 | |
| | | 225 | |
| | | pJHE071 | |
| | | 225 | |
| | | pJHE068 | |
| | | 225 | |
| | | pJHE070 | |
| | | 225 | |
| | | pJHE0160 | |
| | | 225 | |
| | | pJHE072 | |
| | | 225 | |
| | | pJHE0184 | |
| | | 225 | |
| | | pJHE066 | |
| | | 225 | |
| | | pJHE064 | |
| | | 225 | |
| | | pJHE0130 | |
| | | 225 | |
| | pJHE0200, pJHE065, | pJHE069 | |
| | pJHE067, pJHE0171 | 225 | |
| | 100, 225, 225, 225 | pJHE0413 | |
| | | 225 | |
| | | pJHE071 | |
| | | 225 | |
| | | pJHE068 | |
| | | 225 | |
| | | pJHE070 | |
| | | 225 | |
| | | pJHE0160 | |
| | | 225 | |
| | | pJHE072 | |
| | | 225 | |
| | | pJHE0184 | |
| | | 225 | |
| | | pJHE066 | |
| | | 225 | |
| | | pJHE064 | |
| | | 225 | |
| | | pJHE0130 | |
| | | 225 | |
| | | pJHE0150 | |
| | | 225 | |
| Screen 4 | pJHE0200, pJHE065, | pJHE069 | PEI |
| | pJHE067, pJHE0112 | 225 | |
| | | pJHE0413 | |
| | | 225 | |
| | | pJHE071 | |
| | | 225 | |
| | | pJHE068 | |
| | | 225 | |
| | | pJHE070 | |
| | | 225 | |
| | | pJHE0160 | |
| | | 225 | |
| | | pJHE072 | |
| | | 225 | |
| | | pJHE0184 | |
| | | 225 | |
| | | pJHE066 | |
| | | 225 | |
| | | pJHE064 | |
| | | 225 | |
| | | pJHE0130 | |
| | | 225 | |
| | | pJHE0150 | |
| | | 225 | |
| | | pJHE0171 | |
| | | 225 | |
| | pJHE0200, pJHE065, | pJHE069 | |
| | pJHE067, pJHE0165 | 225 | |
| | 100, 225, 225, 225 | pJHE0413 | |
| | | 225 | |
| | | pJHE071 | |
| | | 225 | |
| | | pJHE068 | |
| | | 225 | |
| | | pJHE070 | |
| | | 225 | |
| | | pJHE0160 | |
| | | 225 | |
| | | pJHE072 | |
| | | 225 | |
| | | pJHE0184 | |
| | | 225 | |
| | | pJHE066 | |
| | | 225 | |
| | | pJHE064 | |
| | | 225 | |
| | | pJHE0130 | |
| | | 225 | |
| | | pJHE0150 | |
| | | 225 | |
| | | pJHE0171 | |
| | | 225 | |
| | pJHE0200, pJHE065, | pJHE069 | |
| | pJHE067, pJHE0166 | 225 | |
| | 100, 225, 225, 225 | pJHE0413 | |
| | | 225 | |
| | | pJHE071 | |
| | | 225 | |
| | | pJHE068 | |
| | | 225 | |
| | | pJHE070 | |
| | | 225 | |
| | | pJHE0160 | |
| | | 225 | |
| | | pJHE072 | |
| | | 225 | |
| | | pJHE0184 | |
| | | 225 | |
| | | pJHE066 | |
| | | 225 | |
| | | pJHE064 | |
| | | 225 | |
| | | pJHE0130 | |
| | | 225 | |
| | | pJHE0150 | |
| | | 225 | |
| | | pJHE0171 | |
| | | 225 | |
| | pJHE0200, pJHE065, | pJHE069 | |
| | pJHE067, pJHE0195 | 225 | |
| | 100, 225, 225, 225 | pJHE0413 | |
| | | 225 | |
| | | pJHE071 | |
| | | 225 | |
| | | pJHE068 | |
| | | 225 | |
| | | pJHE070 | |
| | | 225 | |
| | | pJHE0160 | |
| | | 225 | |
| | | pJHE072 | |
| | | 225 | |
| | | pJHE0184 | |
| | | 225 | |
| | | pJHE066 | |
| | | 225 | |
| | | pJHE064 | |
| | | 225 | |
| | | pJHE0130 | |
| | | 225 | |
| | | pJHE0150 | |
| | | 225 | |
| | | pJHE0171 | |
| | | 225 | |
| | pJHE0200, pJHE065, | pJHE069 | |
| | pJHE067, pJHE0172 | 225 | |
| | 100, 225, 225, 225 | pJHE0413 | |
| | | 225 | |
| | | pJHE071 | |
| | | 225 | |
| | | pJHE068 | |
| | | 225 | |
| | | pJHE070 | |
| | | 225 | |
| | | pJHE0160 | |
| | | 225 | |
| | | pJHE072 | |
| | | 225 | |
| | | pJHE0184 | |
| | | 225 | |
| | | pJHE066 | |
| | | 225 | |
| | | pJHE064 | |
| | | 225 | |
| | | pJHE0130 | |
| | | 225 | |
| | | pJHE0150 | |
| | | 225 | |
| | | pJHE0171 | |
| | | 225 | |
| Screen 5 | pJHE0200, pJHE065, | | PEI |
| | pJHE067, pJHE0112 | | |
| | 100, 255, 255, 255 | | |
| | pJHE0200, pJHE065, | pJHE0112 | |
| | pJHE067, pJHE0164 | 225 | |
| | 100, 255, 255, 255 | | |
| | pJHE0200, pJHE065, | pJHE0112 | |
| | pJHE067, pJHE0186 | 225 | |
| | 100, 255, 255, 255 | pJHE0164 | |
| | | 225 | |
| | pJHE0200, pJHE065, | pJHE0112 | |
| | pJHE067, pJHE0165 | 225 | |
| | 100, 255, 255, 255 | pJHE0164 | |
| | | 225 | |
| | | pJHE0186 | |
| | | 225 | |
| | pJHE0200, pJHE065, | pJHE0112 | |
| | pJHE067, pJHE0148 | 225 | |
| | 100, 255, 255, 255 | pJHE0164 | |
| | | 225 | |
| | | pJHE0186 | |
| | | 225 | |
| | | pJHE0165 | |
| | | 225 | |
| | pJHE0200, pJHE065, | pJHE0112 | |
| | pJHE067, pJHE0166 | 225 | |
| | 100, 255, 255, 255 | pJHE0164 | |
| | | 225 | |
| | | pJHE0186 | |
| | | 225 | |
| | | pJHE0165 | |
| | | 225 | |
| | | pJHE0148 | |
| | | 225 | |
| | pJHE0200, pJHE065, | pJHE0112 | |
| | pJHE067, pJHE0412 | 225 | |
| | 100, 255, 255, 255 | pJHE0164 | |
| | | 225 | |
| | | pJHE0186 | |
| | | 225 | |
| | | pJHE0165 | |
| | | 225 | |
| | | pJHE0148 | |
| | | 225 | |
| | | pJHE0166 | |
| | | 225 | |
| | pJHE0200, pJHE065, | pJHE0112 | |
| | pJHE067, pJHE0195 | 225 | |
| | 100, 255, 255, 255 | pJHE0164 | |
| | | 225 | |
| | | pJHE0186 | |
| | | 225 | |
| | | pJHE0165 | |
| | | 225 | |
| | | pJHE0148 | |
| | | 225 | |
| | | pJHE0166 | |
| | | 225 | |
| | | pJHE0412 | |
| | | 225 | |
| | pJHE0200, pJHE065, | pJHE0112 | |
| | pJHE067, pJHE016 | 225 | |
| | 100, 255, 255, 255 | pJHE0164 | |
| | | 225 | |
| | | pJHE0186 | |
| | | 225 | |
| | | pJHE0165 | |
| | | 225 | |
| | | pJHE0148 | |
| | | 225 | |
| | | pJHE0166 | |
| | | 225 | |
| | | pJHE0412 | |
| | | 225 | |
| | | pJHE0195 | |
| | | 225 | |
| | pJHE0200, pJHE065, | pJHE0112 | |
| | pJHE067, pJHE0172 | 225 | |
| | 100, 255, 255, 255 | pJHE0164 | |
| | | 225 | |
| | | pJHE0186 | |
| | | 225 | |
| | | pJHE0165 | |
| | | 225 | |
| | | pJHE0148 | |
| | | 225 | |
| | | pJHE0166 | |
| | | 225 | |
| | | pJHE0412 | |
| | | 225 | |
| | | pJHE0195 | |
| | | 225 | |
| | | pJHE0168 | |
| | | 225 | |
| | pJHE0200, pJHE065, | pJHE0112 | |
| | pJHE067, pJHE0173 | 225 | |
| | 100, 255, 255, 255 | pJHE0164 | |
| | | 225 | |
| | | pJHE0186 | |
| | | 225 | |
| | | pJHE0165 | |
| | | 225 | |
| | | pJHE0148 | |
| | | 225 | |
| | | pJHE0166 | |
| | | 225 | |
| | | pJHE0412 | |
| | | 225 | |
| | | pJHE0195 | |
| | | 225 | |
| | | pJHE0168 | |
| | | 225 | |
| | | pJHE0172 | |
| | | 225 | |
| Screen 6 | pJHE0200, pJHE065, | | PEI |
| | pJHE067, pJHE0188 | | |
| | 100, 255, 255, 255 | | |
| | pJHE0200, pJHE065, | pJHE0188 | |
| | pJHE067, pJHE0185 | 225 | |
| | 100, 255, 255, 255 | | |
| | pJHE0200, pJHE065, | pJHE0188 | |
| | pJHE067, pJHE0411 | 225 | |
| | 100, 255, 255, 255 | pJHE0185 | |
| | | 225 | |
| | pJHE0200, pJHE065, | pJHE0188 | |
| | pJHE067, pJHE0189 | 225 | |
| | 100, 255, 255, 255 | pJHE0185 | |
| | | 225 | |
| | | pJHE0411 | |
| | | 225 | |
| | pJHE0200, pJHE065, | pJHE0188 | |
| | pJHE067, pJHE0169 | 225 | |
| | 100, 255, 255, 255 | pJHE0185 | |
| | | 225 | |
| | | pJHE0411 | |
| | | 225 | |
| | | pJHE0189 | |
| | | 225 | |
| | pJHE0200, pJHE065, | pJHE0188 | |
| | pJHE067, pJHE0170 | 225 | |
| | 100, 255, 255, 255 | pJHE0185 | |
| | | 225 | |
| | | pJHE0411 | |
| | | 225 | |
| | | pJHE0189 | |
| | | 225 | |
| | | pJHE0169 | |
| | | 225 | |
| | pJHE0200, pJHE065, | pJHE0188 | |
| | pJHE067, pJHE077 | 225 | |
| | 100, 255, 255, 255 | pJHE0185 | |
| | | 225 | |
| | | pJHE0411 | |
| | | 225 | |
| | | pJHE0189 | |
| | | 225 | |
| | | pJHE0169 | |
| | | 225 | |
| | | pJHE0170 | |
| | | 225 | |
| | pJHE0200, pJHE065, | pJHE0188 | |
| | pJHE067, pJHE0109 | 225 | |
| | 100, 255, 255, 255 | pJHE0185 | |
| | | 225 | |
| | | pJHE0411 | |
| | | 225 | |
| | | pJHE0189 | |
| | | 225 | |
| | | pJHE0169 | |
| | | 225 | |
| | | pJHE0170 | |
| | | 225 | |
| | | pJHE077 | |
| | | 225 | |
| | pJHE0200, pJHE065, | pJHE0188 | |
| | pJHE067, pJHE0110 | 225 | |
| | 100, 255, 255, 255 | pJHE0185 | |
| | | 225 | |
| | | pJHE0411 | |
| | | 225 | |
| | | pJHE0189 | |
| | | 225 | |
| | | pJHE0169 | |
| | | 225 | |
| | | pJHE0170 | |
| | | 225 | |
| | | pJHE077 | |
| | | 225 | |
| | | pJHE0109 | |
| | | 225 | |
| | pJHE0200, pJHE065, | pJHE0188 | |
| | pJHE067, pJHE0111 | 225 | |
| | 100, 255, 255, 255 | pJHE0185 | |
| | | 225 | |
| | | pJHE0411 | |
| | | 225 | |
| | | pJHE0189 | |
| | | 225 | |
| | | pJHE0169 | |
| | | 225 | |
| | | pJHE0170 | |
| | | 225 | |
| | | pJHE077 | |
| | | 225 | |
| | | pJHE0109 | |
| | | 225 | |
| | | pJHE0110 | |
| | | 225 | |
| Ernie | pJHE0228, pJHE065, | pJHE0165, pJHE0166 | PEI |
| Transfection | pJHE067 | 225, 225 | |
| | 100, 225, 225 | pJHE0160, pJHE0172 | |
| | | 225, 225 | |
| | | pJHE0186, pJHE0165 | |
| | | 225, 225 | |
| | | pJHE0150, pJHE0172 | |
| | | 225, 225 | |
| | | pJHE0165, pJHE0112 | |
| | | 225, 225 | |
| | | pJHE0160, pJHE0165 | |
| | | 225, 225 | |
| | | pJHE0160, pJHE0195 | |
| | | 225, 225 | |
| | | pJHE0412, pJHE0165 | |
| | | 225, 225 | |
| | | pJHE069, pJHE0150 | |
| | | 225, 225 | |
| | | pJHE0195, pJHE0172 | |
| | | 225, 225 | |
| | | pJHE0168, pJHE0172 | |
| | | 225, 225 | |
| | | pJHE0168, pJHE0195 | |
| | | 225, 225 | |
| | | pJHE0164, pJHE0195 | |
| | | 225, 225 | |
| | | pJHE0164, pJHE0165 | |
| | | 225, 225 | |
| | | pJHE069, pJHE0160 | |
| | | 225, 225 | |
| | | pJHE0160, pJHE0166 | |
| | | 225, 225 | |
| | | pJHE0150, pJHE0112 | |
| | | 225, 225 | |
| | | pJHE0164, pJHE0166 | |
| | | 225, 225 | |
| | | pJHE0412, pJHE0112 | |
| | | 225, 225 | |
| | pJHE0228 | | |
| | 100 | | |
| Big Bird | pJHE0228, jBgB0110 | jBgB047 | jetPRIME |
| Transfection | 50, 225 | 225 | |
| | | jBgB0117 | |
| | | 225 | |
| | | jBgB0153 | |
| | | 225 | |
| | | jBgB0149 | |
| | | 225 | |
| | | jBgB046 | |
| | | 225 | |
| | | jBgB0147 | |
| | | 225 | |
| | | jBgB0156 | |
| | | 225 | |
| | | jBgB0151 | |
| | | 225 | |
| | | jBgB039 | |
| | | 225 | |
| | | jBgB0159 | |
| | | 225 | |
| | | jBgB0152 | |
| | | 225 | |
| | | jBgB0158 | |
| | | 225 | |
| | | jBgB0155 | |
| | | 225 | |
| | | jBgB0150 | |
| | | 225 | |
| | | jBgB036 | |
| | | 225 | |
| | | jBgB0148 | |
| | | 225 | |
| | | jBgB0160 | |
| | | 225 | |
| | | jBgB0157 | |
| | | 225 | |
| | | jBgB0154 | |
| | | 225 | |
| | pJHE0228 | | |
| | 50 | | |
| CONDITION | pJHE0200 | | jetPRIME |
| | 50 | | |
| | pJHE067, pJHE065 | pJHE0200 | |
| | 112.5, 112.5 | 50 | |
| | pJHE067, pJHE065, | pJHE0200 | |
| | pJHE0165, pJHE0166 | 50 | |
| | 112.5, 112.5, | pJHE0460 | |
| | 112.5, 112.5 | 50 | |
| | | pJHE0461 | |
| | | 50 | |
| | | pJHE0462 | |
| | | 50 | |
| | | pJHE0463 | |
| | | 50 | |
| | | pJHE0464 | |
| | | 50 | |
| CONDITION | pJHE0460 | jBgB0108, jBgB047 | PEI |
| | 100 | 450, 450 | |
| | | jBgB0108, jBgB0195 | |
| | | 450, 450 | |
| | | jBgB0110, jBgB047 | |
| | | 450, 450 | |
| | | jBgB0110, jBgB0195 | |
| | | 450, 450 | |
| | | jBgB0108, pJHE0165, | |
| | | pJHE0166 | |
| | | 450, 225, 225 | |
| | | jBgB0110, pJHE0165, | |
| | | pJHE0166 | |
| | | 450, 225, 225 | |
| | | pJHE065, pJHE067, | |
| | | jBgB047 | |
| | | 225, 225, 450 | |
| | | pJHE065, pJHE067, | |
| | | jBgB0195 | |
| | | 225, 225, 450 | |
| | | pJHE065, pJHE067, | |
| | | pJHE0165, pJHE0167 | |
| | | 225, 225, 225, 225 | |

```
SUPPLEMENTARY TABLE 3
Experimental and Transfection Condition by Figure
Details
Cell type: HEK293T
Transfection reagent: PEI
Experimental template: fluorescence plate reader assay
  Transfect at t = 0
  Media exchange at t = 6 h
  At t = 48 h, collect secretion supernatant + lysis samples
Plasmids used:
  Blank (for background subtraction): 1000 ng pcDNA
  Secretion circuits:
    100, 1000 ng secretory reporter
    QS to 1000 ng with pcDNA
Number of replicates per sample: n = 3
Cell type: HEK293T
Transfection reagent: PEI
Experimental template: fluorescence plate reader assay
  Transfect at t = 0
  Media exchange at t = 6 h
  At t = 12 h/24 h/36 h/48 h, collect secretion supernatant + lysis samples. Also
  collect cell counts at each time point
Plasmids used:
  Blank (for background subtraction and as control): 500 ng pcDNA
  Secretion circuit: 100 ng sec-mCh2 + 400 ng pcDNA
Number of replicates per sample: n = 3
Cell type: HEK293T
Transfection reagent: PEI
Experimental template: fluorescence plate reader assay
  Transfect at t = 0
  Media exchange at t = 6 h
  At t = 48 h, collect secretion supernatant
Plasmids used:
    Blank (for background subtraction): 1000 ng pcDNA
    Secretion circuits:
    100 ng sec-mCh2
    FIGS. 2.5A/B:
      450 ng SF 1 (Ernie)
      450 ng SF 2 (Ernie)
    QS to 1000 ng with pcDNA
Number of replicates per sample: n = 3
  I generated the heat maps by pooling the datasets of multiple experiments. To
  accomplish this, I normalized the secretory outputs of each experimental dataset
  to the reporter-only control for that specific experiment. For SF combinations
  repeated across multiple experiments (including reporter-only), I averaged their
  normalized secretory yields across all experiments.
  Due to the large experimental footprints involved (often 16 + 24-well plates), I
  typically conducted 2 + replicate transfections of the reporter-only control to spot
  potential drift in transfection efficiency across the plates (n >= 6). In no cases did
  I notice substantial drift, demonstrating the robustness of these methods.
Synergy calculations with interaction coefficient were conducted using the methods
described by Klumpe et al. (see Chapter 2 for details)
Cell type: HEK293T
Transfection reagent: PEI
Experimental template: fluorescence plate reader assay
  Transfect at t = 0
  Media exchange at t = 6 h
  At t = 48 h, collect secretion supernatant (and lysis samples for Part C)
Plasmids used:
  Blank (for background subtraction): 1000 ng pcDNA
  Secretion circuits:
    100 ng sec-mCh2
    FIG. 2.5C
      900 ng SF (Ernie)
    FIG. 2.5D
      450 ng Big Bird [STXBP1, SF 2]
      450 ng SF 3 (Ernie)
    QS to 1000 ng with pcDNA
Number of replicates per sample: n = 3
  I generated the heat maps by pooling the datasets of multiple experiments. To
  accomplish this, I normalized the secretory outputs of each experimental dataset
  to the reporter-only control for that specific experiment. For SF combinations
  repeated across multiple experiments (including reporter-only), I averaged their
  normalized secretory yields across all experiments.
Cell type: HEK293T
Transfection reagent: PEI
Experimental template: fluorescence plate reader assay
  Transfect at t = 0
  Media exchange at t = 6 h
  At t = 48 h, collect secretion supernatant
Plasmids used:
  Blank (for background subtraction): 1000 ng pcDNA
  Secretion circuits:
100 ng secretory reporter (SP-mCh2)
900 ng pcDNA, or 450 ng STXBP1 + 450 ng SEC24A (Ernies)
Number of replicates per sample: n = 3
Cell type: HEK293T
Transfection reagent: PEI
Experimental template: fluorescence plate reader assay
  Transfect at t = 0
  Media exchange at t = 6 h
  At t = 48 h, collect secretion supernatant (+lysis samples as needed)
Plasmids used:
  Blank (for background subtraction): 1000 ng pcDNA
  Secretion circuits:
    100 ng sec-mCh2 or SP_IL6-mCh2
    Stoichiometrically fixed quantity of Big Bird plasmid (1 to 3 SFs)
      116 fmol of the largest Big Bird [STXBP1, SEC24A, SNAP23] is
      900 ng
    QS to 1000 ng with pcDNA
Number of replicates per sample: n = 3
In Part A, sec-mCh2 and SP_IL6-mCh2 are normalized to the same reference point.
Cell type: HEK293T
Transfection reagent: jetPRIME
Experimental template: fluorescence plate reader assay
  Transfect at t = 0
  Media exchange at t = 6 h
  At t = 48 h, collect secretion supernatant + lysis samples
Plasmids used:
  Blank (for background subtraction): 500 ng pcDNA
  Secretion circuits:
    50 ng sec-mCh2
    Stoichiometrically fixed quantity of Big Bird plasmid (1 to 3 SFs)
      58 fmol of the largest Big Bird [STXBP1, SEC24A, SNAP23] is
      450 ng
    QS to 500 ng with pcDNA
Number of replicates per sample: n = 3
Note that Parts B and C are normalized only to their own internal controls.
Details on Big Bird molar mass and loadings:
```

| Big Bird | Size | ng per | ng for 50 | ng for 58 | ng for 100 | ng for 116 |
|---|---|---|---|---|---|---|
| [. . .] | (kb) | fmol | fmol | fmol | fmol | fmol |
| SEC24A | 7.75 | 4.77 | 239 | 276 | 477 | 553 |
| STXBP1 | 6.25 | 3.85 | 192 | 223 | 385 | 446 |
| SNAP23 | 5.10 | 3.14 | 157 | 182 | 314 | 364 |
| STXBP1 + | 10.76 | 6.62 | 331 | 384 | 662 | 767 |
| SEC24A | | | | | | |
| STXBP1 + | 8.11 | 5.00 | 250 | 289 | 500 | 579 |
| SNAP23 | | | | | | |
| SEC24A + | 9.61 | 5.92 | 296 | 343 | 592 | 685 |
| SNAP23 | | | | | | |
| STXBP1 + | 12.62 | 7.77 | 389 | 450 | 777 | 900 |
| SEC24A + | | | | | | |
| SNAP23 | | | | | | |

Additional mass calculations specific to FIG. 2.9;

| | Size | ng per | target | ng for target |
|---|---|---|---|---|
| Ernie | (kb) | fmol | fmol | fmol |
| miRFP670- | 4.939 | 3.04 | 15 | 46 |
| tagPM | | | | |
| pJH_E0228 | 4.177 | 2.57 | 20 | 51 |
| (sec- | | | | |
| mCherry2) | | | | |

```
Details
Cell type: HEK293T
Transfection reagent: jetPRIME
Experimental template: super-resolution fluorescence microscopy
  Transfect at t = 0
  Media exchange at t = 6 h
  At t = 24 h, replate cells into Ibidi chamber slides
  Overnight incubation with CellLight Golgi-GFP prior to imaging (t = ~32 h, or 16 h
  prior to imaging per manufacturer instructions)
    Follow manufacturer dosing protocol (~10 μL for 50k cells)
  Incubate cells with ER-Tracker Blue-White DPX ~1 h prior to imaging
    Follow manufacturer dosing protocol (100 nM working concentration)
  At t = 48 h, image live cells using microscope
Plasmids used:
  20 fmol of sec-mCh2 (51 ng) secretory reporter
  15 fmol of miRFP670-tag_{PM} (46 ng) plasma membrane tag
  50 fmol of SF Big Bird (up to 389 ng)
  QS to 500 ng with pcDNA
Number of replicates per sample: N/A
Cell type: HEK293T (for LV producer and as LV target)
Reagents:
  jetPRIME transfection of HEK293T to produce lentivirus
  LV-laden supernatant media for transduction of other HEK293T cells
Experimental template: flow cytometry
  At t = 0, transfect HEK293T cells with lentivirus assembly plasmids (and our SF
  program)
  At t = 4 h, media exchange
  At t = 24 h, harvest secretion LV supernatant and refrigerate
  At t = 48 h, repeat secretion LV supernatant harvesting
  Pool the Day 1 and Day 2 supernatant samples and conduct viral filtration
  Transduce plated HEK293T cells using lentivirus (50% dilution into culture media,
  with polybrene added for a working concentration of 8 ug/mL). 48h later, run cells
  on flow cytometer
Transfection notes:
  System is 2^{nd}-generation lentivirus
  Cells were transfected in a 24-well format to produce viral vector
Plasmids used for transfection (per well):
  43.75 ng eGFP LV expression payload (pOCF222)
  93.75 ng packaging plasmid (psPAX2)
  62.5 ng envelope plasmid (pMD2R)
  300 ng of 3-SF Big Bird [STXBP1, SEC24A, SNAP23] or pcDNA (control)
Number of replicates per sample: varies at each stage
  Transfection: n = 3
    Each replicate is split across 4 wells in a 24-well format
  Transduction: n = 3 × 3 = 9
    Each of the 3 transfection replicates is transduced into 3 replicate wells
Cell type: HEK293T
Transfection reagent: PEI
Experimental template: fluorescence plate reader assay
  Transfect at t = 0
  Media exchange at t = 6 h
  At t = 48 h, collect secretion supernatant
Plasmids used:
  Blank (for background subtraction): 1000 ng pcDNA
  Secretion circuits:
    100 ng secretory reporter
    900 ng of the 2-SF Big Bird [STXBP1, SEC24A] or 900 ng pcDNA
Number of replicates per sample: n = 3
Cell type: HEK293T
Transfection reagent: PEI
Experimental template: absorbance plate reader assay (trastuzumab antibody)
  Transfect at t = 0
  Media exchange at t = 6 h
  At t = 48 h, collect secretion supernatant
    Use the Easy-Titer human IgG (H + L) assay kit (Thermo Fisher)
  Prepare a standard curve (using pure trastuzumab) per manufacturer instructions
  Dilute and run secretion samples; fit data points to standard curve for mAb yield
  estimate
Plasmids used:
  100 ng of the 2-gene Big Bird [LC, HC] encoding trastuzumab
  900 ng of the 3-SF Big Bird [STXBP1, SEC24A, SNAP23] or 900 ng pcDNA
Number of replicates per sample: n = 3
Cell type: hUC-MSCs
Transfection reagent: Lonza 4D Nucleofector (P1 Primary Cell Kit)
Experimental template: ELISA plate reader assay
  Frozen cells were thawed and washed to remove DMSO
  Cells were resuspended in P1 primary cell buffer mixed with plasmids
  At t = 0, cells were electroporated using the Nucleofector
  Cells were split into equal volumes and incubated until supernatant harvest and
  cell count at t = 48 h. Cells were also evaluated for transfection efficiency using
  flow cytometry
  Secreted cytokine IL-10 concentration was determined using ELISA
Plasmids used (per million cells):
  500 ng BFP
  0, 500 ng human IL-10 (CMV promoter)
  0, 250, 500, 1000 ng of our 3-SF Big Bird [STXBP1, SEC24A, SNAP23]
  QS to 2000 ng using pcDNA
Number of replicates per sample: n = 3 (single transfection, split to 3 flasks)
Cell type: ARPE-19
Transfection reagent: Lipofectamine 3000
Experimental template: fluorescence plate reader assay
  Transfect at t = 0
  Media exchange at t = 6 h
  At t = 48 h, collect secretion supernatant + lysis samples
Plasmids used:
  Blank (for background subtraction): 500 ng pcDNA
  Secretion circuits:
    50 ng of the secretory reporter SP_IL6-mCh2
    450 ng of the 3-SF Big Bird [STXBP1, SEC24A, SNAP23] or 900 ng
    pcDNA filler
Number of replicates per sample: n = 3
```

TABLE 3
Chapter 2 experimental details. Where indicated, “pcDNA” refers to blank plasmid
based on the pcDNA vector used for mass balance purposes during specific transfections.
Details

```
Cell type: HEK293T
Transfection reagent: PEI
Experimental template: live-cell confocal microscopy
Plasmids used:
  100 ng of fluorescent protein (eGFP, CHGA_{Mus}-eGFP/sfGFP/mCh2)
  0, 100 ng of each SF Ernie used
  QS to 500 ng with pcDNA
Number of replicates per sample: N/A
Cell type: HEK293T
Transfection reagent: PEI
Experimental template: fluorescence plate reader assay
  Transfect at t = 0
  Media exchange at t = 6 h
  At t = 48 h, media exchange (+/−ionophore)
  3 h later, collect secretion supernatant
Plasmids used:
  Blank (for background subtraction): 1000 ng pcDNA
  Secretory circuits:
    100 ng of CHGA-mCh2
    225 ng of each SF Ernie screened (up to 4)
    QS to 1000 ng with pcDNA
Number of replicates per sample: n = 3
  I generated the heat maps by pooling the datasets of multiple experiments. To
  accomplish this, I normalized the secretory outputs of each experimental dataset
  to the reporter-only control for that specific experiment. For SF combinations
  repeated across multiple experiments (including reporter-only), I averaged their
  normalized secretory yields across all experiments.
Cell type: HEK293T
Transfection reagent: PEI
Experimental template: fluorescence plate reader assay
  Transfect at t = 0
  Media exchange at t = 6 h
  At t = 48 h, media exchange (+/−ionophore)
  3 h later, collect secretion supernatant
Plasmids used:
  Blank (for background subtraction): 1000 ng pcDNA
  Secretory circuits:
    100 ng of CHGA-mCh2
    225 ng of each SF Ernie screened (up to 4)
    QS to 1000 ng with pcDNA
Number of replicates per sample: n = 3
  The data set shown here was compiled from the same heatmap experiments used
  to construct FIGS. 3.3 and 3.4. As a result, some conditions were repeated
  multiple times throughout these experiments: normalized secretory values across
  all runs analyzed were factored into the reported average values
Cell type: HEK293T
Transfection reagent: jetPRIME
Experimental template: fluorescence plate reader assay
  Transfect at t = 0
  Media exchange at t = 6 h
  At t = 48 h, media exchange (+/−ionophore)
  3 h later, collect secretion supernatant
Plasmids used:
  Blank (for background subtraction): 500 ng pcDNA
  Secretory circuits:
    50 ng of CHGA-mCh2
    225 ng of each 2-SF Big Bird screened (up to 2)
    QS to 500 ng with pcDNA
Number of replicates per sample: n = 3
  Data generated from a single experimental run
Cell type: HEK293T
Transfection reagent: jetPRIME
Experimental template: fluorescence plate reader assay
  Transfect at t = 0
  Media exchange at t = 6 h
  At t = 48 h, media exchange (+/−ionophore)
  3 h later, collect secretion supernatant
Plasmids used:
  Blank (for background subtraction): 500 ng pcDNA
  Secretory circuits:
    50 ng of CHGA-mCh2 or CHGA-IDR-mCh2 (IDR1-5)
    112.5 ng of each SF Ernie screened (up to 4)
    QS to 500 ng with pcDNA
Number of replicates per sample: n = 3
Cell type: HEK293T
Transfection reagent: jetPRIME
Experimental template: fluorescence plate reader assay
  Transfect at t = 0
  Media exchange at t = 6 h
  At t = 48 h:
    Regulated secretory induction:
      Media exchange (+/−ionophore)
      3 h later, collect secretion supernatant
    Also, collect constitutive secretion supernatant + lysis samples
  This must be done with identical transfection replicates
    Total of 9 replicate wells per condition
    6 wells for 3 h inductions, 3 wells for constitutive samples
Plasmids used:
  Blank (for background subtraction): 500 ng pcDNA
  Secretory circuits:
    50 ng of CHGA-mCh2 or CHGA-IDR-mCh2 (IDR1)
    112.5 ng of each SF Ernie screened (up to 4)
    QS to 500 ng with pcDNA
Number of replicates per sample: n = 3
Cell type: HEK293T
Transfection reagent: jetPRIME
Experimental template: super-resolution fluorescence microscopy
  Transfect at t = 0
  Media exchange at t = 6 h
  At t = 24 h, replate cells into Ibidi chamber slides
  At t = 48 h, image live cells using microscope
    Initial well volume is 100 μL
    To add ionophore at 1x working concentration without disturbing the cell
    bed, add 10 μL of 11x working concentration of FluoroBrite DMEM
    DMEM added must be serum-free to avoid cell response to fresh FBS
Plasmids used:
  50 ng of CHGA-mCh2 or CHGA-IDR-mCh2 (IDR1)
  112.5 ng of each SF Ernie screened (up to 4)
  QS to 500 ng with pcDNA
Number of replicates per sample: N/A
Cell type: HEK293T
Transfection reagent: PEI
Experimental template: fluorescence plate reader assay
  Transfect at t = 0
  Media exchange at t = 6 h
  At t = 48 h, media exchange (+/−ionophore)
  3 h later, collect secretion supernatant
Plasmids used:
  Blank (for background subtraction): 1000 ng pcDNA
  Secretory circuits (up to 4 SFs):
    100 ng of CHGA-mCh2
    225 ng of each SF Ernie screened
    450 ng of each 2-SF Big Bird screened
    QS to 1000 ng with pcDNA
Number of replicates per sample: n = 2
Cell type: HEK293T
Transfection reagent: PEI
Experimental template: fluorescence plate reader assay
  Transfect at t = 0
  Media exchange at t = 6 h
  At t = 48 h, media exchange (+/−ionophore)
  3 h later, collect secretion supernatant
Plasmids used:
  Blank (for background subtraction): 1000 ng pcDNA
  Secretory circuits:
    100 ng of CHGA-mCh2
    Titrated 4-SF secretory programs:
      SYT7, STX4 as Ernies (175/200/225 ng each)
      2-SF Big Bird [RAB27B, RAB26] (350/400/450 ng)
    Varied pcDNA loading to simulate addition circuit elements
Number of replicates per sample: n = 2
```

TABLE 4
Chapter 3 experimental details. Where indicated, “pcDNA” refers to blank plasmid
based on the pcDNA vector used for mass balance purposes during specific transfections.
Details

```
Cell type: HEK293T
Transfection reagent: PEI
Experimental template: flow cytometry
  Transfect at t = 0
  Media exchange at t = 6 h
  Add ionophore at t = 24 h
  Live cell flow cytometry at t = 48 h
Plasmids used:
  BFP-only control: 200 ng BFP + 800 ng pcDNA
  Transcriptional reporter variants:
    Transfection efficiency reporter: 200 ng BFP
    Ca²⁺ reporter: 400 ng pNFAT-sfGFP
    Exogenous NFAT: 0, 10, 50 ng NFAT4
    pcDNA filler: QS to 1000 ng
Number of replicates per sample: n = 1
Cell type: HEK293T
Transfection reagent: PEI
Experimental template: flow cytometry
  Transfect at t = 0
  Media exchange at t = 6 h
  Add ligand (rapalog, ionophore) at t = 24 h
  Live cell flow cytometry at t = 48 h
Plasmids used:
  BFP-only control: 200 ng BFP + 800 ng pcDNA
  Circuit variants:
    Transcriptional reporter system:
      Transfection efficiency reporter: 200 ng BFP
      Ca²⁺ reporter: 300 ng of 9x(pIL8)-sfGFP
      Exogenous NFAT: 50 ng NFAT4
    SOAR-Orai circuit:
      0, 250 ng Myc-SOAR-FRB
      0, 250 ng HA-Orai1-FKBP
    pcDNA filler: QS to 1050 ng
Number of replicates per sample: n = 1
Cell type: HEK293T
Transfection reagent: PEI
Experimental template: fluorescence plate reader assay
  Transfect at t = 0
  Media exchange at t = 6 h
  At t = 48 h, media exchange (+/−ionophore or rapalog)
    Deviation from SOP: used 4x greater rapalog than standard
  3 h later, collect secretion supernatant
Plasmids used:
  Blank (for background subtraction): 1300 ng pcDNA
  Sense-and-secrete circuit variants:
    Regulated secretion module:
      100 ng CHGA-IDR-mCh2
      200 ng SYT7
      200 ng STX4
      400 ng Big Bird [RAB27B, RAB26]
    SOAR-Orai circuit:
      0, 200 ng Myc-SOAR-FRB
      0, 200 ng HA-Orai1-FKBP
    pcDNA filler: QS to 1300 ng
Number of replicates per sample: n = 2
Cell type: HEK293T
Transfection reagent: PEI
Experimental template: flow cytometry
  Transfect at t = 0
  Media exchange at t = 6 h
  Add ligand (rapalog, ionophore) at t = 24 h
  Live cell flow cytometry at t = 48 h
Plasmids used:
  BFP-only control: 200 ng BFP + 1000 ng pcDNA
  Circuit variants:
    Transcriptional reporter system:
      Transfection efficiency reporter: 200 ng BFP
      Ca²⁺ reporter: 300 ng of 9x(pIL8)-sfGFP
      Exogenous NFAT: 50 ng NFAT4
    SOAR-Orai circuit:
      0, 100 ng Myc-SOAR-FKBP
      0, 100 ng Myc-SOAR-FRB
      0, 25, 50, 100 ng HA-Orai1
    pcDNA filler: QS to 1200 ng
Number of replicates per sample: n = 1
Cell type: HEK293T
Transfection reagent: PEI
Experimental template: fluorescence plate reader assay
  Transfect at t = 0
  Media exchange at t = 6 h
  At t = 48 h, media exchange (+/−ionophore or rapalog)
  3 h later, collect secretion supernatant
Plasmids used:
  Blank (for background subtraction): 1200 ng pcDNA
  Sense-and-secrete circuit variants:
    Regulated secretion module:
      100 ng CHGA-IDR-mCh2
      200 ng SYT7
      200 ng STX4
      400 ng Big Bird [RAB27B, RAB26]
    SOAR-Orai circuit:
      0, 100 ng Myc-SOAR-FKBP
      0, 100 ng Myc-SOAR-FRB
      0, 25, 50, 100 ng HA-Orai1
    pcDNA filler: QS to 1200 ng
Number of replicates per sample: n = 2
Cell type: HEK293T
Transfection reagent: PEI
Experimental template: flow cytometry
  Transfect at t = 0
  Media exchange at t = 6 h
  Add ligand (EPO, ionophore) at t = 24 h
  Live cell flow cytometry at t = 48 h
Plasmids used:
  BFP-only control: 200 ng BFP + 800 ng pcDNA
  Circuit variants:
    Transcriptional reporter system:
      Transfection efficiency reporter: 200 ng BFP
      Ca²⁺ reporter: 300 ng of 9x(pIL8)-sfGFP
      Exogenous NFAT: 50 ng NFAT4
    SOAR-Orai circuit:
      0, 250 ng HA-EpoR-CD3Z
      0, 50 ng JAK2
      0, 300 ng Myc-SOAR-SH2
      0, 20, 50, 100 ng Orai1
      0, 50, 100 ng PTPN1-Zip
    pcDNA filler: N/A (except for reporter-only + ionophore-QS to 1050 ng)
Number of replicates per sample: n = 1
Cell type: HEK293T
Transfection reagent: PEI
Experimental template: fluorescence plate reader assay
  Transfect at t = 0
  Media exchange at t = 6 h
  At t = 48 h, media exchange (+/−ionophore or EPO)
  3 h later, collect secretion supernatant
Plasmids used:
  Blank (for background subtraction): 1000 ng pcDNA
  Sense-and-secrete circuit variants:
    Regulated secretion module:
      100 ng CHGA-IDR-mCh2
      175 ng SYT7
      175 ng STX4
      350 ng Big Bird [RAB27B, RAB26]
    SOAR-Orai circuit:
      0, 250 ng HA-EpoR-CD3Z
      0, 50 ng JAK2
      0, 300 ng Myc-SOAR-SH2
      0, 20, 100 ng Orai1
      0, 100 ng PTPN1-Zip
    pcDNA filler: N/A (except for ionophore positive control-QS to 1400 ng)
Number of replicates per sample: n = 2
```

Supplementary Table 4.
Amino Acid Sequences of key effector proteins used in this study

| Construct/ | |
|---|---|
| domain ID | Amino acid coding sequence |
| mCh2 | MVSKGEENNLAIIKEFMRFKVHMEGSVNGHEFEIEGEGEGRPYEGTQTAKLKVT |
| (D1-CMV-_{K}) | KGGPLPFAWDILSPQFMYGSKAYVKHPADIPDYLKLSFPEGFNWERVMNFEDG |
| (BGH225) | GVVTVTQDSSLQDGEFIYKVKLRGTNFPSDGPVMQCRTMGWEASTERMYPED |
| | GALKGEIKQRLKLKDGGHYDAEVKTTYKAKKPVQLPGAYNVDIKLDILSHNEDYTI |
| | VEQYERAEGRHSTGGMDELYK SEQ ID NO: 53 |
| Signal | “Signal peptide” (uses subsequent sequence “GS” to host Type IIS |
| peptide (SP) | cleavage site) |
| (Individual | Signal sequence variants: |
| domain-not | Secrecon (sec): MWWRLWWLLLLLLLLWPMVWA SEQ ID NO: 13 |
| a complete | Interleukin-6 (IL6): MNSFSTSAFGPVAFSLGLLLVLPAAFPAP SEQ ID NO: 1 |
| construct) | Interleukin-2 (IL2): MQLLSCIALILALV SEQ ID NO: 2 |
| (Modular N- | Trypsin (TRY1) + propeptide: MNPLLILTFVAAALAAPFDDDDK SEQ ID NO: 21 |
| terminal tag | Gaussia luciferase: MGVKVLFALICIAVAEA SEQ ID NO: 3 |
| for secreted | Chromogranin A (CHGA): MRSAAVLALLLCAGQVTA SEQ ID NO: 4 |
| proteins) | Factor BIII (F8): MQIELSTCFFLCLLRFCFS SEQ ID NO: 10 |
| | Albumin (ALB): MKWVTFISLLFLFSSAYS SEQ ID NO: 6 |
| | Anti-HER2 heavy chain: MGWSLILLFLVAVATRVHS SEQ ID NO: 7 |
| | Anti-HER2 light chain: MRVPAQLLGLLLLWLPGARC SEQ ID NO: 8 |
| | Erythropoietin (EPO): MGVHECPAWLWLLLSLLSLPLGLPVLG SEQ ID NO: 9 |
| | Secretogranin III (SCG3): MGFLGTGTWILVLVLPIQA SEQ ID NO: 19 |
| | Factor IX (F9): MQRVNMIMAESPGLITICLLGYLLSAEC SEQ ID NO: 11 |
| | VGF nerve growth factor inducible: MKALRLSASALFCLLLINGLGA SEQ ID NO: 20 |
| | Secretogranin II (SCG2): MAEKAKTHWLGAALSLIPLIFLISGAEA SEQ DI NO: 18 |
| | Neuroendocrine protein 7B2 (SCG5): MVSRMVSTMLSGLLFWLASGWTPAFA SEQ ID NO: 17 |
| | Chromogranin B (CHGB): MQPTLLLSLLGAVGLAAVNS SEQ ID NO: 5 |
| | Hidden Markov Model (HMM), Score of 34: MRPTWAWWLFLVLLLALWAPARG SEQ ID NO: 16 |
| | IgG heavy chain (GenBank: AAA52897): MDWTWRVFCLLAVTPGAH SEQ ID NO: 15 |
| | Interferon alpha-2 (IFNA2): MALTFALLVALLVLSCKSSCSVG SEQ ID NO: 12 |
| | Vasopressin (SP_AVP): MPDTMLPACFLGLLAFSSA SEQ ID NO: 14 |
| SP-mCh2 | “Signal peptide”-GS |
| (D1-CMV-_{K}) | MVSKGEENNL . . . STGGMDELYK SEQ ID NOS 54 and 55 |
| (BGH225) | |
| SP-EPO- | “Signal peptide”-GS |
| mCh2 | APPRLICDSRVLERYLLEAKEAENITTGCAEHCSLNENITVPDTKVNFYAWKRMEVGQQAVEVWQGLALLSEAVLRG |
| (D1-CMV-_{K}) | QALLVNSSQPWEPLQLHVDKAVSGLRSLTTLLRALGAQKEAISPPDAASAAPLRTITADTFRKLFRVYSNFLRGKLK |
| (BGH225) | LYTGEACRTGDR SEQ ID NO: 56 |
| | GSGSGSGS SEQ ID NO: 22 |
| | MVSKGEENNL . . . STGGMDELYK SEQ ID NOS 54 and 56 |
| SP-FA9- | “Signal peptide”-GS |
| mCh2 | YNSGKLEEFVQGNLERECMEEKCSFEEAREVFENTERTTEFWKQYVDGDQCESNPCLNGGSCKDDINSYECWCPFGF |
| (D1-CMV-_{K}) | EGKNCELDVTCNIKNGRCEQFCKNSADNKVVCSCTEGYRLAENQKSCEPAVPFPCGRVSVSQTSKLTRAETVFPDVD |
| (BGH225) | YVNSTEAETILDNITQSTQSFNDFTRVVGGEDAKPGQFPWQVVLNGKVDAFCGGSIVNEKWIVTAAHCVETGVKITV |
| | VAGEHNIEETEHTEQKRNVIRIIPHHNYNAAINKYNHDIALLELDEPLVLNSYVTPICIADKEYTNIFLKFGSGYVS |
| | GWGRVFHKGRSALVLQYLRVPLVDRATCLRSTKFTIYNNMFCAGFHEGGRDSCQGDSGGPHVTEVEGTSFLTGIISW |
| | GEECAMKGKYGIYTKVSRYVNWIKIKTKLT SEQ ID NO: 57 |
| | GSGSGSGS SEQ ID NO: 22 |
| | MVSKGEENNL . . . STGGMDELYK SEQ ID NOS 54 and 55 |
| CHGA-mCh2 | MRSAAVLALLLCAGQVTALPVNSPMNKGDTEVMKCIVEVISDTLSKPSPMPVSQECFETLRGDERILSILRHQNLLK |
| (D1-CMV-_{K}) | ELQDLALGQAKERAHQQKKHSGFEDELSEVLENQSSQAELKEAVEEPSSKDVMEKREDSKEAEKSGEATDGARPQAL |
| (BGH225) | PEPMQESKAENGNNQAPGEEEEEEEEATNTHPPASLPSQKYPGPQAEGDSEGLSQGLVDREKGLSAEPGWQAKREEE |
| | EEEEEEAEAGEEAVPEEEGPTVVLNPHPSLGYKEIRKGESRSEALAVDGAGKPGAEEAQDPEGKGEQEHSQQKEEEE |
| | EMAVVPQGLFRGGKSGELEQEEERLSKEWEDSKRWSKMDQLAKELTAEKRLEGQEEEEDNRDSSMKLSFRARAYGFR |
| | GPGPQLRRGWRPSSREDSLEAGLPLQVRGYPEEKKEEEGSANRRPEDQELESLAIEAELEKVAHQLQALRRG |
| | SEQ ID NO: 58 |
| | GSGSGSGS SEQ ID NO: 22 |
| | MVSKGEENNL . . . STGGMDELYK SEQ ID NOS 54 and 55 |
| CHGA-IDR- | MRSAAVLALL . . . AHQLQALRRG SEQ ID NOS 59 and 60 |
| mCh2 | GSG-“IDR domain”-GS |
| (D1-CMV-_{K}) | MVSKGEENNL . . . STGGMDELYK SEQ ID NOS 54 and 55 |
| (BGH225) | IDR sequence variants: |
| | IDR1 (default): ESNQSNNGGSGNAALNRGGRYVPPHLRGGS SEQ ID NO: 61 |
| | IDR2: SAGGDDRRGGAGGGGYRRGGGNS SEQ ID NO: 62 |
| | IDR3: YNGGGGGGGNRGYNNNRGGGGGGYN SEQ ID NO: 63 |
| | IDR4: ESNQSNNGGSGNAALNRGGRYVPPHLRGGSSAGGDDRRGGAGGGGYRRGGGNS SEQ ID NO: 64 |
| | IDR5: ESNQSNNGGSGNAALNRGGRYVPPHLRGGSSAGGDDRRGGAGGGGYRRGGGNSYNGGGGGGGNRGYNNNRG |
| | GGGGGYN SEQ ID NO: 65 |
| SOAR | SSWYAPEALQKWLQLTHEVEVQYYNIKKQNAEKQLLVAKEGAEKIKKKRNTLFGTFHVAHSSSLDDVDHKILTAKQA |
| (Individual | LSEVTAALRERLHRWQQIEILCGFQIVNNPGIH SEQ ID NO: 66 |
| domain-not | IDR sequence variants: |
| a complete | A369 → V/D/K/S/G |
| construct) | L373 → A/V/S |
| | A376 → D/K/S/G |
| | A380 → D/K/S/G |
| Myc-SOAR- | MVELKRSEEQKLISEEDLLRSEEQKLISEEDLLRSEEQKLISEEDLLGNS SEQ ID NO: 67 |
| FRB | SSWYAPEALQ . . . FQIVNNPGIH SEQ ID NOS 68 and 69 |
| (D1-CMV) | GTGSGSGSGS SEQ ID NO: 70 |
| (BGH225) | ILWHEMWHEGLEEASRLYFGERNVKGMFEVLEPLHAMMERGPQTLKETSFNQAYGRDLMEAQEWCRKYMKSGNVKDL |
| (Pre-SS) | LQAWDLYYHVFRRISK SEQ ID NO: 71 |
| Myc-SOAR- | MVELKRSEEQKLISEEDLLRSEEQKLISEEDLLRSEEQKLISEEDLLGNS SEQ ID NO: 67 |
| FKBP | SSWYAPEALQ . . . FQIVNNPGIH SEQ ID NOS 68 and 69 |
| (D1-CMV) | GTGSGSGSGS SEQ ID NO: 70 |
| (BGH225) | GVQVETISPGDGRTFPKRGQTCVVHYTGMLEDGKKFDSSRDRNKPFKFMLGKQEVIRGWEEGVAQMSVGQRAKLTIS |
| (Pre-SS) | PDYAYGATGHPGIIPPHATLVFDVELLKLE SEQ ID NO: 93 |
| HA-Orai | MGNCSYPYDVPDYAGSYPYDVPDYAGSYPYDVPDYAGENS SEQ ID NO: 75 |
| (D1-CMV) | MHPEPAPPPSRSSPELPPSGGSTTSGSRRSRRRSGDGEPPGAPPPPPSAVTYPDWIGQSYSEVMSLNEHSMQALSWR |
| (BGH225) | KLYLSRAKLKASSRTSALLSGFAMVAMVEVQLDADHDYPPGLLIAFSACTTVLVAVHLFALMISTCILPNIEAVSNV |
| (Pre-SS) | HNLNSVKESPHERMHRHIELAWAFSTVIGTLLFLAEVVLLCWVKFLPLKKQPGQPRPTSKPPASGAAANVSTSGITP |
| | GQAAAIASTTIMVPFGLIFIVAVHFYRSLVSHKTDRQFQELNELAEFARLQDQLDHRGDHPLTPGSHYA |
| | SEQ ID NO: 72 |
| Orai | MHPEPAPPPS . . . HPLTPGSHYA SEQ ID NOS 73 and 74 |
| (D1-CMV) | |
| (BGH225) | |
| (Pre-SS) | |
| HA-Orai1- | MGNCSYPYDVPDYAGSYPYDVPDYAGSYPYDVPDYAGENS SEQ ID NO: 75 |
| FKBP | MHPEPAPPPS . . . HPLTPGSHYA SEQ ID NO 73 and 74 |
| (D1-CMV) | GTGSGSGSGS SEQ ID NO: 70 |
| (BGH225) | GVQVETISPG . . . VFDVELLKLE SEQ ID NOS 76 and 77 |
| (Pre-SS) | |
| Myc-SOAR- | MAVELKRSEEQKLISEEDLLRSEEQKLISEEDLLRSEEQKLISEEDLLGNS SEQ ID NO: 78 |
| (SH2)₂ | SSWYAPEALQ . . . FQIVNNPGIH SEQ ID NOS 68 and 69 |
| (D1-CMV-β) | GSGSGSGSGSGSSGSGSGGSSGSGGSSSGGSGSGSGSSGGSGGGGSGGSSGSSSGGGGS SEQ ID NO: 79 |
| (BGH225) | PDPAAHLPFFYGSISRAEAEEHLKLAGMADGLFLLFQCLRSLGGYVLSLVHDVRFHHFPIERQLNGTYAIAGGKAHC |
| | GPAELCEFYSRPDPGLPCNLRKPCNRPSGLEPQPGVFDCLRDAMVRDYVRQTWKLEGEALEQAIISQAPQVEKLIAT |
| | TAHERMPWYHSSLTREEAERKLYSGAQTDGKFLLRPRKEQGTYALSLIYGKTVYHYISQDKAGKYCIPEGTKFDTLW |
| | QLVEYLKLKADGLIYCLKEACPNSSA SEQ ID NO: 80 |
| | GSGSGSGSGSGSGSGSGSGS SEQ ID NO: 81 |
| | PDPAA . . . PNSSA SEQ ID NOS 82 and 83 |

### EQUIVALENTS

Those skilled in the art will recognize, or be able to ascertain, using no more than routine experimentation, numerous equivalents to the specific substances and procedures described herein. Such equivalents are considered to be within the scope of this invention and are covered by the following claims.

We claim:

1. A genetically engineered cell comprising an expression vector, wherein the expression vector encodes for two or more recombinant proteins selected from the group consisting of STXBP1, SNAP23, SNAP25, SNAP91, VAMP2, VAMP7, BNIP1, GOSR1, BET1, SEX22B, YKT6, SCFD1, SAR1B, SEC24A, and STXBP1.

2. The genetically engineered cell of claim 1, wherein the two or more recombinant protects comprises STXBP1, SEC24A, and SNAP23.

3. The genetically engineered cell of claim 1, wherein the two or more recombinant proteins are expressed from one or more plasmids or from a multi-gene plasmid.

4. The genetically engineered cell of claim 1, wherein the cell comprises a mammalian cell.

5. The genetically engineered cell of claim 4, wherein the mammalian cell comprises a HEK293T cell, a retinal pigment epithelial cell, or a mesenchymal stem cell.

6. The genetically engineered cell of claim 1, wherein the cell comprises an encapsulated cell.

7. The genetically engineered cell of claim 1, wherein the cell is engineered to express and/or secrete a biomolecule of interest.

8. The genetically engineered cell of claim 7, wherein the biomolecule of interest comprises a recombinant protein or polypeptide or a nucleic acid.

9. The genetically engineered cell of claim 8, wherein the recombinant protein or polypeptide comprises a prohormone, a hormone, a blood clotting protein, an antibody or antigen binding fragment thereof, a protein receptor, a fusion protein, a vaccine, a cytokine, a chemokine, a growth factor, or a blood factor protein.

10. The generically engineered cell of claim 8, wherein the nucleic acid comprises a recombinant virus or viral vector.

11. A vector encoding two or more recombinant proteins selected from the group consisting of STXBP1, SNAP23, SNAP25, SNAP91, VAMP2, VAMP7, BNIP1, GOSR1, BET1, SEX22B, YKT6, SCFD1, SAR1B, SEC24A, and STXBP1.

12. The vector of claim 11, wherein the two or more recombinant protects comprises STXBP1, SEC24A, and SNAP23.

13. The vector of claim 11, wherein the vector comprises one or more additional regulatory elements.

14. A cell comprising the vector of claim 11.

15. The cell of claim 14, wherein the cell comprises a mammalian cell.

16. The cell of claim 15, wherein the mammalian cell comprises a HEK293T cell, a retinal pigment epithelial cell, or a mesenchymal stem cell.

17. The cell of claim 14, wherein the cell comprises an encapsulated cell.

18. A method for producing a biomolecule of interest, said method comprising
culturing a population of mammalian cells, wherein the population of mammalian cells are genetically engineered to express two or more recombinant proteins selected from the group consisting of STXBP1, SNAP23, SNAP25, SNAP91, VAMP2, VAMP7, BNIP1, GOSR1, BET1, SEX22B, YKT6, SCFD1, SAR1B, SEC24A, and STXBP1, wherein the population of mammalian cells are further genetically engineered to express the biomolecule; and
recovering said biomolecule of interest from the medium from said culture.

19. The method of claim 18, wherein the two or more recombinant proteins synergize to increase production and/or secretion of the biomolecule.

20. The method of claim 18, further comprising transducing the population of mammalian cells with one or more vectors expressing the two or more recombinant proteins and/or the biomolecule.

21. A genetically engineered cell comprising a synthetic secretory pathway, wherein the synthetic secretory pathway comprises a recombinant membrane receptor configured to sense a signaling compound, a recombinant calcium ion channel, and a recombinant secretion component, wherein the membrane receptor and the recombinant secretion component are operably linked by the recombinant calcium ion channel.

22. The genetically engineered cell of claim 21, wherein the cell comprises a mammalian cell.

23. The genetically engineered cell of claim 22, wherein the mammalian cell comprises a HEK293T cell, a retinal pigment epithelial cell, or a mesenchymal stem cell.

24. The genetically engineered cell of claim 21, wherein the cell comprises an encapsulated cell.

25. The genetically engineered cell of claim 21, wherein the cell comprises an expression vector encoding for two or more recombinant proteins selected from the group consisting of SYT7, STX4, RAB27A, and RAB27B.


SEQUENCE LISTING (93 sequences)

SEQ ID NO: 1 - protein, 29 aa, synthetic construct
MNSFSTSAFGPVAFSLGLLLVLPAAFPAP

SEQ ID NO: 2 - protein, 14 aa, synthetic construct
MQLLSCIALILALV

SEQ ID NO: 3 - protein, 17 aa, synthetic construct
MGVKVLFALICIAVAEA

SEQ ID NO: 4 - protein, 18 aa, synthetic construct
MRSAAVLALLLCAGQVTA

SEQ ID NO: 5 - protein, 20 aa, synthetic construct
MQPTLLLSLLGAVGLAAVNS

SEQ ID NO: 6 - protein, 18 aa, synthetic construct
MKWVTFISLLFLFSSAYS

SEQ ID NO: 7 - protein, 19 aa, synthetic construct
MGWSLILLFLVAVATRVHS

SEQ ID NO: 8 - protein, 20 aa, synthetic construct
MRVPAQLLGLLLLWLPGARC

SEQ ID NO: 9 - protein, 27 aa, synthetic construct
MGVHECPAWLWLLLSLLSLPLGLPVLG

SEQ ID NO: 10 - protein, 19 aa, synthetic construct
MQIELSTCFFLCLLRFCFS

SEQ ID NO: 11 - protein, 28 aa, synthetic construct
MQRVNMIMAESPGLITICLLGYLLSAEC

SEQ ID NO: 12 - protein, 23 aa, synthetic construct
MALTFALLVALLVLSCKSSCSVG

SEQ ID NO: 13 - protein, 21 aa, synthetic construct
MWWRLWWLLLLLLLLWPMVWA

SEQ ID NO: 14 - protein, 19 aa, synthetic construct
MPDTMLPACFLGLLAFSSA

SEQ ID NO: 15 - protein, 18 aa, synthetic construct
MDWTWRVFCLLAVTPGAH

SEQ ID NO: 16 - protein, 23 aa, synthetic construct
MRPTWAWWLFLVLLLALWAPARG

SEQ ID NO: 17 - protein, 26 aa, synthetic construct
MVSRMVSTMLSGLLFWLASGWTPAFA

SEQ ID NO: 18 - protein, 27 aa, synthetic construct
MAEAKTHWLGAALSLIPLIFLISGAEA

SEQ ID NO: 19 - protein, 19 aa, synthetic construct
MGFLGTGTWILVLVLPIQA

SEQ ID NO: 20 - protein, 22 aa, synthetic construct
MKALRLSASALFCLLLINGLGA

SEQ ID NO: 21 - protein, 23 aa, synthetic construct
MNPLLILTFVAAALAAPFDDDDK

SEQ ID NO: 22 - protein, 8 aa, synthetic construct
GSGSGSGS

SEQ ID NO: 23 - protein, 756 aa, synthetic construct
MAVELKRSEEQKLISEEDLLRSEEQKLISEEDLLRSEEQKLISEEDLLGNSSSWYAPEALQKWLQLTHEVEVQYYNIKKQNAEKQLLVAKEGAEKIKKKRNTLFGTFHVAHSSSLDDVDHKILTAKQALSEVTAALRERLHRWQQIEILCGFQIVNNPGIHGSGSGSGSGSGSSGSGSGGSSGSGGSSSGGSGSGSGSSGGSGGGGSGGSSGSSSGGGGSPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSAGSGSGSGSGSGSGSGSGSGSPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSA

SEQ ID NO: 24 - protein, 301 aa, synthetic construct
MHPEPAPPPSRSSPELPPSGGSTTSGSRRSRRRSGDGEPPGAPPPPPSAVTYPDWIGQSYSEVMSLNEHSMQALSWRKLYLSRAKLKASSRTSALLSGFAMVAMVEVQLDADHDYPPGLLIAFSACTTVLVAVHLFALMISTCILPNIEAVSNVHNLNSVKESPHERMHRHIELAWAFSTVIGTLLFLAEVVLLCWVKFLPLKKQPGQPRPTSKPPASGAAANVSTSGITPGQAAAIASTTIMVPFGLIFIVFAVHFYRSLVSHKTDRQFQELNELAEFARLQDQLDHRGDHPLTPGSHYA

SEQ ID NO: 25 - protein, 728 aa, synthetic construct
MRSAAVLALLLCAGQVTALPVNSPMNKGDTEVMKCIVEVISDTLSKPSPMPVSQECFETLRGDERILSILRHQNLLKELQDLALQGAKERAHQQKKHSGFEDELSEVLENQSSQAELKEAVEEPSSKDVMEKREDSKEAEKSGEATDGARPQALPEPMQESKAEGNNQAPGEEEEEEEEATNTHPPASLPSQKYPGPQAEGDSEGLSQGLVDREKGLSAEPGWQAKREEEEEEEEEAEAGEEAVPEEEGPTVVLNPHPSLGYKEIRKGESRSEALAVDGAGKPGAEEAQDPEGKGEQEHSQQKEEEEEMAVVPQGLFRGGKSGELEQEEERLSKEWEDSKRWSKMDQLAKELTAEKRLEGQEEEEDNRDSSMKLSFRARAYGFRGPGPQLRRGWRPSSREDSLEAGLPLQVRGYPEEKKEEEGSANRRPEDQELESLSAIEAELEKVAHQLQALRRGGSGESNQSNNGGSGNAALNRGGRYVPPHLRGGSGSMVSKGEENNLAIIKEFMRFKVHMEGSVNGHEFEIEGEGEGRPYEGTQTAKLKVTKGGPLPFAWDILSPQFMYGSKAYVKHPADIPDYLKLSFPEGFNWERVMNFEDGGVVTVTQDSSLQDGEFIYKVKLRGTNFPSDGPVMQCRTMGWEASTERMYPEDGALKGEIKQRLKLKDGGHYDAEVKTTYKAKKPVQLPGAYNVDIKLDILSHNEDYTIVEQYERAEGRHSTGGMDELYK

SEQ ID NO: 26 - protein, 277 aa, synthetic construct
MVELKRSEEQKLISEEDLLRSEEQKLISEEDLLRSEEQKLISEEDLLGNSSSWYAPEALQKWLQLTHEVEVQYYNIKKQNAEKQLLVAKEGAEKIKKKRNTLFGTFHVAHSSSLDDVDHKILTAKQALSEVTAALRERLHRWQQIEILCGFQIVNNPGIHGTGSGSGSGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 27 - protein, 263 aa, synthetic construct
MVELKRSEEQKLISEEDLLRSEEQKLISEEDLLRSEEQKLISEEDLLGNSSSWYAPEALQKWLQLTHEVEVQYYNIKKQNAEKQLLVAKEGAEKIKKKRNTLFGTFHVAHSSSLDDVDHKILTAKQALSEVTAALRERLHRWQQIEILCGFQIVNNPGIHGTGSGSGSGSILWHEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 28 - protein, 458 aa, synthetic construct
MGNCSYPYDVPDYAGSYPYDVPDYAGSYPYDVPDYAGENSMHPEPAPPPSRSSPELPPSGGSTTSGSRRSRRRSGDGEPPGAPPPPPSAVTYPDWIGQSYSEVMSLNEHSMQALSWRKLYLSRAKLKASSRTSALLSGFAMVAMVEVQLDADHDYPPGLLIAFSACTTVLVAVHLFALMISTCILPNIEAVSNVHNLNSVKESPHERMHRHIELAWAFSTVIGTLLFLAEVVLLCWVKFLPLKKQPGQPRPTSKPPASGAAANVSTSGITPGQAAAIASTTIMVPFGLIFIVFAVHFYRSLVSHKTDRQFQELNELAEFARLQDQLDHRGDHPLTPGSHYAGTGSGSGSGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 29 - protein, 110 aa, synthetic construct
SSWYAPEALQKWLQLTHEVEVQYYNIKKQNVEKQLLVAKEGAEKIKKKRNTLFGTFHVAHSSSLDDVDHKILTAKQALSEVTAALRERLHRWQQIEILCGFQIVNNPGIH

SEQ ID NO: 30 - protein, 110 aa, synthetic construct
SSWYAPEALQKWLQLTHEVEVQYYNIKKQNDEKQLLVAKEGAEKIKKKRNTLFGTFHVAHSSSLDDVDHKILTAKQALSEVTAALRERLHRWQQIEILCGFQIVNNPGIH

SEQ ID NO: 31 - protein, 110 aa, synthetic construct
SSWYAPEALQKWLQLTHEVEVQYYNIKKQNKEKQLLVAKEGAEKIKKKRNTLFGTFHVAHSSSLDDVDHKILTAKQALSEVTAALRERLHRWQQIEILCGFQIVNNPGIH

SEQ ID NO: 32 - protein, 110 aa, synthetic construct
SSWYAPEALQKWLQLTHEVEVQYYNIKKQNSEKQLLVAKEGAEKIKKKRNTLFGTFHVAHSSSLDDVDHKILTAKQALSEVTAALRERLHRWQQIEILCGFQIVNNPGIH

SEQ ID NO: 33 - protein, 110 aa, synthetic construct
SSWYAPEALQKWLQLTHEVEVQYYNIKKQNGEKQLLVAKEGAEKIKKKRNTLFGTFHVAHSSSLDDVDHKILTAKQALSEVTAALRERLHRWQQIEILCGFQIVNNPGIH

SEQ ID NO: 34 - protein, 110 aa, synthetic construct
SSWYAPEALQKWLQLTHEVEVQYYNIKKQNAEKQALVAKEGAEKIKKKRNTLFGTFHVAHSSSLDDVDHKILTAKQALSEVTAALRERLHRWQQIEILCGFQIVNNPGIH

SEQ ID NO: 35 - protein, 110 aa, synthetic construct
SSWYAPEALQKWLQLTHEVEVQYYNIKKQNAEKQVLVAKEGAEKIKKKRNTLFGTFHVAHSSSLDDVDHKILTAKQALSEVTAALRERLHRWQQIEILCGFQIVNNPGIH

SEQ ID NO: 36 - protein, 110 aa, synthetic construct
SSWYAPEALQKWLQLTHEVEVQYYNIKKQNAEKQSLVAKEGAEKIKKKRNTLFGTFHVAHSSSLDDVDHKILTAKQALSEVTAALRERLHRWQQIEILCGFQIVNNPGIH

SEQ ID NO: 37 - protein, 110 aa, synthetic construct
SSWYAPEALQKWLQLTHEVEVQYYNIKKQNAEKQLLVDKEGAEKIKKKRNTLFGTFHVAHSSSLDDVDHKILTAKQALSEVTAALRERLHRWQQIEILCGFQIVNNPGIH

SEQ ID NO: 38 - protein, 110 aa, synthetic construct
SSWYAPEALQKWLQLTHEVEVQYYNIKKQNAEKQLLVKKEGAEKIKKKRNTLFGTFHVAHSSSLDDVDHKILTAKQALSEVTAALRERLHRWQQIEILCGFQIVNNPGIH

SEQ ID NO: 39 - protein, 110 aa, synthetic construct
SSWYAPEALQKWLQLTHEVEVQYYNIKKQNAEKQLLVSKEGAEKIKKKRNTLFGTFHVAHSSSLDDVDHKILTAKQALSEVTAALRERLHRWQQIEILCGFQIVNNPGIH

SEQ ID NO: 40 - protein, 110 aa, synthetic construct
SSWYAPEALQKWLQLTHEVEVQYYNIKKQNAEKQLLVGKEGAEKIKKKRNTLFGTFHVAHSSSLDDVDHKILTAKQALSEVTAALRERLHRWQQIEILCGFQIVNNPGIH

SEQ ID NO: 41 - protein, 110 aa, synthetic construct
SSWYAPEALQKWLQLTHEVEVQYYNIKKQNAEKQLLVAKEGDEKIKKKRNTLFGTFHVAHSSSLDDVDHKILTAKQALSEVTAALRERLHRWQQIEILCGFQIVNNPGIH

SEQ ID NO: 42 - protein, 110 aa, synthetic construct
SSWYAPEALQKWLQLTHEVEVQYYNIKKQNAEKQLLVAKEGKEKIKKKRNTLFGTFHVAHSSSLDDVDHKILTAKQALSEVTAALRERLHRWQQIEILCGFQIVNNPGIH

SEQ ID NO: 43 - protein, 110 aa, synthetic construct
SSWYAPEALQKWLQLTHEVEVQYYNIKKQNAEKQLLVAKEGSEKIKKKRNTLFGTFHVAHSSSLDDVDHKILTAKQALSEVTAALRERLHRWQQIEILCGFQIVNNPGIH

SEQ ID NO: 44 - protein, 110 aa, synthetic construct
SSWYAPEALQKWLQLTHEVEVQYYNIKKQNAEKQLLVAKEGGEKIKKKRNTLFGTFHVAHSSSLDDVDHKILTAKQALSEVTAALRERLHRWQQIEILCGFQIVNNPGIH

SEQ ID NO: 45 - protein, 259 aa, synthetic construct
MWWRLWWLLLLLLLLWPMVWAGSMVSKGEENNLAIIKEFMRFKVHMEGSVNGHEFEIEGEGEGRPYEGTQTAKLKVTKGGPLPFAWDILSPQFMYGSKAYVKHPADIPDYLKLSFPEGFNWERVMNFEDGGVVTVTQDSSLQDGEFIYKVKLRGTNFPSDGPVMQCRTMGWEASTERMYPEDGALKGEIKQRLKLKDGGHYDAEVKTTYKAKKPVQLPGAYNVDIKLDILSHNEDYTIVEQYERAEGRHSTGGMDELYK

SEQ ID NO: 46 - protein, 433 aa, synthetic construct
MWWRLWWLLLLLLLLWPMVWAGSAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDRGSGSGSGSMVSKGEENNLAIIKEFMRFKVHMEGSVNGHEFEIEGEGEGRPYEGTQTAKLKVTKGGPLPFAWDILSPQFMYGSKAYVKHPADIPDYLKLSFPEGFNWERVMNFEDGGVVTVTQDSSLQDGEFIYKVKLRGTNFPSDGPVMQCRTMGWEASTERMYPEDGALKGEIKQRLKLKDGGHYDAEVKTTYKAKKPVQLPGAYNVDIKLDILSHNEDYTIVEQYERAEGRHSTGGMDELYK

SEQ ID NO: 47 - protein, 682 aa, synthetic construct
MWWRLWWLLLLLLLLWPMVWAGSYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLTGSGSGSGSMVSKGEENNLAIIKEFMRFKVHMEGSVNGHEFEIEGEGEGRPYEGTQTAKLKVTKGGPLPFAWDILSPQFMYGSKAYVKHPADIPDYLKLSFPEGFNWERVMNFEDGGVVTVTQDSSLQDGEFIYKVKLRGTNFPSDGPVMQCRTMGWEASTERMYPEDGALKGEIKQRLKLKDGGHYDAEVKTTYKAKKPVQLPGAYNVDIKLDILSHNEDYTIVEQYERAEGRHSTGGMDELYK

SEQ ID NO: 48 - protein, 701 aa, synthetic construct
MRSAAVLALLLCAGQVTALPVNSPMNKGDTEVMKCIVEVISDTLSKPSPMPVSQECFETLRGDERILSILRHQNLLKELQDLALQGAKERAHQQKKHSGFEDELSEVLENQSSQAELKEAVEEPSSKDVMEKREDSKEAEKSGEATDGARPQALPEPMQESKAEGNNQAPGEEEEEEEEATNTHPPASLPSQKYPGPQAEGDSEGLSQGLVDREKGLSAEPGWQAKREEEEEEEEEAEAGEEAVPEEEGPTVVLNPHPSLGYKEIRKGESRSEALAVDGAGKPGAEEAQDPEGKGEQEHSQQKEEEEEMAVVPQGLFRGGKSGELEQEEERLSKEWEDSKRWSKMDQLAKELTAEKRLEGQEEEEDNRDSSMKLSFRARAYGFRGPGPQLRRGWRPSSREDSLEAGLPLQVRGYPEEKKEEEGSANRRPEDQELESLSAIEAELEKVAHQLQALRRGGSGSGSGSMVSKGEENNLAIIKEFMRFKVHMEGSVNGHEFEIEGEGEGRPYEGTQTAKLKVTKGGPLPFAWDILSPQFMYGSKAYVKHPADIPDYLKLSFPEGFNWERVMNFEDGGVVTVTQDSSLQDGEFIYKVKLRGTNFPSDGPVMQCRTMGWEASTERMYPEDGALKGEIKQRLKLKDGGHYDAEVKTTYKAKKPVQLPGAYNVDIKLDILSHNEDYTIVEQYERAEGRHSTGGMDELYK

SEQ ID NO: 49 - protein, 721 aa, synthetic construct
MRSAAVLALLLCAGQVTALPVNSPMNKGDTEVMKCIVEVISDTLSKPSPMPVSQECFETLRGDERILSILRHQNLLKELQDLALQGAKERAHQQKKHSGFEDELSEVLENQSSQAELKEAVEEPSSKDVMEKREDSKEAEKSGEATDGARPQALPEPMQESKAEGNNQAPGEEEEEEEEATNTHPPASLPSQKYPGPQAEGDSEGLSQGLVDREKGLSAEPGWQAKREEEEEEEEEAEAGEEAVPEEEGPTVVLNPHPSLGYKEIRKGESRSEALAVDGAGKPGAEEAQDPEGKGEQEHSQQKEEEEEMAVVPQGLFRGGKSGELEQEEERLSKEWEDSKRWSKMDQLAKELTAEKRLEGQEEEEDNRDSSMKLSFRARAYGFRGPGPQLRRGWRPSSREDSLEAGLPLQVRGYPEEKKEEEGSANRRPEDQELESLSAIEAELEKVAHQLQALRRGGSGSAGGDDRRGGAGGGGYRRGGGNSGSMVSKGEENNLAIIKEFMRFKVHMEGSVNGHEFEIEGEGEGRPYEGTQTAKLKVTKGGPLPFAWDILSPQFMYGSKAYVKHPADIPDYLKLSFPEGFNWERVMNFEDGGVVTVTQDSSLQDGEFIYKVKLRGTNFPSDGPVMQCRTMGWEASTERMYPEDGALKGEIKQRLKLKDGGHYDAEVKTTYKAKKPVQLPGAYNVDIKLDILSHNEDYTIVEQYERAEGRHSTGGMDELYK

SEQ ID NO: 50 - protein, 723 aa, synthetic construct
MRSAAVLALLLCAGQVTALPVNSPMNKGDTEVMKCIVEVISDTLSKPSPMPVSQECFETLRGDERILSILRHQNLLKELQDLALQGAKERAHQQKKHSGFEDELSEVLENQSSQAELKEAVEEPSSKDVMEKREDSKEAEKSGEATDGARPQALPEPMQESKAEGNNQAPGEEEEEEEEATNTHPPASLPSQKYPGPQAEGDSEGLSQGLVDREKGLSAEPGWQAKREEEEEEEEEAEAGEEAVPEEEGPTVVLNPHPSLGYKEIRKGESRSEALAVDGAGKPGAEEAQDPEGKGEQEHSQQKEEEEEMAVVPQGLFRGGKSGELEQEEERLSKEWEDSKRWSKMDQLAKELTAEKRLEGQEEEEDNRDSSMKLSFRARAYGFRGPGPQLRRGWRPSSREDSLEAGLPLQVRGYPEEKKEEEGSANRRPEDQELESLSAIEAELEKVAHQLQALRRGGSGYNGGGGGGGNRGYNNNRGGGGGGYNGSMVSKGEENNLAIIKEFMRFKVHMEGSVNGHEFEIEGEGEGRPYEGTQTAKLKVTKGGPLPFAWDILSPQFMYGSKAYVKHPADIPDYLKLSFPEGFNWERVMNFEDGGVVTVTQDSSLQDGEFIYKVKLRGTNFPSDGPVMQCRTMGWEASTERMYPEDGALKGEIKQRLKLKDGGHYDAEVKTTYKAKKPVQLPGAYNVDIKLDILSHNEDYTIVEQYERAEGRHSTGGMDELYK

SEQ ID NO: 51 - protein, 751 aa, synthetic construct
MRSAAVLALLLCAGQVTALPVNSPMNKGDTEVMKCIVEVISDTLSKPSPMPVSQECFETLRGDERILSILRHQNLLKELQDLALQGAKERAHQQKKHSGFEDELSEVLENQSSQAELKEAVEEPSSKDVMEKREDSKEAEKSGEATDGARPQALPEPMQESKAEGNNQAPGEEEEEEEEATNTHPPASLPSQKYPGPQAEGDSEGLSQGLVDREKGLSAEPGWQAKREEEEEEEEEAEAGEEAVPEEEGPTVVLNPHPSLGYKEIRKGESRSEALAVDGAGKPGAEEAQDPEGKGEQEHSQQKEEEEEMAVVPQGLFRGGKSGELEQEEERLSKEWEDSKRWSKMDQLAKELTAEKRLEGQEEEEDNRDSSMKLSFRARAYGFRGPGPQLRRGWRPSSREDSLEAGLPLQVRGYPEEKKEEEGSANRRPEDQELESLSAIEAELEKVAHQLQALRRGGSGESNQSNNGGSGNAALNRGGRYVPPHLRGGSSAGGDDRRGGAGGGGYRRGGGNSGSMVSKGEENNLAIIKEFMRFKVHMEGSVNGHEFEIEGEGEGRPYEGTQTAKLKVTKGGPLPFAWDILSPQFMYGSKAYVKHPADIPDYLKLSFPEGFNWERVMNFEDGGVVTVTQDSSLQDGEFIYKVKLRGTNFPSDGPVMQCRTMGWEASTERMYPEDGALKGEIKQRLKLKDGGHYDAEVKTTYKAKKPVQLPGAYNVDIKLDILSHNEDYTIVEQYERAEGRHSTGGMDELYK

SEQ ID NO: 52 - protein, 776 aa, synthetic construct
MRSAAVLALLLCAGQVTALPVNSPMNKGDTEVMKCIVEVISDTLSKPSPMPVSQECFETLRGDERILSILRHQNLLKELQDLALQGAKERAHQQKKHSGFEDELSEVLENQSSQAELKEAVEEPSSKDVMEKREDSKEAEKSGEATDGARPQALPEPMQESKAEGNNQAPGEEEEEEEEATNTHPPASLPSQKYPGPQAEGDSEGLSQGLVDREKGLSAEPGWQAKREEEEEEEEEAEAGEEAVPEEEGPTVVLNPHPSLGYKEIRKGESRSEALAVDGAGKPGAEEAQDPEGKGEQEHSQQKEEEEEMAVVPQGLFRGGKSGELEQEEERLSKEWEDSKRWSKMDQLAKELTAEKRLEGQEEEEDNRDSSMKLSFRARAYGFRGPGPQLRRGWRPSSREDSLEAGLPLQVRGYPEEKKEEEGSANRRPEDQELESLSAIEAELEKVAHQLQALRRGGSGESNQSNNGGSGNAALNRGGRYVPPHLRGGSSAGGDDRRGGAGGGGYRRGGGNSYNGGGGGGGNRGYNNNRGGGGGGYNGSMVSKGEENNLAIIKEFMRFKVHMEGSVNGHEFEIEGEGEGRPYEGTQTAKLKVTKGGPLPFAWDILSPQFMYGSKAYVKHPADIPDYLKLSFPEGFNWERVMNFEDGGVVTVTQDSSLQDGEFIYKVKLRGTNFPSDGPVMQCRTMGWEASTERMYPEDGALKGEIKQRLKLKDGGHYDAEVKTTYKAKKPVQLPGAYNVDIKLDILSHNEDYTIVEQYERAEGRHSTGGMDELYK

SEQ ID NO: 53 - protein, 236 aa, synthetic construct
MVSKGEENNLAIIKEFMRFKVHMEGSVNGHEFEIEGEGEGRPYEGTQTAKLKVTKGGPLPFAWDILSPQFMYGSKAYVKHPADIPDYLKLSFPEGFNWERVMNFEDGGVVTVTQDSSLQDGEFIYKVKLRGTNFPSDGPVMQCRTMGWEASTERMYPEDGALKGEIKQRLKLKDGGHYDAEVKTTYKAKKPVQLPGAYNVDIKLDILSHNEDYTIVEQYERAEGRHSTGGMDELYK

SEQ ID NO: 54 - protein, 10 aa, synthetic construct
MVSKGEENNL

SEQ ID NO: 55 - protein, 10 aa, synthetic construct
STGGMDELYK

SEQ ID NO: 56 - protein, 166 aa, synthetic construct
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 57 - protein, 415 aa, synthetic construct
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 58 - protein, 457 aa, synthetic construct
MRSAAVLALLLCAGQVTALPVNSPMNKGDTEVMKCIVEVISDTLSKPSPMPVSQECFETLRGDERILSILRHQNLLKELQDLALQGAKERAHQQKKHSGFEDELSEVLENQSSQAELKEAVEEPSSKDVMEKREDSKEAEKSGEATDGARPQALPEPMQESKAEGNNQAPGEEEEEEEEATNTHPPASLPSQKYPGPQAEGDSEGLSQGLVDREKGLSAEPGWQAKREEEEEEEEEAEAGEEAVPEEEGPTVVLNPHPSLGYKEIRKGESRSEALAVDGAGKPGAEEAQDPEGKGEQEHSQQKEEEEEMAVVPQGLFRGGKSGELEQEEERLSKEWEDSKRWSKMDQLAKELTAEKRLEGQEEEEDNRDSSMKLSFRARAYGFRGPGPQLRRGWRPSSREDSLEAGLPLQVRGYPEEKKEEEGSANRRPEDQELESLSAIEAELEKVAHQLQALRRG

SEQ ID NO: 59 - protein, 10 aa, synthetic construct
MRSAAVLALL

SEQ ID NO: 60 - protein, 10 aa, synthetic construct
AHQLQALRRG

SEQ ID NO: 61 - protein, 30 aa, synthetic construct
ESNQSNNGGSGNAALNRGGRYVPPHLRGGS

SEQ ID NO: 62 - protein, 23 aa, synthetic construct
SAGGDDRRGGAGGGGYRRGGGNS

SEQ ID NO: 63 - protein, 25 aa, synthetic construct
YNGGGGGGGNRGYNNNRGGGGGGYN

SEQ ID NO: 64 - protein, 53 aa, synthetic construct
ESNQSNNGGSGNAALNRGGRYVPPHLRGGSSAGGDDRRGGAGGGGYRRGGGNS

SEQ ID NO: 65 - protein, 78 aa, synthetic construct
ESNQSNNGGSGNAALNRGGRYVPPHLRGGSSAGGDDRRGGAGGGGYRRGGGNSYNGGGGGGGNRGYNNNRGGGGGGYN

SEQ ID NO: 66 - protein, 110 aa, synthetic construct
SSWYAPEALQKWLQLTHEVEVQYYNIKKQNAEKQLLVAKEGAEKIKKKRNTLFGTFHVAHSSSLDDVDHKILTAKQALSEVTAALRERLHRWQQIEILCGFQIVNNPGIH

SEQ ID NO: 67 - protein, 50 aa, synthetic construct
MVELKRSEEQKLISEEDLLRSEEQKLISEEDLLRSEEQKLISEEDLLGNS

SEQ ID NO: 68 - protein, 10 aa, synthetic construct
SSWYAPEALQ

SEQ ID NO: 69 - protein, 10 aa, synthetic construct
FQIVNNPGIH

SEQ ID NO: 70 - protein, 10 aa, synthetic construct
GTGSGSGSGS

SEQ ID NO: 71 - protein, 93 aa, synthetic construct
ILWHEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 72 - protein, 301 aa, synthetic construct; identical to SEQ ID NO: 24

SEQ ID NO: 73 - protein, 10 aa, synthetic construct
MHPEPAPPPS

SEQ ID NO: 74 - protein, 10 aa, synthetic construct
HPLTPGSHYA

SEQ ID NO: 75 - protein, 40 aa, synthetic construct
MGNCSYPYDVPDYAGSYPYDVPDYAGSYPYDVPDYAGENS

SEQ ID NO: 76 - protein, 10 aa, synthetic construct
GVQVETISPG

SEQ ID NO: 77 - protein, 10 aa, synthetic construct
VFDVELLKLE

SEQ ID NO: 78 - protein, 51 aa, synthetic construct
MAVELKRSEEQKLISEEDLLRSEEQKLISEEDLLRSEEQKLISEEDLLGNS

SEQ ID NO: 79 - protein, 59 aa, synthetic construct
GSGSGSGSGSGSSGSGSGGSSGSGGSSSGGSGSGSGSSGGSGGGGSGGSSGSSSGGGGS

SEQ ID NO: 80 - protein, 258 aa, synthetic construct
PDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSA

SEQ ID NO: 81 - protein, 20 aa, synthetic construct
GSGSGSGSGSGSGSGSGSGS

SEQ ID NO: 82 - protein, 5 aa, synthetic construct
PDPAA

SEQ ID NO: 83 - protein, 5 aa, synthetic construct
PNSSA

SEQ ID NO: 84 - protein, 23 aa, synthetic construct
MWWRLWWLLLLLLLLWPMVWAGS

SEQ ID NO: 85 - protein, 166 aa, synthetic construct; identical to SEQ ID NO: 56

SEQ ID NO: 86 - protein, 415 aa, synthetic construct; identical to SEQ ID NO: 57

SEQ ID NO: 87 - protein, 457 aa, synthetic construct; identical to SEQ ID NO: 58

SEQ ID NO: 88 - protein, 35 aa, synthetic construct
GSGESNQSNNGGSGNAALNRGGRYVPPHLRGGSGS

SEQ ID NO: 89 - protein, 28 aa, synthetic construct
GSGSAGGDDRRGGAGGGGYRRGGGNSGS

SEQ ID NO: 90 - protein, 30 aa, synthetic construct
GSGYNGGGGGGGNRGYNNNRGGGGGGYNGS

SEQ ID NO: 91 - protein, 58 aa, synthetic construct
GSGESNQSNNGGSGNAALNRGGRYVPPHLRGGSSAGGDDRRGGAGGGGYRRGGGNSGS

SEQ ID NO: 92 - protein, 83 aa, synthetic construct
GSGESNQSNNGGSGNAALNRGGRYVPPHLRGGSSAGGDDRRGGAGGGGYRRGGGNSYNGGGGGGGNRGYNNNRGGGGGGYNGS

SEQ ID NO: 93 - protein, 107 aa, synthetic construct
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE